(19) United States
(12) Patent Application Publication
(10) Pub. No.: US 2017/0210738 A1
(43) Pub. Date: Jul. 27, 2017

(54) GLUCOSE TRANSPORT INHIBITORS

(71) Applicant: Bayer Pharma Aktiengesellschaft, Berlin (DE)

(72) Inventors: Bernd BUCHMANN, Hohen Neuendorf (DE); Iring HEISLER, Düsseldorf (DE); Thomas MÜLLER, Langenfeld (DE); Arwed CLEVE, Berlin (DE); Melanie HEROULT, Düsseldorf (DE); Roland NEUHAUS, Berlin (DE); Heike PETRUL, Berlin (DE); Maria QUANZ-SCHÖFFEL, Berlin (DE)

(73) Assignee: Bayer Pharma Aktiengesellschaft, Berlin (DE)

(21) Appl. No.: 15/328,455

(22) PCT Filed: Jul. 22, 2015
(86) PCT No.: PCT/EP2015/066706

§ 371 Date: Jan. 23, 2017

(30) Foreign Application Priority Data
Jul. 24, 2014 (EP) 14178311.8

(51) Int. Cl. C07D 417/14; C07D 413/14; C07D 401/14; C07D 401/12; A61K 31/4709

(52) U.S. Cl. CPC C07D 417/14; C07D 401/12; A61K 31/4709; C07D 401/14; C07D 413/14

(57) ABSTRACT

The present invention relates to chemical compounds of formula (I) that selectively inhibit glucose transporter 1 (GLUT1), to methods of preparing said compounds, to pharmaceutical compositions and combinations comprising said compounds, to the use of said compounds for manufacturing a pharmaceutical composition for the treatment or prophylaxis of a disease, as well as to intermediate compounds useful in the preparation of said compounds.

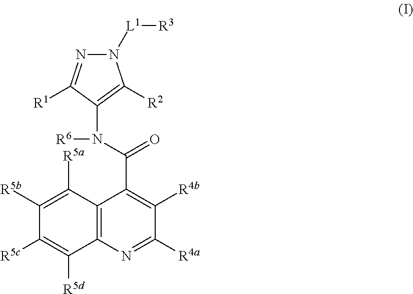

The present invention relates to chemical compounds that selectively inhibit glucose transporter 1 (GLUT1), to methods of preparing said compounds, to pharmaceutical compositions and combinations comprising said compounds, to the use of said compounds for manufacturing a pharmaceutical composition for the treatment or prophylaxis of a disease, as well as to intermediate compounds useful in the preparation of said compounds.

### BACKGROUND OF THE INVENTION

Glucose is an essential substrate for metabolism in most cells. Because glucose is a polar molecule, transport through biological membranes requires specific transport proteins. Transport of glucose through the apical membrane of intestinal and kidney epithelial cells depends on the presence of secondary active Na⁺/glucose symporters, SGLT-1 and SGLT-2, which concentrate glucose inside the cells, using the energy provided by co-transport of Na⁺ ions down their electrochemical gradient. Facilitated diffusion of glucose through the cellular membrane is otherwise catalyzed by glucose carriers (protein symbol GLUT, gene symbol SLC2 for Solute Carrier Family 2) that belong to a superfamily of transport facilitators (major facilitator superfamily) including organic anion and cation transporters, yeast hexose transporter, plant hexose/proton symporters, and bacterial sugar/proton symporters.

Basal glucose transporters (GLUTs) function as glucose channels and are required for maintaining the basic glucose needs of cells. These GLUTs are constitutively expressed and functional in cells and are not regulated by (or sensitive to) insulin. ALL cells use both glycolysis and oxidative phosphorylation in mitochondria but rely overwhelmingly on oxidative phosphorylation when oxygen is abundant, switching to glycolysis at times of oxygen deprivation (hypoxia), as it occurs in cancer. In glycolysis, glucose is converted to pyruvate and two ATP molecules are generated in the process. Cancer cells, because of their faster proliferation rates, are predominantly in a hypoxic (low oxygen) state. Therefore, cancer cells use glycolysis (lactate formation) as their predominant glucose metabolism pathway. Such a glycolytic switch not only gives cancer higher potentials for metastasis and invasiveness, but also increases cancer's vulnerability to external interference in glycolysis. The reduction of basal glucose transport is likely to restrict glucose supply to cancer cells, leading to glucose deprivation that forces cancer cells to slow down growth or to starve.

All known GLUT proteins contain 12 transmembrane domains and transport glucose by facilitating diffusion, an energy-independent process. GLUT1 transports glucose into cells probably by alternating its conformation. According to this model, GLUT1 exposes a single substrate-binding site toward either the outside or the inside of the cell. Binding of glucose to one site triggers a conformational change, releasing glucose to the other side of the membrane. Results of transgenic and knockout animal studies support an important role for these transporters in the control of glucose utilization, glucose storage and glucose sensing. The GLUT proteins differ in their kinetics and are tailored to the needs of the cell types they serve. Although more than one GLUT protein may be expressed by a particular cell type, cancers frequently overexpress GLUT1, which is a high affinity glucose transporter, and its expression level is correlated with invasiveness and metastasis potentials of cancers, indicating the importance of upregulation of glucose transport in cancer cell growth and in the severity of cancer malignancy. GLUT1 expression was also found to be significantly higher than that of any other glucose transporters.

Evidence indicates that cancer cells are more sensitive to glucose deprivation than normal cells. Numerous studies strongly suggest that basal glucose transport inhibition induces apoptosis and blocks cancer cell growth. Anti-angiogenesis has been shown to be a very effective way to restrict cancer growth and cause cancer ablation.

Reduced GLUT1 expression following transfection of GLUT1 antisense cDNA into cancer cell lines has been shown to suppress cell growth in vitro and tumor growth in vivo, and to reduce in vitro invasiveness of cells (Noguchi Y. et al. Cancer Lett 154(2), 2000, 175-182; Ito S. et al. J Natl Cancer Inst 94(14), 2002, 1080-1091).

It has been demonstrated that GLUT1 is the most highly expressed hexose transporter in ErbB2- and PyVMT-induced mouse mammary carcinoma models, and that reducing the level of GLUT1 using shRNA or Cre/Lox results in reduced glucose usage, reduced growth on plastic and in soft agar, and impaired tumor growth in nude mice (Christian D. Young et al., PLoS ONE, August 2011, Volume 6, Issue 8, e23205, 1-12).

Therefore, inhibition of GLUT1 represents a promising approach for the treatment of proliferative disorders including solid tumours such as carcinomas and sarcomas and leukaemias and lymphoid malignancies or other disorders associated with uncontrolled cellular proliferation.

Different compounds have been disclosed in prior art which show an inhibitory effect on GLUT1. For example, WO2011/119866(A1) discloses composition and methods for glucose transport inhibition; WO2012/051117(A2) and WO2013/155338(A2) disclose substituted benzamides as GLUT1 inhibitors.

Compounds showing a certain structural similarity to the compounds of the present invention are disclosed in prior art. WO97/36881(A1) discloses arylheteroaryl-containing compounds which inhibit farnesyl-protein transferase. WO00/07996(A2) discloses pyrazole estrogen receptor agonist and antagonist compounds. WO01/21160(A2) discloses carboxamide derivatives as inhibitors of herpesviridae. WO03/037274(A2) and WO2004/099154(A2) disclose pyrazole-amides as inhibitors of sodium channels. WO2004/098528(A2) discloses pyrazole derived compounds as inhibitors of p38 kinase. WO2006/132197(A1) discloses heterocyclic compounds as inhibitors of 11 β-hydroxysteroid dehydrogenase type 1. WO2006/062249(A1) discloses compounds for the prevention, therapy or improvement of a disease to which the activation of a thrombopoietin receptor is effective. WO2008/126899(A1) discloses 5-membered heterocyclic compounds as inhibitors of xanthine oxidase. WO2008/008286(A2) discloses substituted pyrazoles as ghrelin receptor antagonists. WO2009/025793(A2) discloses compounds that function as bitter taste blockers. WO2009/027393(A2) and WO2010/034737(A1) disclose pyrazole compounds for controlling invertebrate pests. WO2009/099193(A1) discloses compounds having inhibitory action on melanin production. WO2009/119880(A1) discloses pyrazole derivatives having an androgen receptor antagonistic action. WO2011/050305(A1) and WO2011/050316(A1) disclose pyrazole compounds as allosteric modulators of mGluR4 receptor activity. WO2011/126903(A2) discloses multisubstituted aromatic compounds including substituted pyrazolyl as thrombin inhibitors. WO2004/110350(A2) discloses compounds modulating amyloid beta. WO2009/055917(A1) discloses inhibitors of histone deacetylase. WO02/23986(A1) discloses 4-acylaminopyrazole derivatives exhibiting fungicidal activities. WO03/051833(A2) discloses heteroaryl substituted pyrazole compounds as mGluR5 modulators. WO2009/076454(A2) discloses compounds which modulate the activity of store-operated calcium channels. WO99/32454(A1) discloses nitrogen containing heteroaromatics with ortho-substituted P1 groups as factor Xa inhibitors. WO2004/037248(A2) and WO2004/043951(A1) discloses compounds as modulators of the peroxisome proliferator activated receptors. WO 2014031936 discloses heterocyclic compounds as modulators of HIF pathway activity.

However, the state of the art described above does not specifically disclose the compounds of general formula (I) of the present invention, or a tautomer, a stereoisomer, an N-oxide, a hydrate, a solvate, or a salt thereof, or a mixture of same, as described and defined herein, and as hereinafter referred to as “compounds of the present invention”, or their pharmacological activity.

### SUMMARY OF THE INVENTION

The present invention covers compounds of general formula (I):

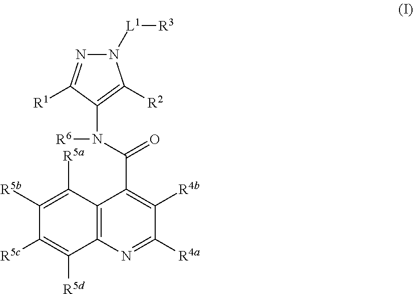

in which:
R¹ represents a hydrogen atom;
R² represents a C₁-C₃-alkyl-, halo-C₁-C₃-alkyl-, cyano-, —C(═O)O—R¹⁰ or —C(═O)N(R^{10a})R^{10b} group;
R³ represents a group selected from: phenyl-, heteroaryl-, C₅-C₆-cycloalkyl- and 5- to 6-membered heterocycloalkyl-;
   wherein said 5- to 6-membered heterocycloalkyl- group is optionally benzocondensed;
   wherein said phenyl-, heteroaryl-, C₅-C₆-cycloalkyl-, and 5- to 6-membered heterocycloalkyl- group is optionally substituted, one or more times, identically or differently, with -(L²)ₚ-R⁷;
   and wherein two -(L²)ₚ-R⁷ groups, if being present ortho to each other on an aryl- or heteroaryl- group optionally form a bridge selected from: *—C₃-C₅-alkylene-*, *—O(CH₂)₂O—*, *—O(CH₂)O—*, *—O(CF₂)O—*, *—CH₂C(R^{10a})(R^{10b})O—*, *—C(═O)N(R^{10a})CH₂—*, *—N(R^{10a})C(═O)CH₂O—*, *—NHC(═O)NH—*; wherein each * represents the point of attachment to said aryl- or heteroaryl- group;
R^{4a} represents a hydrogen atom or a halogen atom or a group selected from: cyano-, hydroxy-, C₁-C₃-alkyl-, halo-C₁-C₃-alkyl-, C₁-C₃-alkoxy-, C₃-C₇-cycloalkyl-, 4- to 7-membered heterocycloalkyl-, —C(═O)N(R^{10a})R^{10b}, —N(R^{10a})R^{10b};
R^{4b} represents a hydrogen atom or a group selected from: C₁-C₃-alkoxy-, C₁-C₃-alkyl-, cyano-;
or
R^{4a} and together R^{4b} form a —C₃-C₅-alkylene- group;
R^{5a}, R^{5b}, R^{5c}, R^{5d}
   independently from each other represent a hydrogen atom, a halogen atom or a group selected from:
   cyano-, —NO₂, C₁-C₃-alkyl-, halo-C₁-C₃-alkyl-, C₁-C₃-alkoxy-, halo-C₁-C₃-alkoxy-, phenyl-,
   heteroaryl-, —C(═O)R¹⁰, —C(═O)N(H)R¹⁰, —C(═O)N(R^{10a})R^{10b}, —C(═O)O—R¹⁰, —N(R^{10a})R^{10b}, —N(H)C(═O)R¹⁰, —N(R^{10a})C(═O)R^{10b}, —N(H)C(═O)N(R^{10a})R^{10b}, —N(R^{10a})C(═O)N(R^{10b})R^{10c}, —N(R^{10a})C(═O)C(═O)N(R^{10b})R^{10c}, —N(H)C(═O)OR¹⁰, —N(R^{10a})C(═O)OR^{10b}, —N(H)S(═O)₂R¹⁰, —N(R^{10a})S(═O)₂R^{10b}, —OR¹⁰, —O(C═O)R¹⁰, —O(C═O)N(R^{10a})R^{10b}, —O(C═O)OR¹⁰, —SR¹⁰, —S(═O)R¹⁰, —S(═O)₂R¹⁰, —S(═O)₂N(H)R¹⁰, —S(═O)₂N(R^{10a})R^{10b} or —S(═O)(═NR^{10a})R^{10b}, said phenyl- or heteroaryl- group being optionally substituted one or more times, identically or differently, with a group selected from: halo-, cyano-, C₁-C₃-alkyl-, halo-C₁-C₃-alkyl-, C₁-C₃-alkoxy- group;
R⁶ represents a hydrogen atom or group selected from: C₁-C₃-alkyl-, C₁-C₃-alkoxy-(L²)-, hydroxy-C₁-C₃-alkyl-, aryl-(L²)-, heteroaryl-(L²)-;
R⁷ represents a group selected from: oxo, C₁-C₃-alkyl-, C₃-C₇-cycloalkyl-, 4- to 7-membered heterocycloalkyl-, halo-C₁-C₃-alkyl-, C₁-C₃-alkoxy-, halo-C₁-C₃-alkoxy-, —OH, —CN, halo-, —C(═O)R⁸, —C(═O)—O—R⁸, —C(═O)N(R^{8a})R^{8b}, —S(═O)₂R⁸, —S(═O)(═N)R¹¹, phenyl-, 5- to 6-membered heteroaryl-;
R⁸ represents a hydrogen atom or a C₁-C₆-alkyl-, halo-C₁-C₃-alkyl-, cyano-C₁-C₄-alkyl-, C₁-C₃-alkoxy-C₁-C₃-alkyl-, C₃-C₇-cycloalkyl-, phenyl-, 5- to 6-membered heteroaryl- or benzyl- group;
R^{8a}, R^{8b}
   represent, independently from each other, a hydrogen atom, or a C₁-C₁₀-alkyl-, C₃-C₇-cycloalkyl-, (C₃-C₇-cycloalkyl)-(L³)-, C₃-C₆-alkenyl-, C₃-C₆-alkynyl-, 4- to 10-membered heterocycloalkyl-, (4- to 10-membered heterocycloalkyl)-(L³)-, phenyl-, heteroaryl-, phenyl-(L³)-, (phenyl)-O-(L³)-, heteroaryl-(L³)-, or (aryl)-(4- to 10-membered heterocycloalkyl)- group;
   said C₁-C₁₀-alkyl-, C₃-C₇-cycloalkyl-, (C₃-C₇-cycloalkyl)-(L³)-, C₃-C₆-alkenyl-, C₃-C₆-alkynyl-, 4- to 10-membered heterocycloalkyl-, (4- to 10-membered heterocycloalkyl)-(L³)-, phenyl-, heteroaryl-, phenyl-(L³)-, (phenyl)-O-(L³)-, heteroaryl-(L³)-, and (aryl)-(4- to 10-membered heterocycloalkyl)- group being optionally substituted one or more times, identically or differently, with R⁹;
or
R^{8a} and R^{8b}, together with the nitrogen atom they are attached to, represent a 4- to 10-membered heterocycloalkyl-group, said 4- to 10-membered heterocycloalkyl-group being optionally substituted one or more times, identically or differently, with R⁹;
R⁹ represents a halogen atom, or a oxo, C₁-C₃-alkyl-, halo-C₁-C₃-alkyl-, hydroxy-C₁-C₃-alkyl-, —CN, —C(═O)R¹⁰, —C(═O)N(H)R¹⁰, —C(═)N(R^{10a})R^{10b}, —C(═O)O—R¹⁰, —N(R^{10a})R^{10b}, —NO₂, —N(H)C(═O)R¹⁰, —N(R^{10a})C(═O)R^{10b}, —N(H)C(═O)N(R^{10a}) R^{10b}, —N(R^{10a})C(═O)N(R^{10b})R^{10c}, —N(H)C(═O)OR¹⁰, —N(R^{10a})C(═O)OR^{10b}, —N(H)S(═O)₂R¹⁰, —N(R^{10a})S(═O)₂R^{10b}, —OR¹⁰, —O(C═O)R¹⁰, —O(C═O)N(R^{10a})R^{10b}, —O(C═O)OR¹⁰, —SR¹⁰, —S(═O)R¹⁰, —S(═O)₂R¹⁰, —S(═O)₂N(H)R¹⁰, —S(═O)₂N(R^{10a})R^{10b}, —S(═O)(=NR^{10a})R^{10b} or a tetrazolyl- group;
or
   two R⁹ groups present ortho to each other on a phenyl- or heteroaryl-ring form a bridge selected from: *—C₃-C₅-alkylene-*, *—O(CH₂)₂O—*, *—O(CH₂)O—*, *—O(CF₂)O—*, *—CH₂C(R^{10a})(R^{10b})O—*, *—C(═O)N(R^{10a})CH₂—*, *—N(R^{10a})C(═O)CH₂O—*, *—NHC(═O)NH—*; wherein each * represents the point of attachment to said phenyl- or heteroaryl- ring;
R¹⁰, R^{10a}, R^{10b}, R^{10c}
   represent, independently from each other, a hydrogen atom or a group selected from: C₁-C₃-alkyl-, halo-C₁-C₃-alkyl-, hydroxy-C₁-C₃-alkyl-, C₁-C₃-alkoxy-C₁-C₃-alkyl-, C₃-C₇-cycloalkyl-;
R¹¹ represents a hydrogen atom or a cyano-, C₁-C₃-alkyl-, —C(═O)R¹⁰, —C(═O)N(H)R¹⁰, —C(═O)N(R^{10a})R^{10b} or —C(═O)O—R¹⁰ group;
L¹ represents a group selected from: —C₁-C₄-alkylene-, —CH₂—CH═CH—, —C(phenyl)(H)—, —CH₂—CH₂—O—;
L² represents a group selected from: —CH₂—, —CH₂—CH₂—, —CH₂—CH₂—CH₂—;
L³ represents a —C₁-C₆-alkylene- group;
p is an integer of 0 or 1;
   or a tautomer, a stereoisomer, an N-oxide, a hydrate, a solvate, or a salt thereof, or a mixture of same.

The present invention further relates to methods of preparing compounds of general formula (I), to pharmaceutical compositions and combinations comprising said compounds, to the use of said compounds for manufacturing a pharmaceutical composition for the treatment or prophylaxis of a disease, as well as to intermediate compounds useful in the preparation of said compounds.

### DETAILED DESCRIPTION OF THE INVENTION

The terms as mentioned in the present text have preferably the following meanings:

The term “halogen atom” or “halo-” is to be understood as meaning a fluorine, chlorine, bromine or iodine atom.

The term “oxo” is to be understood as preferably meaning an oxygen atom attached to an atom featuring suitable bonding valence, such as a saturated carbon atom or a sulfur atom, by a double bond, resulting in the formation e.g. of a carbonyl group —C(═O)— or a sulfonyl group —S(═O)₂—.

The term “C₁-C₁₀-alkyl-” is to be understood as preferably meaning a linear or branched, saturated, monovalent hydrocarbon group having 1, 2, 3, 4, 5, 6, 7, 8, 9 or 10 carbon atoms, e.g. a methyl-, ethyl-, propyl-, butyl-, pentyl-, hexyl-, iso-propyl-, iso-butyl-, sec-butyl-, tert-butyl-, iso-pentyl-, 2-methylbutyl-, 1-methylbutyl-, 1-ethylpropyl-, 1,2-dimethylpropyl-, neo-pentyl-, 1,1-dimethylpropyl-, 4-methylpentyl-, 3-methylpentyl-, 2-methylpentyl-, 1-methylpentyl-, 2-ethylbutyl-, 1-ethylbutyl-, 3,3-dimethylbutyl-, 2,2-dimethylbutyl-, 1,1-dimethylbutyl-, 2,3-dimethylbutyl-, 1,3-dimethylbutyl-, or 1,2-dimethylbutyl-, heptyl-, octyl-, nonyl- or decyl- group, or an isomer thereof. Particularly, said group has 1, 2, 3, 4, 5 or 6 carbon atoms (“C₁-C₆-alkyl-”), more particularly 1, 2, 3 or 4 carbon atoms (“C₁-C₄-alkyl-”), e.g. a methyl-, ethyl-, propyl-, butyl-, iso-propyl-, iso-butyl-, sec-butyl-, tert-butyl-group, even more particularly 1, 2 or 3 carbon atoms (“C₁-C₃-alkyl-”), e.g. a methyl-, ethyl-, n-propyl- or iso-propyl- group.

The term “—C₁-C₈-alkylene-” is understood as preferably meaning a linear or branched, saturated hydrocarbon chain (or “tether”) having 1, 2, 3, 4, 5, 6, 7 or 8 carbon atoms, e.g. —CH₂— (“methylene” or “—C₁-alkylene-”) or, for example —CH₂—CH₂— (“ethylene” or “—C₂-alkylene-”), —CH₂—CH₂—CH₂—, —C(H)(CH₃)—CH₂— or —C(CH₃)₂—) (“propylene” or “—C₃-alkylene-”), or, for example —CH₂—C(H)(CH₃)—CH₂—, —CH₂—C(CH₃)₂—), —CH₂—CH₂—CH₂—CH₂— (“butylene” or “—C₄-alkylene-”), “—C₅-alkylene-”, e.g. —CH₂—CH₂—CH₂—CH₂—CH₂-(“n-pentylene”), or “—C₆-alkylene-”, e.g. —CH₂—CH₂—CH₂—CH₂—CH₂—CH₂— (“n-hexylene”) group. Particularly, said alkylene tether has 1, 2, 3, 4, or 5 carbon atoms (“—C₁-C₅-alkylene-”), more particularly 1 or 2 carbon atoms (“—C₁-C₂-alkylene-”), or, 3, 4, or 5 carbon atoms(“—C₃-C₅-alkylene-”).

The term “halo-C₁-C₃-alkyl-” is to be understood as preferably meaning a linear or branched, saturated, monovalent hydrocarbon group in which the term “C₁-C₃-alkyl-” is defined supra, and in which one or more of the hydrogen atoms is replaced, in identically or differently, by a halogen atom. Particularly, said halogen atom is F, resulting in a group also referred to as “fluoro-C₁-C₃-alkyl-”. Said halo-C₁-C₃-alkyl- group or fluoro-C₁-C₃-alkyl- group is, for example, —CF₃, —CHF₂, —CH₂F, —CF₂CF₃, or —CH₂CF₃.

The term “cyano-C₁-C₄-alkyl-” is to be understood as preferably meaning a linear or branched, saturated, monovalent hydrocarbon group in which the term “C₁-C₄-alkyl-” is defined supra, and in which one or more of the hydrogen atoms is replaced by a cyano group. Said cyano-C₁-C₄-alkyl- group is, for example, —CH₂CN, —CH₂CH₂—CN, —C(CN)H—CH₃, —C(CN)H—CH₂CN, or —CH₂CH₂CH₂CH₂—CN.

The term “hydroxy-C₁-C₃-alkyl-” is to be understood as preferably meaning a linear or branched, saturated, monovalent hydrocarbon group in which the term “C₁-C₃-alkyl-” is defined supra, and in which one or more of the hydrogen atoms is replaced by a hydroxy group with the proviso that not more than one hydrogen atom attached to a single carbon atom is being replaced. Said hydroxy-C₁-C₃-alkyl- group is, for example, —CH₂OH, —CH₂CH₂—OH, —C(OH)H—CH₃, or —C(OH)H—CH₂OH.

The term “C₁-C₃-alkoxy-” is to be understood as preferably meaning a linear or branched, saturated, monovalent group of formula —O—(C₁-C₃-alkyl-), in which the term “C₁-C₃-alkyl-” is defined supra, e.g. a methoxy-, ethoxy-, n-propoxy-, iso-propoxy-.

The term “halo-C₁-C₃-alkoxy-” is to be understood as preferably meaning a linear or branched, saturated, monovalent C₁-C₃-alkoxy- group, as defined supra, in which one or more of the hydrogen atoms is replaced, in identically or differently, by a halogen atom. Particularly, said halogen atom is F, resulting in a group also referred to as “fluoro-C₁-C₃-alkoxy-”. Said halo-C₁-C₃-alkoxy- group or fluoro-C₁-C₃-alkoxy- group is, for example, —OCF₃, —OCHF₂, —OCH₂F, —OCF₂CF₃, or —OCH₂CF₃.

The term “C₁-C₃-alkoxy-C₁-C₃-alkyl-” is to be understood as preferably meaning a linear or branched, saturated, monovalent C₁-C₃-alkyl- group, as defined supra, in which one or more of the hydrogen atoms is replaced, in identically or differently, by a C₁-C₃-alkoxy group, as defined supra, e.g. methoxyalkyl-, ethoxyalkyl-, propyloxyalkyl- or iso-propoxyalkyl-.

The term “halo-C₁-C₃-alkoxy-C₁-C₃-alkyl-” is to be understood as preferably meaning a linear or branched, saturated, monovalent C₁-C₃-alkoxy-C₁-C₃-alkyl-group, as defined supra, in which one or more of the hydrogen atoms is replaced, in identically or differently, by a halogen atom. Particularly, said halogen atom is F, resulting in a group also referred to as “fluoro-C₁-C₃-alkoxy-C₁-C₃-alkyl-”. Said halo-C₁-C₃-alkoxy-C₁-C₃-alkyl- group or fluoro-C₁-C₃-alkoxy-C₁-C₃-alkyl- group is, for example, —CH₂CH₂OCF₃, —CH₂CH₂OCHF₂, —CH₂CH₂OCH₂F, —CH₂CH₂OCF₂CF₃, or —CH₂CH₂OCH₂CF₃.

The term “C₂-C₆-alkenyl-” is to be understood as preferably meaning a linear or branched, monovalent hydrocarbon group, which contains one or more double bonds, and which has 2, 3, 4, 5 or 6 carbon atoms, particularly 3, 4, 5 or 6 carbon atoms (“C₃-C₆-alkenyl-”), more particularly 3 or 4 carbon atoms (“C₃-C₄-alkenyl-”), it being understood that in the case in which said alkenyl-group contains more than one double bond, then said double bonds may be isolated from, or conjugated with, each other. Said alkenyl- group is, for example, a vinyl-, allyl-, (E)-2-methylvinyl-, (Z)-2-methylvinyl-, homoallyl-, (E)-but-2-enyl-, (Z)-but-2-enyl-, (E)-but-1-enyl-, (Z)-but-1-enyl-, pent-4-enyl-, (E)-pent-3-enyl-, (Z)-pent-3-enyl-, (E)-pent-2-enyl-, (Z)-pent-2-enyl-, (E)-pent-1-enyl-, (Z)-pent-1-enyl-, hex-5-enyl-, (E)-hex-4-enyl-, (Z)-hex-4-enyl-, (E)-hex-3-enyl-, (Z)-hex-3-enyl-, (E)-hex-2-enyl-, (Z)-hex-2-enyl-, (E)-hex-1-enyl-, (Z)-hex-1-enyl-, iso-propenyl-, 2-methylprop-2-enyl-, 1-methylprop-2-enyl-, 2-methylprop-1-enyl-, (E)-1-methylprop-1-enyl-, (Z)-1-methylprop-1-enyl-, 3-methylbut-3-enyl-, 2-methylbut-3-enyl-, 1-methylbut-3-enyl-, 3-methylbut-2-enyl-, (E)-2-methylbut-2-enyl-, (Z)-2-methylbut-2-enyl-, (E)-1-methylbut-2-enyl-, (Z)-1-methylbut-2-enyl-, (E)-3-methylbut-1-enyl-, (Z)-3-methylbut-1-enyl-, (E)-2-methylbut-1-enyl-, (Z)-2-methylbut-1-enyl-, (E)-1-methylbut-1-enyl-, (Z)-1-methylbut-1-enyl-, 1,1-dimethylprop-2-enyl-, 1-ethylprop-1-enyl-, 1-propylvinyl-, 1-isopropylvinyl-4-methylpent-4-enyl-, 3-methylpent-4-enyl-, 2-methylpent-4-enyl-, 1-methylpent-4-enyl-, 4-methylpent-3-enyl-, (E)-3-methylpent-3-enyl-, (Z)-3-methylpent-3-enyl-, (E)-2-methylpent-3-enyl-, (Z)-2-methylpent-3-enyl-, (E)-1-methylpent-3-enyl-, (Z)-1-methylpent-3-enyl-, (E)-4-methylpent-2-enyl-, (Z)-4-methylpent-2-enyl-, (E)-3-methylpent-2-enyl-, (Z)-3-methylpent-2-enyl-, (E)-2-methylpent-2-enyl-, (Z)-2-methylpent-2-enyl-, (E)-1-methylpent-2-enyl-, (Z)-1-methylpent-2-enyl-, (E)-4-methylpent-1-enyl-, (Z)-4-methylpent-1-enyl-, (E)-3-methylpent-1-enyl-, (Z)-3-methylpent-1-enyl-, (E)-2-methylpent-1-enyl-, (Z)-2-methylpent-1-enyl-, (E)-1-methylpent-1-enyl-, (Z)-1-methylpent-1-enyl-, 3-ethylbut-3-enyl-, 2-ethylbut-3-enyl-, 1-ethylbut-3-enyl-, (E)-3-ethylbut-2-enyl-, (Z)-3-ethylbut-2-enyl-, (E)-2-ethylbut-2-enyl-, (Z)-2-ethylbut-2-enyl-, (E)-1-ethylbut-2-enyl-, (Z)-1-ethylbut-2-enyl-, (E)-3-ethylbut-1-enyl-, (Z)-3-ethylbut-1-enyl-, 2-ethylbut-1-enyl-, (E)-1-ethylbut-1-enyl-, (Z)-1-ethylbut-1-enyl-, 2-propylprop-2-enyl-, 1-propylprop-2-enyl-, 2-isopropylprop-2-enyl-, 1-isopropylprop-2-enyl-, (E)-2-propylprop-1-enyl-, (Z)-2-propylprop-1-enyl-, (E)-1-propylprop-1-enyl-, (Z)-1-propylprop-1-enyl-, (E)-2-isopropylprop-1-enyl-, (Z)-2-isopropylprop-1-enyl-, (E)-1-isopropylprop-1-enyl-, (Z)-1-isopropylprop-1-enyl-, (E)-3,3-dimethylprop-1-enyl-, (Z)-3,3-dimethylprop-1-enyl-, 1-(1,1-dimethylethyl)ethenyl-, buta-1,3-dienyl-, penta-1,4-dienyl-, hexa-1,5-dienyl-, or methylhexadienyl- group. Particularly, said group is vinyl- or allyl-.

The term “C₂-C₆-alkynyl-” is to be understood as preferably meaning a linear or branched, monovalent hydrocarbon group which contains one or more triple bonds, and which contains 2, 3, 4, 5 or 6 carbon atoms, particularly 3, 4, 5 or 6 carbon atoms (“C₃-C₆-alkynyl-”), more particularly 3 or 4 carbon atoms (“C₃-C₄-alkynyl-”). Said C₂-C₆-alkynyl- group is, for example, ethynyl-, prop-1-ynyl-, prop-2-ynyl-, but-1-ynyl-, but-2-ynyl-, but-3-ynyl-, pent-1-ynyl-, pent-2-ynyl-, pent-3-ynyl-, pent-4-ynyl-, hex-1-ynyl-, hex-2-ynyl-, hex-3-ynyl-, hex-4-ynyl-, hex-5-ynyl-, 1-methylprop-2-ynyl-, 2-methylbut-3-ynyl-, 1-methylbut-3-ynyl-, 1-methylbut-2-ynyl-, 3-methylbut-1-ynyl-, 1-ethylprop-2-ynyl-, 3-methylpent-4-ynyl-, 2-methylpent-4-ynyl-, 1-methylpent-4-ynyl-, 2-methylpent-3-ynyl-, 1-methylpent-3-ynyl-, 4-methylpent-2-ynyl-, 1-methylpent-2-ynyl-, 4-methylpent-1-ynyl-, 3-methylpent-1-ynyl-, 2-ethylbut-3-ynyl-, 1-ethylbut-3-ynyl-, 1-ethylbut-2-ynyl-, 1-propylprop-2-ynyl-, 1-isopropylprop-2-ynyl-, 2,2-dimethylbut-3-ynyl-, 1,1-dimethylbut-3-ynyl-, 1,1-dimethylbut-2-ynyl-, or 3,3-dimethylbut-1-ynyl- group. Particularly, said alkynyl- group is ethynyl-, prop-1-ynyl-, or prop-2-ynyl-.

The term “C₃-C₇-cycloalkyl-” is to be understood as meaning a saturated, monovalent, monocyclic hydrocarbon ring which contains 3, 4, 5, 6 or 7 carbon atoms. Said C₃-C₇-cycloalkyl- group is for example a cyclopropyl-, cyclobutyl-, cyclopentyl-, cyclohexyl- or cycloheptyl- ring. Particularly, said ring contains 3, 4, 5 or 6 carbon atoms (“C₃-C₆-cycloalkyl-”), more particularly, said ring contains 5 or 6 carbon atoms (“C₅-C₆-cycloalkyl-”).

The term “C₄-C₈-cycloalkenyl-” is to be understood as preferably meaning a monovalent, monocyclic hydrocarbon ring which contains 4, 5, 6, 7 or 8 carbon atoms and one or two double bonds, in conjugation or not, as the size of said cycloalkenyl- ring allows. Particularly, said ring contains 4, 5 or 6 carbon atoms (“C₄-C₆-cycloalkenyl-”). Said C₄-C₈-cycloalkenyl- group is for example a cyclobutenyl-, cyclopentenyl-, or cyclohexenyl- group.

The term “4- to 10-membered heterocycloalkyl-” is to be understood as meaning a saturated, monovalent, mono- or bicyclic hydrocarbon ring which contains 3, 4, 5, 6, 7, 8 or 9 carbon atoms, and one or more heteroatom-containing groups selected from —O—, —S—, —S(═O)—, —S(═O)₂—, —NR^{a}—, in which R^{a} represents a hydrogen atom or a C₁-C₆-alkyl- or C₃-C₇-cycloalkyl-group; it being possible for said heterocycloalkyl- group to be attached to the rest of the molecule via any one of the carbon atoms or, if present, a nitrogen atom. Heterospirocycloalkyl-, heterobicycloalkyl- and bridged heterocycloalkyl-, as defined infra, are also included within the scope of this definition.

The term “heterospirocycloalkyl-” is to be understood as meaning a saturated, monovalent bicyclic hydrocarbon radical in which the two rings share one common ring carbon atom, and wherein said bicyclic hydrocarbon radical contains 3, 4, 5, 6, 7, 8 or 9 carbon atoms, and one or more heteroatom-containing groups selected from —O—, —S—, —S(═O)—, —S(═O)₂—, —NR^{a}—, in which R^{a} represents a hydrogen atom or a C₁-C₆-alkyl- or C₃-C₇-cycloalkyl-group; it being possible for said heterospirocycloalkyl- group to be attached to the rest of the molecule via any one of the carbon atoms or, if present, a nitrogen atom. Said heterospirocycloalkyl- group is, for example, azaspiro[2.3]hexyl-, azaspiro[3.3]heptyl-, oxaazaspiro[3.3]heptyl-, thiaazaspiro[3.3]heptyl-, oxaspiro[3.3]heptyl-, oxazaspiro[5.3]nonyl-, oxazaspiro[4.3]octyl-, oxazaspiro[5.5]undecyl-, diazaspiro[3.3]heptyl-, thiazaspiro[3.3]heptyl-, thiazaspiro[4.3]octyl-, or azaspiro[5.5]decyl-.

The term “heterobicycloalkyl-” is to be understood as meaning a saturated, monovalent bicyclic hydrocarbon radical in which the two rings share two immediately adjacent ring atoms, and wherein said bicyclic hydrocarbon radical contains 3, 4, 5, 6, 7, 8 or 9 carbon atoms, and one or more heteroatom-containing groups selected from —O—, —S—, —S(═O)—, —S(═O)₂—, —NR^{a}—, in which R^{a} represents a hydrogen atom or a C₁-C₆-alkyl- or C₃-C₇-cycloalkyl-group; it being possible for said heterobicycloalkyl- group to be attached to the rest of the molecule via any one of the carbon atoms or, if present, a nitrogen atom. Said heterobicycoalkyl- group is, for example, azabicyclo[3.3.0]octyl-, azabicyclo[4.3.0]nonyl-, diazabicyclo[4.3.0]nonyl-, oxazabicyclo[4.3.0]nonyl-, thiazabicyclo[4.3.0]nonyl-, or azabicyclo[4.4.0]decyl-.

The term “bridged heterocycloalkyl-” is to be understood as meaning a saturated, monovalent bicyclic hydrocarbon radical in which the two rings share two common ring atoms which are not immediately adjacent, and wherein said bicyclic hydrocarbon radical contains 3, 4, 5, 6, 7, 8 or 9 carbon atoms, and one or more heteroatom-containing groups selected from —O—, —S—, —S(═O)—, —S(═O)₂—, —NR^{a}—, in which R^{a} represents a hydrogen atom, or a C₁-C₆-alkyl- or C₃-C₇-cycloalkyl- group; it being possible for said bridged heterocycloalkyl- group to be attached to the rest of the molecule via any one of the carbon atoms or, if present, a nitrogen atom. Said bridged heterocycloalkyl- group is, for example, azabicyclo[2.2.1]heptyl-, oxazabicyclo[2.2.1]heptyl-, thiazabicyclo[2.2.1]heptyl-, diazabicyclo[2.2.1]heptyl-, azabicyclo[2.2.2]octyl-, diazabicyclo[2.2.2]octyl-, oxazabicyclo[2.2.2]octyl-, thiazabicyclo[2.2.2]octyl-, azabicyclo[3.2.1]octyl-, diazabicyclo[3.2.1]octyl-, oxazabicyclo[3.2.1]octyl-, thiazabicyclo[3.2.1]octyl-, azabicyclo[3.3.1]nonyl-, diazabicyclo[3.3.1]nonyl-, oxazabicyclo[3.3.1]nonyl-, thiazabicyclo[3.3.1]nonyl-, azabicyclo[4.2.1]nonyl-, diazabicyclo[4.2.1]nonyl-, oxazabicyclo[4.2.1]nonyl, thiazabicyclo[4.2.1]nonyl-, azabicyclo[3.3.2]decyl-, diazabicyclo[3.3.2]decyl-, oxazabicyclo[3.3.2]decyl-, thiazabicyclo[3.3.2]decyl-, or azabicyclo[4.2.2]decyl-.

Particularly, said 4- to 10-membered heterocycloalkyl- can contain 3, 4, 5 or 6 carbon atoms, and one or more of the above-mentioned heteroatom-containing groups (a “4- to 7-membered heterocycloalkyl-”), more particularly said heterocycloalkyl- can contain 4 or 5 carbon atoms, and one or more of the above-mentioned heteroatom-containing groups (a “5- to 6-membered heterocycloalkyl-”).

In a preferred embodiment, the 5- to 6-membered heterocycloalkyl- group is a piperidinyl- group.

Particularly, without being limited thereto, said heterocycloalkyl- can be a 4-membered ring, such as an azetidinyl-, oxetanyl-, or a 5-membered ring, such as tetrahydrofuranyl-, pyrrolidinyl-, imidazolidinyl-, pyrazolidinyl-, or a 6-membered ring, such as tetrahydropyranyl-, piperidinyl-, morpholinyl-, dithianyl-, thiomorpholinyl-, piperazinyl-, or trithianyl-, or a 7-membered ring, such as a diazepanyl- ring, for example.

The term “4- to 10-membered heterocycloalkenyl-”, is to be understood as meaning an unsaturated, monovalent, mono- or bicyclic hydrocarbon ring which contains 3, 4, 5, 6, 7, 8 or 9 carbon atoms, and one or more heteroatom-containing groups selected from —O—, —S—, —S(═O)—, —S(═O)₂—, —NR^{a}—, in which R^{a} represents a hydrogen atom or a C₁-C₆-alkyl- group; it being possible for said heterocycloalkenyl- group to be attached to the rest of the molecule via any one of the carbon atoms or, if present, a nitrogen atom. Examples of said heterocycloalkenyl- may contain one or more double bonds, e.g. 4H-pyranyl-, 2H-pyranyl-, 2,5-dihydro-1H-pyrrolyl-, 4H-[1,3,4]thiadiazinyl-, 2,5-dihydrofuranyl-, 2,3-dihydrofuranyl-, 2,5-dihydrothiophenyl-, 2,3-dihydrothiophenyl-, 4,5-dihydrooxazolyl-, or 4H-[1,4]thiazinyl- group.

The term “aryl-” is to be understood as preferably meaning a monovalent, aromatic, mono-, or bi- or tricyclic hydrocarbon ring system having 6, 7, 8, 9, 10, 11, 12, 13 or 14 carbon atoms (a “C₆-C₁₄-aryl-” group), particularly a group having 6 carbon atoms (a “C₆-aryl-” group), e.g. a phenyl- group; or a group having 9 carbon atoms (a “C₉-aryl-” group), e.g. an indanyl- or indenyl- group, or a group having 10 carbon atoms (a “C₁₀-aryl-” group), e.g. a tetralinyl-, dihydronaphthyl-, or naphthyl- group, or a biphenyl- group (a “C₁₂-aryl-” group), or a group having 13 carbon atoms, (a “C₁₃-aryl-” group), e.g. a fluorenyl- group, or a group having 14 carbon atoms, (a “C₁₄-aryl-” group), e.g. an anthracenyl- group. Preferably, the aryl- group is a phenyl- group.

The term “heteroaryl-” is understood as preferably meaning an “aryl-” group as defined supra, in which at least one of the carbon ring atoms is replaced by a heteroatom selected from oxygen, nitrogen, and sulphur. The “heteroaryl-” group contains 5, 6, 7, 8, 9, 10, 11, 12, 13 or 14 ring atoms (a “5- to 14-membered heteroaryl-” group), particularly 5 or 6 or 9 or 10 ring atoms (a “5- to 10-membered heteroaryl-” group), more particularly 5 or 6 ring atoms (a “5- to 6-membered heteroaryl-” group). Particularly, heteroaryl- is selected from thienyl-, furanyl-, pyrrolyl-, oxazolyl-, thiazolyl-, imidazolyl-, pyrazolyl-, isoxazolyl-, isothiazolyl-, oxadiazolyl-, triazolyl-, thiadiazolyl-, thia-4H-pyrazolyl- etc., and benzo derivatives thereof, such as, for example, benzofuranyl-, benzothienyl-, benzoxazolyl-, benzisoxazolyl-, benzimidazolyl-, benzotriazolyl-, benzothiadiazolyl-, indazolyl-, indolyl-, isoindolyl-, etc.; or pyridinyl-, pyridazinyl-, pyrimidinyl-, pyrazinyl-, triazinyl-, etc., and benzo derivatives thereof, such as, for example, quinolinyl-, quinazolinyl-, isoquinolinyl-, etc.; or azocinyl-, indolizinyl-, purinyl-, etc., and benzo derivatives thereof; or cinnolinyl-, phthalazinyl-, quinazolinyl-, quinoxalinyl-, naphthpyridinyl-, pteridinyl-, carbazolyl-, acridinyl-, phenazinyl-, phenothiazinyl-, phenoxazinyl-, xanthenyl-, or oxepinyl-, etc.

In a preferred embodiment, the heteroaryl- group is selected from: pyridyl, oxazolyl, pyrazolyl, thiazolyl, and oxadiazolyl.

In general, and unless otherwise mentioned, the heteroarylic or heteroarylenic radicals include all the possible isomeric forms thereof, e.g. the positional isomers thereof. Thus, for some illustrative non-restricting example, the term pyridyl- includes pyridin-2-yl-, pyridin-3-yl-, and pyridin-4-yl-; or the term thienyl- includes thien-2-yl- and thien-3-yl-. Preferably, the heteroaryl- group is a pyridinyl- group.

The term “C₁-C₆”, as used throughout this text, e.g. in the context of the definition of “C₁-C₆-alkyl-” is to be understood as meaning an alkyl- group having a finite number of carbon atoms of 1 to 6, i.e. 1, 2, 3, 4, 5, or 6 carbon atoms. It is to be understood further that said term “C₁-C₆” is to be interpreted as any sub-range comprised therein, e.g. C₁-C₆, C₂-C₅, C₃-C₄, C₁-C₂, C₁-C₃, C₁-C₄, C₁-C₅, C₁-C₆; particularly C₁-C₂, C₁-C₃, C₁-C₄, C₁-C₅, C₁-C₆; more particularly C₁-C₄; in the case of “C₁-C₃-haloalkyl-” or “halo-C₁-C₃-alkoxy-” even more particularly C₁-C₂.

Similarly, as used herein, the term “C₂-C₆”, as used throughout this text, e.g. in the context of the definitions of “C₂-C₆-alkenyl-” and “C₂-C₆-alkynyl-”, is to be understood as meaning an alkenyl- group or an alkynyl group having a finite number of carbon atoms of 2 to 6, i.e. 2, 3, 4, 5, or 6 carbon atoms. It is to be understood further that said term “C₂-C₆” is to be interpreted as any sub-range comprised therein, e.g. C₂-C₆, C₃-C₅, C₃-C₄, C₂-C₃, C₂-C₄, C₂-C₅; particularly C₂-C₃.

Further, as used herein, the term “C₃-C₇”, as used throughout this text, e.g. in the context of the definition of “C₃-C₇-cycloalkyl”, is to be understood as meaning a cycloalkyl group having a finite number of carbon atoms of 3 to 7, i.e. 3, 4, 5, 6 or 7 carbon atoms. It is to be understood further that said term “C₃-C₇” is to be interpreted as any sub-range comprised therein, e.g. C₃-C₆, C₄-C₅, C₃-C₅, C₃-C₄, C₄-C₆, C₅-C₇; particularly C₃-C₆.

As used herein, the term “Leaving group” refers to an atom or a group of atoms that is displaced in a chemical reaction as stable species taking with it the bonding electrons. The leaving group as used herein is suitable for nucleophilic aliphatic and/or aromatic substitution, e.g. a halogen atom, in particular chloro-, bromo- or iodo-, or a group selected from methanesulfonyloxy-, p-toluenesulfonyloxy-, trifluoromethanesulfonyloxy-, nonafluorobutanesulfonyloxy-, (4-bromo-benzene)sulfonyloxy-, (4-nitro-benzene)sulfonyloxy-, (2-nitro-benzene)-sulfonyloxy-, (4-isopropyl-benzene)sulfonyloxy-, (2,4,6-tri-isopropyl-benzene)-sulfonyloxy-, (2,4,6-trimethyl-benzene)sulfonyloxy-, (4-tert-butyl-benzene)sulfonyloxy-, benzenesulfonyloxy-, and (4-methoxy-benzene)sulfonyloxy-.

As used herein, the term “protective group” is a protective group attached to a nitrogen in intermediates used for the preparation of compounds of the general formula (I). Such groups are introduced e.g. by chemical modification of the respective amino group in order to obtain chemoselectivity in a subsequent chemical reaction. Protective groups for amino groups are described for example in T. W. Greene and P. G. M. Wuts in Protective Groups in Organic Synthesis, 3^{rd} edition, Wiley 1999; more specifically, said groups can be selected from substituted sulfonyl groups, such as mesyl-, tosyl- or phenylsulfonyl-, acyl groups such as benzoyl-, acetyl- or tetrahydropyranoyl-, or carbamate based groups, such as tert.-butoxycarbonyl- (Boc), or can include silicon, as in e.g. 2-(trimethylsilyl)ethoxymethyl- (SEM).

As used herein, the term “one or more times”, e.g. in the definition of the substituents of the compounds of the general formulae of the present invention, is understood as meaning “one, two, three, four or five times, particularly one, two, three or four times, more particularly one, two or three times, even more particularly one or two times”.

Where the plural form of the word compounds, salts, polymorphs, hydrates, solvates and the like, is used herein, this is taken to mean also a single compound, salt, polymorph, isomer, hydrate, solvate or the like.

The compounds of this invention contain one or more asymmetric centres, depending upon the location and nature of the various substituents desired.

Asymmetric carbon atoms may be present in the (R) or (S) configuration. In certain instances, asymmetry may also be present due to restricted rotation about a given bond, for example, the central bond adjoining two substituted aromatic rings of the specified compounds.

Substituents on a ring may also be present in either cis or trans form. It is intended that all such configurations are included within the scope of the present invention.

Preferred compounds are those which produce the more desirable biological activity. Separated, pure or partially purified isomers and stereoisomers or racemic or diastereomeric mixtures of the compounds of this invention are also included within the scope of the present invention. The purification and the separation of such materials can be accomplished by standard techniques known in the art.

The optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Diacel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976).

The invention also includes all suitable isotopic variations of a compound of the invention. An isotopic variation of a compound of the invention is defined as one in which at least one atom is replaced by an atom having the same atomic number but an atomic mass different from the atomic mass usually or predominantly found in nature. Examples of isotopes that can be incorporated into a compound of the invention include isotopes of hydrogen, carbon, nitrogen, oxygen, phosphorus, sulphur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁹I and ¹³¹I, respectively. Certain isotopic variations of a compound of the invention, for example, those in which one or more radioactive isotopes such as ³H or ¹⁴C are incorporated, are useful in drug and/or substrate tissue distribution studies. Tritiated and carbon-14, i.e., ¹⁴C, isotopes are particularly preferred for their ease of preparation and detectability. Further, substitution with isotopes such as deuterium may afford certain therapeutic advantages resulting from greater metabolic stability, for example, increased in vivo half-life or reduced dosage requirements and hence may be preferred in some circumstances. Isotopic variations of a compound of the invention can generally be prepared by conventional procedures known by a person skilled in the art such as by the illustrative methods or by the preparations described in the examples hereafter using appropriate isotopic variations of suitable reagents.

The present invention includes all possible stereoisomers of the compounds of the present invention as single stereoisomers, or as any mixture of said stereoisomers, in any ratio. Isolation of a single stereoisomer, e.g. a single enantiomer or a single diastereomer, of a compound of the present invention may be achieved by any suitable state of the art method, such as chromatography, especially chiral chromatography, for example.

Further, the compounds of the present invention may exist as tautomers. For example, any compound of the present invention which contains a pyrazole moiety as a heteroaryl group for example can exist as a 1H tautomer, or a 2H tautomer, or even a mixture in any amount of the two tautomers, or a triazole moiety for example can exist as a 1H tautomer, a 2H tautomer, or a 4H tautomer, or even a mixture in any amount of said 1H, 2H and 4H tautomers, viz.

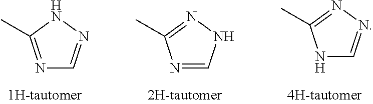

The present invention includes all possible tautomers of the compounds of the present invention as single tautomers, or as any mixture of said tautomers, in any ratio.

Further, the compounds of the present invention can exist as N-oxides, which are defined in that at least one nitrogen of the compounds of the present invention is oxidised. The present invention includes all such possible N-oxides.

The present invention also relates to useful forms of the compounds as disclosed herein, such as metabolites, hydrates, solvates, prodrugs, salts, in particular pharmaceutically acceptable salts, and co-precipitates.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19.

The present invention includes all possible salts of the compounds of the present invention as single salts, or as any mixture of said salts, in any ratio.

Furthermore, the present invention includes all possible crystalline forms, or polymorphs, of the compounds of the present invention, either as single polymorphs, or as a mixture of more than one polymorphs, in any ratio.

In accordance with a first aspect, the present invention relates to compounds of general formula (I):

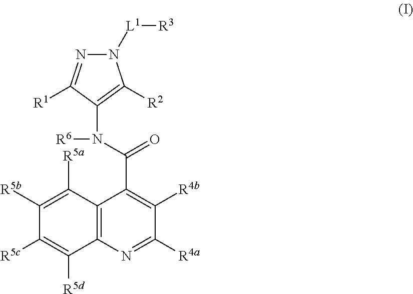

in which:
R¹ represents a hydrogen atom;
R² represents a C₁-C₃-alkyl-, halo-C₁-C₃-alkyl-, cyano-, —C(═O)O—R¹⁰ or —C(═O)N(R^{10a})R^{10b} group;
R³ represents a group selected from: phenyl-, heteroaryl-, C₅-C₆-cycloalkyl-, and 5- to 6-membered heterocycloalkyl-;
   wherein said 5- to 6-membered heterocycloalkyl- group is optionally benzocondensed;
   wherein said phenyl-, heteroaryl-, C₅-C₆-cycloalkyl-, and 5- to 6-membered heterocycloalkyl- group is optionally substituted, one or more times, identically or differently, with -(L²)ₚ-R⁷;
   and wherein two -(L²)ₚ-R⁷ groups, if being present ortho to each other on an aryl- or heteroaryl- group optionally form a bridge selected from: *—C₃-C₅-alkylene-*, *—O(CH₂)₂O—*, *—O(CH₂)O—*, *—O(CF₂)O—*, *—CH₂C(R^{10a})(R^{10b})O—*, *—C(═O)N(R^{10a})CH₂—*, *—N(R^{10a})C(═O)CH₂O—*, *—NHC(═O)NH—*; wherein each * represents the point of attachment to said aryl- or heteroaryl- group;
R^{4a} represents a hydrogen atom or a halogen atom or a group selected from: cyano-, hydroxy-, C₁-C₃-alkyl-, halo-C₁-C₃-alkyl-, C₁-C₃-alkoxy-, C₃-C₇-cycloalkyl-, 4- to 7-membered heterocycloalkyl-, —C(═O)N(R^{10a})R^{10b}, —N(R^{10a})R^{10b};
R^{4b} represents a hydrogen atom or a group selected from: C₁-C₃-alkoxy-, C₁-C₃-alkyl-, cyano-;
or
R^{4a} and together R^{4b} form a —C₃-C₅-alkylene- group;
R^{5a}, R^{5b}, R^{5c}, R^{5d}
   independently from each other represent a hydrogen atom, a halogen atom or a group selected from:
   cyano-, —NO₂, C₁-C₃-alkyl-, halo-C₁-C₃-alkyl-, C₁-C₃-alkoxy-, halo-C₁-C₃-alkoxy-, phenyl-,
   heteroaryl-, —C(═O)R¹⁰, —C(═O)N(H)R¹⁰, —C(═O)N(R^{10a})R^{10b}, —C(═O)O—R¹⁰, —N(R^{10a})R^{10b}, —N(H)C(═O)R¹⁰, —N(R^{10a})C(═O)R^{10b}, —N(H)C(═O)N(R^{10a})R^{10b}, —N(R^{10a})C(═O)N(R^{10b})R^{10c}, —N(R^{10a})C(═O)C(═O)N(R^{10b})R^{10c}, —N(H)C(═O)OR¹⁰, —N(R^{10a})C(═O)OR^{10b}, —N(H)S(═O)₂R¹⁰, —N(R^{10a})S(═O)₂R^{10b}, —OR¹⁰, —O(C═O)R¹⁰, —O(C═O)N(R^{10a})R^{10b}, —O(C═O)OR¹⁰, —SR¹⁰, —S(═O)R¹⁰, —S(═O)₂R¹⁰, —S(═O)₂N(H)R¹⁰, —S(═O)₂N(R^{10a})R^{10b} or —S(═O)(═NR^{10a})R^{10b} said phenyl- or heteroaryl- group being optionally substituted one or more times, identically or differently, with a group selected from: halo-, cyano-, C₁-C₃-alkyl-, halo-C₁-C₃-alkyl-, C₁-C₃-alkoxy- group;
R⁶ represents a hydrogen atom or group selected from: C₁-C₃-alkyl-, C₁-C₃-alkoxy-(L²)-, hydroxy-C₁-C₃-alkyl-, aryl-(L²)-, heteroaryl-(L²)-;
R⁷ represents a group selected from: oxo, C₁-C₃-alkyl-, C₃-C₇-cycloalkyl-, 4- to 7-membered heterocycloalkyl-, halo-C₁-C₃-alkyl-, C₁-C₃-alkoxy-, halo-C₁-C₃-alkoxy-, —OH, —CN, halo-, —C(═O)R⁸, —C(═O)—O—R⁸, —C(═O)N(R^{8a})R^{8b}, —S(═O)₂R⁸, —S(═O)(═N)R¹¹, phenyl-, 5- to 6-membered heteroaryl-;
R⁸ represents a hydrogen atom or a C₁-C₆-alkyl-, halo-C₁-C₃-alkyl-, cyano-C₁-C₄-alkyl-, C₁-C₃-alkoxy-C₁-C₃-alkyl-, C₃-C₇-cycloalkyl-, phenyl-, 5- to 6-membered heteroaryl- or benzyl- group;
R^{8a}, R^{8b}
   represent, independently from each other, a hydrogen atom, or a C₁-C₁₀-alkyl-, C₃-C₇-cycloalkyl-, (C₃-C₇-cycloalkyl)-(L³)-, C₃-C₆-alkenyl-, C₃-C₆-alkynyl-, 4- to 10-membered heterocycloalkyl-, (4- to 10-membered heterocycloalkyl)-(L³)-, phenyl-, heteroaryl-, phenyl-(L³)-, (phenyl)-O-(L³)-, heteroaryl-(L³)-, or (aryl)-(4- to 10-membered heterocycloalkyl)- group;
   said C₁-C₁₀-alkyl-, C₃-C₇-cycloalkyl-, (C₃-C₇-cycloalkyl)-(L³)-, C₃-C₆-alkenyl-, C₃-C₆-alkynyl-, 4- to 10-membered heterocycloalkyl-, (4- to 10-membered heterocycloalkyl)-(L³)-, phenyl-, heteroaryl-, phenyl-(L³)-, (phenyl)-O-(L³)-, heteroaryl-(L³)-, and (aryl)-(4- to 10-membered heterocycloalkyl)- group being optionally substituted one or more times, identically or differently, with R⁹;
or
R^{8a} and R^{8b}, together with the nitrogen atom they are attached to, represent a 4- to 10-membered heterocycloalkyl-group, said 4- to 10-membered heterocycloalkyl-group being optionally substituted one or more times, identically or differently, with R⁹;
R⁹ represents a halogen atom, or a oxo, C₁-C₃-alkyl-, halo-C₁-C₃-alkyl-, hydroxy-C₁-C₃-alkyl-, —CN, —C(═O)R¹⁰, —C(═O)N(H)R¹⁰, —C(═)N(R^{10a})R^{10b}, —C(═O)O—R¹⁰, —N(R^{10a})R^{10b}, —NO₂, —N(H)C(═O)R¹⁰, —N(R^{10a})C(═O)R^{10b}, —N(H)C(═O)N(R^{10a}) R^{10b}, —N(R^{10a})C(═O)N(R^{10b})R^{10c}, —N(H)C(═O)OR¹⁰, —N(R^{10a})C(═O)OR^{10b}, —N(H)S(═O)₂R¹⁰, —N(R^{10a})S(═O)₂R^{10b}, —OR¹⁰, —O(C═O)R¹⁰, —O(C═O)N(R^{10a})R^{10b}, —O(C═O)OR¹⁰, —SR¹⁰, —S(═O)R¹⁰, —S(═O)₂R¹⁰, —S(═O)₂N(H)R¹⁰, —S(═O)₂N(R^{10a})R^{10b}, —S(═O)(=NR^{10a})R^{10b} or a tetrazolyl- group;
or
   two R⁹ groups present ortho to each other on a phenyl- or heteroaryl-ring form a bridge selected from: *—C₃-C₅-alkylene-*, *—O(CH₂)₂O—*, *—O(CH₂)O—*, *—O(CF₂)O—*, *—CH₂C(R^{10a})(R^{10b})O—*, *—C(═O)N(R^{10a})CH₂—*, *—N(R^{10a})C(═O)CH₂O—*, *—NHC(═O)NH—*; wherein each * represents the point of attachment to said phenyl- or heteroaryl- ring;
R¹⁰, R^{10a}, R^{10b}, R^{10c}
   represent, independently from each other, a hydrogen atom or a group selected from: C₁-C₃-alkyl-, halo-C₁-C₃-alkyl-, hydroxy-C₁-C₃-alkyl-, C₁-C₃-alkoxy-C₁-C₃-alkyl-, C₃-C₇-cycloalkyl-;
R¹¹ represents a hydrogen atom or a cyano-, C₁-C₃-alkyl-, —C(═O)R¹⁰, —C(═O)N(H)R¹⁰, —C(═O)N(R^{10a})R^{10b} or —C(═O)O—R¹⁰ group;
L¹ represents a group selected from: —C₁-C₄-alkylene-, —CH₂—CH═CH—, —C(phenyl)(H)—, —CH₂—CH₂—O—;
L² represents a group selected from: —CH₂—, —CH₂—CH₂—, —CH₂—CH₂—CH₂—;
L³ represents a —C₁-C₆-alkylene- group;
p is an integer of 0 or 1;
   or a tautomer, a stereoisomer, an N-oxide, a hydrate, a solvate, or a salt thereof, or a mixture of same.

In a preferred embodiment, the invention relates to compounds of formula (I), supra, wherein R² represents a C₁-C₃-alkyl-, halo-C₁-C₃-alkyl-, —C(═O)O—R¹⁰ or —C(═O)N(R^{10a})R^{10b} group.

In a preferred embodiment, the invention relates to compounds of formula (I), supra, wherein R² represents a C₁-C₃-alkyl-, —C(═O)O—R¹⁰ or —C(═O)N(R^{10a})R^{10b} group.

In another preferred embodiment, the invention relates to compounds of formula (I), supra, wherein R² represents a C₁-C₃-alkyl- group.

In another preferred embodiment, the invention relates to compounds of formula (I), supra, wherein R² represents a methyl, ethyl or iso-propyl group.

In another preferred embodiment, the invention relates to compounds of formula (I), supra, wherein R² represents a halo-C₁-C₃-alkyl- group.

In another preferred embodiment, the invention relates to compounds of formula (I), supra, wherein R² represents a —C(═O)O—R¹⁰ group.

In another preferred embodiment, the invention relates to compounds of formula (I), supra, wherein R² represents a —C(═O)OH or —C(═O)OCH₃ group.

In another preferred embodiment, the invention relates to compounds of formula (I), supra, wherein R² represents a —C(═O)N(R^{10a})R^{10b} group.

In another preferred embodiment, the invention relates to compounds of formula (I), supra, wherein R² represents —CH₃, —CH₂—CH₃, —CH(CH₃)₂, —CF₃, —C(═O)—O—CH₃, —C(═O)—OH, —C(═O)—N(CH₃)₂, —C(═O)—NH₂, —C(═O)—N(H)—CH₃, —C(═O)—N(H)—CH₂—CH₂—OH or —C(═O)—N(H)—CH₂—CH₂—O—CH₃.

In another preferred embodiment, the invention relates to compounds of formula (I), supra, wherein R² represents —CH₃, —CF₃, —C(═O)—O—CH₃, —C(═O)—OH, —C(═O)—N(CH₃)₂, —C(═O)—NH₂, —C(═O)—N(H)—CH₃, —C(═O)—N(H)—CH₂—CH₂—OH or —C(═O)—N(H)—CH₂—CH₂—O—CH₃.

In another preferred embodiment, the invention relates to compounds of formula (I), supra, wherein R² represents —CH₃, —C(═O)—O—CH₃, —C(═O)—OH, —C(═O)—NH₂, —C(═O)—N(H)—CH₃.

In another preferred embodiment, the invention relates to compounds of formula (I), supra, wherein R² represents —CH₃ or —CF₃.

In another preferred embodiment, the invention relates to compounds of formula (I), supra, wherein R² represents —CH₃.

In another preferred embodiment, the invention relates to compounds of formula (I), supra, wherein R² represents —CF₃.

In another preferred embodiment, the invention relates to compounds of formula (I), supra, wherein R² represents a C₁-C₃-alkyl-, halo-C₁-C₃-alkyl- or cyano- group.

In another preferred embodiment, the invention relates to compounds of formula (I), supra, wherein R³ represents a phenyl- or 5- to 6-membered heteraryl- group;

wherein said phenyl- and 5- to 6-membered heteraryl- group is optionally substituted, one or more times, identically or differently, with -(L²)ₚ-R⁷.

In another preferred embodiment, the invention relates to compounds of formula (I), supra, wherein R³ represents a phenyl- or pyridyl- group; wherein said phenyl- and pyridyl- group is optionally substituted, one or more times, identically or differently, with -(L²)ₚ-R⁷.

In another preferred embodiment, the invention relates to compounds of formula (I), supra, wherein R³ represents a phenyl- or pyridyl- group; wherein said phenyl- group is optionally substituted, one or more times, identically or differently, with -(L²)ₚ-R⁷.

In another preferred embodiment, the invention relates to compounds of formula (I), supra, wherein R³ represents a phenyl- or pyridyl- group; wherein said phenyl- group is optionally substituted, one or more times, identically or differently, with -(L²)ₚ-R⁷, in which p is an integer 0, and in which R⁷ represents a halogen atom, or represents a group selected from C₁-C₃-alkyl-, —CN, C₁-C₃-alkoxy-, —C(═O)N(R^{8a})R^{8b} and —S(═O)₂R⁸.

In another preferred embodiment, the invention relates to compounds of formula (I), supra, wherein R³ represents a pyridyl- group.

In another preferred embodiment, the invention relates to compounds of formula (I), supra, wherein R³ represents a cyclohexyl- group.

In another preferred embodiment, the invention relates to compounds of formula (I), supra, wherein R³ represents a piperidinyl- group; wherein said group is optionally substituted one time with —S(═O)₂—CH₂—CH₃.

In another preferred embodiment, the invention relates to compounds of formula (I), supra, wherein R³ represents a heteroaryl- group which is selected from: pyridyl, oxazolyl, pyrazolyl, thiazolyl, oxadiazolyl; wherein said heteroaryl- group is optionally substituted one time with -(L²)ₚ-R⁷.

In another preferred embodiment, the invention relates to compounds of formula (I), supra, wherein R³ represents a heteroaryl- group which is selected from: pyridyl-, isoxazolyl-, pyrazolyl-, thiazolyl-, oxadiazolyl-; wherein said heteroaryl- group is optionally substituted one time with -(L²)ₚ-R⁷.

In another preferred embodiment, the invention relates to compounds of formula (I), supra, wherein R³ represents a oxadiazolyl- group; wherein said group is optionally substituted one time with —C(═O)—N(H)CH₃.

In another preferred embodiment, the invention relates to compounds of formula (I), supra, wherein R³ represents a thiazolyl- group; wherein said group is optionally substituted one time with methyl.

In another preferred embodiment, the invention relates to compounds of formula (I), supra, wherein R³ represents a pyrazolyl- group; wherein said group is optionally substituted one time with methyl.

In another preferred embodiment, the invention relates to compounds of formula (I), supra, wherein R³ represents a oxazolyl- group; wherein said group is optionally substituted one time with methyl.

In another preferred embodiment, the invention relates to compounds of formula (I), supra, wherein R³ represents an isoxazolyl- group; wherein said group is optionally substituted one time with methyl.

In another preferred embodiment, the invention relates to compounds of formula (I), supra, wherein R³ represents a phenyl- group;

wherein said phenyl- group is optionally substituted one time with -(L²)ₚ-R⁷.

In another preferred embodiment, the invention relates to compounds of formula (I), supra, wherein R³ represents a phenyl- group; wherein said phenyl- group is optionally substituted, one or two times, with fluoro.

In another preferred embodiment, the invention relates to compounds of formula (I), supra, wherein R³ represents a phenyl- group; wherein said phenyl- group is optionally substituted, one time, with cyano.

In another preferred embodiment, the invention relates to compounds of formula (I), supra, wherein R³ represents a phenyl- group; wherein said phenyl- group is optionally substituted, one time, with methoxy.

In another preferred embodiment, the invention relates to compounds of formula (I), supra, wherein R³ represents a pyrazolyl- group; wherein said group is optionally substituted with a methyl group.

In another preferred embodiment, the invention relates to compounds of formula (I), supra, wherein R³ represents an oxazolyl- group; wherein said group is optionally substituted with a methyl group.

In another preferred embodiment, the invention relates to compounds of formula (I), supra, wherein R³ represents a thiazolyl- group; wherein said group is optionally substituted with a methyl group.

In another preferred embodiment, the invention relates to compounds of formula (I), supra, wherein R³ represents an oxadiazolyl- group; wherein said group is optionally substituted with a —C(═O)N(H)CH₃ group.

In another preferred embodiment, the invention relates to compounds of formula (I), supra, wherein R³ represents a pyridyl- group.

In another preferred embodiment, the invention relates to compounds of formula (I), supra, wherein R³ represents a cyclohexyl- group.

In another preferred embodiment, the invention relates to compounds of formula (I), supra, wherein R³ represents a piperidinyl- group; wherein said group is optionally substituted with a —S(═O)₂—CH₂—CH₃ group.

In another preferred embodiment, the invention relates to compounds of formula (I), supra, wherein R³ is selected from:

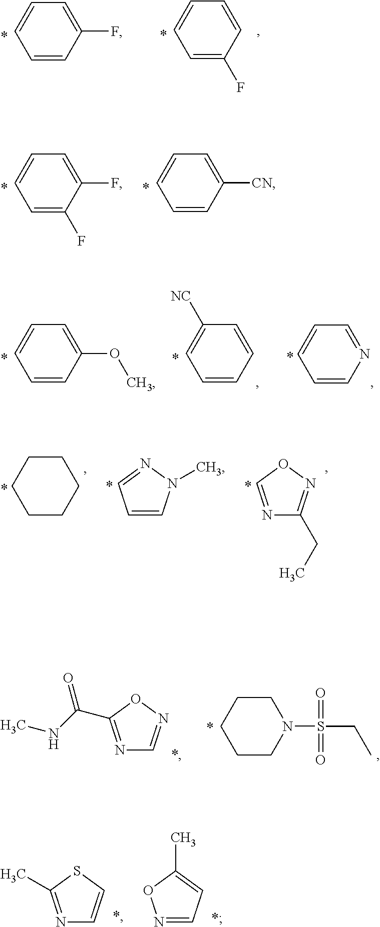

wherein * represents the point of attachment of said groups to L.

In another preferred embodiment, the invention relates to compounds of formula (I), supra, wherein R³ represents a phenyl- group;

wherein said phenyl- group is optionally substituted one or two times, identically or differently, with -(L²)ₚ-R⁷, in which p is an integer 0, and in which R⁷ represents a halogen atom, or represents a group selected from C₁-C₃-alkyl-, —CN, and C₁-C₃-alkoxy-.

In another preferred embodiment, the invention relates to compounds of formula (I), supra, wherein R³ represents a phenyl- group;

wherein said phenyl- group is optionally substituted one or two times, identically or differently, with -(L²)ₚ-R⁷, in which p is an integer 0, and in which R⁷ represents a halogen atom, or represents a group selected from C₁-C₃-alkyl-, —CN, and C₁-C₃-alkoxy, and in which compounds R² represents a methyl- group, R^{4b} represents a hydrogen atom and R⁶ represents a hydrogen atom.

In another preferred embodiment, the invention relates to compounds of formula (I), supra, wherein R³ represents a phenyl- group;

wherein said phenyl- group is substituted once with a fluorine atom or a —CN group.

In another preferred embodiment, the invention relates to compounds of formula (I), supra, wherein R³ represents a phenyl- group;

wherein said phenyl- group is substituted once with a fluorine atom or a —CN group, and in which compounds R² represents a methyl- group, R^{4b} represents a hydrogen atom and R⁶ represents a hydrogen atom.

In another preferred embodiment, the invention relates to compounds of formula (I), supra, wherein R^{4a} represents a halogen atom or a group selected from: cyano-, hydroxy-, C₁-C₃-alkyl-, halo-C₁-C₃-alkyl-, C₁-C₃-alkoxy-, C₃-C₇-cycloalkyl-, 4- to 7-membered heterocycloalkyl-, —C(═O)N(R^{10a})R^{10b}, —N(R^{10a})R^{10b}.

In another preferred embodiment, the invention relates to compounds of formula (I), supra, wherein R^{4a} represents a halogen atom or a group selected from: cyano-, hydroxy-, C₁-C₃-alkyl-, halo-C₁-C₃-alkyl-, C₁-C₃-alkoxy-, C₃-C₇-cycloalkyl-, —C(═O)N(R^{10a})R^{10b}, —N(R^{10a})R^{10b}.

In another preferred embodiment, the invention relates to compounds of formula (I), supra, wherein R^{4a} represents a group selected from: C₁-C₃-alkyl-, halo-C₁-C₃-alkyl-, C₁-C₃-alkoxy-, C₃-C₇-cycloalkyl-, —C(═O)N(R^{10a})R^{10b}, —N(R^{10a})R^{10b}.

In another preferred embodiment, the invention relates to compounds of formula (I), supra, wherein R^{4a} represents a group selected from: C₁-C₃-alkyl-, halo-C₁-C₃-alkyl-, C₁-C₃-alkoxy-, C₃-C₇-cycloalkyl-, —C(═O)N(R^{10a})R^{10b} In another preferred embodiment, the invention relates to compounds of formula (I), supra, wherein R^{4a} represents a group selected from: —CH₃, —CF₃, methoxy-, cyclopropyl-, —C(═O)NH₂.

In another preferred embodiment, the invention relates to compounds of formula (I), supra, wherein R^{4a} represents a hydrogen atom or a halogen atom or a group selected from: C₁-C₃-alkyl-, halo-C₁-C₃-alkyl-, C₁-C₃-alkoxy-, C₃-C₇-cycloalkyl-, —C(═O)N(R^{10a})R^{10b}.

In another preferred embodiment, the invention relates to compounds of formula (I), supra, wherein R^{4a} represents a —C(═O)NH₂ group.

In another preferred embodiment, the invention relates to compounds of formula (I), supra, wherein R^{4a} represents —C(═O)NH₂, and in which compounds R² represents a methyl- group, R^{4b} represents a hydrogen atom and R⁶ represents a hydrogen atom.

In another preferred embodiment, the invention relates to compounds of formula (I), supra, wherein R^{4a} represents a —CF₃ group.

In another preferred embodiment, the invention relates to compounds of formula (I), supra, wherein R^{4a} represents —CF₃, and in which compounds R² represents a methyl- group, R^{4b} represents a hydrogen atom and R⁶ represents a hydrogen atom.

In another preferred embodiment, the invention relates to compounds of formula (I), supra, wherein R^{4a} represents a methoxy group.

In another preferred embodiment, the invention relates to compounds of formula (I), supra, wherein R^{4a} represents a methyl group.

In another preferred embodiment, the invention relates to compounds of formula (I), supra, wherein R^{4a} represents a cyclopropyl group.

In another preferred embodiment, the invention relates to compounds of formula (I), supra, wherein R^{4b} represents a hydrogen atom.

In another preferred embodiment, the invention relates to compounds of formula (I), supra, wherein R^{5a}, R^{5b}, R^{5c}, R^{5d} independently from each other represent a hydrogen atom, a halogen atom or a group selected from: cyano-, —NO₂, C₁-C₃-alkyl-, halo-C₁-C₃-alkyl-, C₁-C₃-alkoxy-, halo-C₁-C₃-alkoxy-, phenyl-, heteroaryl-, —C(═O)R¹⁰, —C(═O)N(H)R¹⁰, —C(═O)N(R^{10a})R^{10b}, —C(═O)O—R¹⁰, —N(R^{10a})R^{10b}, —N(H)C(═O)R¹⁰, —N(R^{10a})C(═O)R^{10b}, —N(R^{10a})C(═O)C(═O)N(R^{10b})R^{10c}, —N(H)S(═O)₂R¹⁰;

said phenyl- or heteroaryl- group being optionally substituted one or more times with a C₁-C₃-alkyl- group.

In another preferred embodiment, the invention relates to compounds of formula (I), supra, wherein R^{5a}, R^{5b}, R^{5c}, R^{5d} independently from each other represent a hydrogen atom, a halogen atom or a group selected from: —NO₂, C₁-C₃-alkyl-, fluoro-C₁-C₃-alkyl-, C₁-C₃-alkoxy-, fluoro-C₁-C₃-alkoxy-, —C(═O)N(R^{10a})R^{10b}, —C(═O)O—R¹⁰, —N(R^{10a})R^{10b}, —N(H)C(═O)R¹⁰, —N(R^{10a})C(═O)R^{10b}.

In another preferred embodiment, the invention relates to compounds of formula (I), supra, wherein R^{5a}, R^{5b}, R^{5c}, R^{5d} independently from each other represents a hydrogen atom, a halogen atom or a group selected from: methyl-, trifluoromethyl-, methoxy-, trifluoromethoxy-, —C(═O)O—R¹⁰, —NH₂, —N(H)C(═O)R¹⁰, and wherein R¹⁰ represents methyl-.

In another preferred embodiment, the invention relates to compounds of formula (I), supra, wherein R^{5a} represents a hydrogen atom.

In another preferred embodiment, the invention relates to compounds of formula (I), supra, wherein R^{5b} represents a hydrogen atom, a halogen atom or a methyl- group.

In another preferred embodiment, the invention relates to compounds of formula (I), supra, wherein R^{5b} represents a hydrogen atom.

In another preferred embodiment, the invention relates to compounds of formula (I), supra, wherein R^{5b} represents a bromine atom or a chlorine atom or a fluorine atom.

In another preferred embodiment, the invention relates to compounds of formula (I), supra, wherein R^{5b} represents a methyl group.

In another preferred embodiment, the invention relates to compounds of formula (I), supra, wherein R^{5b} represents a hydrogen atom, a fluorine atom, a chlorine atom, a bromine atom or a methyl group.

In another preferred embodiment, the invention relates to compounds of formula (I), supra, wherein R^{5b} represents a hydrogen atom, a bromine atom, a chlorine atom, a fluorine atom or a methyl group, and in which compounds R² represents a methyl- group, R^{4b} represents a hydrogen atom and R⁶ represents a hydrogen atom.

In another preferred embodiment, the invention relates to compounds of formula (I), supra, wherein R^{5c} represents a hydrogen atom or a halogen atom.

In another preferred embodiment, the invention relates to compounds of formula (I), supra, wherein R^{5C} represents a hydrogen atom or a fluorine atom.

In another preferred embodiment, the invention relates to compounds of formula (I), supra, wherein R^{5c} represents a hydrogen atom.

In another preferred embodiment, the invention relates to compounds of formula (I), supra, wherein R^{5c} represents a fluorine atom.

In another preferred embodiment, the invention relates to compounds of formula (I), supra, wherein R^{5d} represents a hydrogen atom or a halogen atom.

In another preferred embodiment, the invention relates to compounds of formula (I), supra, wherein R^{5d} represents a hydrogen atom.

In another preferred embodiment, the invention relates to compounds of formula (I), supra, wherein R^{5d} represents a chlorine atom.

In another preferred embodiment, the invention relates to compounds of formula (I), supra, wherein R^{4b} represents a hydrogen atom, R^{5a} represents a hydrogen atom, and R^{5c} represents a hydrogen atom.

In another preferred embodiment, the invention relates to compounds of formula (I), supra, wherein R^{4b} represents a hydrogen atom, R^{5a} represents a hydrogen atom, R^{5C} represents a hydrogen atom, and R^{5d} represents a hydrogen atom.

In another preferred embodiment, the invention relates to compounds of formula (I), supra, wherein R^{4b} represents a hydrogen atom, R^{5a} represents a hydrogen atom, R^{5b} represents a hydrogen atom, a bromine atom, a chlorine atom, a fluorine atom or a methyl group, R^{5c} represents a hydrogen atom, and R^{5d} represents a hydrogen atom.

In another preferred embodiment, the invention relates to compounds of formula (I), supra, wherein R² represents a methyl- group, R^{4b} represents a hydrogen atom, R^{5a} represents a hydrogen atom, R^{5b} represents a hydrogen atom, a bromine atom, a chlorine atom, a fluorine atom or a methyl group, R^{5c} represents a hydrogen atom, R^{5d} represents a hydrogen atom, and R⁶ represents a hydrogen atom.

In another preferred embodiment, the invention relates to compounds of formula (I), supra, wherein R⁶ represents a hydrogen atom or group selected from: C₁-C₃-alkyl-, C₁-C₃-alkoxy-(L²)-, hydroxy-C₁-C₃-alkyl-, aryl-(L²)-, heteroaryl-(L²)-, and wherein L² represents —CH₂— or —CH₂CH₂—.

In another preferred embodiment, the invention relates to compounds of formula (I), supra, wherein R⁶ represents a hydrogen atom or group selected from: C₁-C₃-alkyl-, C₁-C₃-alkoxy-(L²)-, hydroxy-C₁-C₃-alkyl.

In another preferred embodiment, the invention relates to compounds of formula (I), supra, wherein R⁶ represents a hydrogen atom or group selected from: C₁-C₃-alkyl-, C₁-C₃-alkoxy-(L²)-, hydroxy-C₁-C₃-alkyl, and wherein L² represents —CH₂— or —CH₂CH₂—.

In another preferred embodiment, the invention relates to compounds of formula (I), supra, wherein R⁶ represents a hydrogen atom or group selected from: aryl-(L²)-, heteroaryl-(L²)-.

In another preferred embodiment, the invention relates to compounds of formula (I), supra, wherein R⁶ represents a hydrogen atom or group selected from: aryl-(L²)-, heteroaryl-(L²)-, and wherein L² represents —CH₂— or —CH₂CH₂—.

In another preferred embodiment, the invention relates to compounds of formula (I), supra, wherein R⁶ represents a hydrogen atom.

In another preferred embodiment, the invention relates to compounds of formula (I), supra, wherein R² represents a methyl- group, and R⁶ represents a hydrogen atom.

In another preferred embodiment, the invention relates to compounds of formula (I), supra, wherein R⁷ represents a group selected from: oxo, C₁-C₃-alkyl-, fluoro-C₁-C₃-alkyl-, C₁-C₃-alkoxy-, fluoro-C₁-C₃-alkoxy-, —OH, —CN, halo-, —C(═O)R⁸, —C(═O)—O—R⁸, —C(═O)N(R^{8a})R^{8b}, —S(═O)₂R⁸, phenyl-.

In another preferred embodiment, the invention relates to compounds of formula (I), supra, wherein R⁷ represents a group selected from: C₁-C₃-alkyl-, C₁-C₃-alkoxy-, fluoro-C₁-C₃-alkoxy-, —OH, —CN, halo-, —S(═O)₂R⁸.

In another preferred embodiment, the invention relates to compounds of formula (I), supra, wherein R⁷ represents a group selected from: C₁-C₃-alkoxy-, fluoro-C₁-C₃-alkoxy-, —OH, —CN, halo-, —S(═O)₂R⁸.

In another preferred embodiment, the invention relates to compounds of formula (I), supra, wherein R⁷ represents a group selected from: C₁-C₃-alkyl-, C₁-C₃-alkoxy-, —CN, halo-, —C(═O)N(R^{8a})R^{8b}, —S(═O)₂R⁸.

In another preferred embodiment, the invention relates to compounds of formula (I), supra, wherein R⁷ represents a group selected from: C₁-C₃-alkoxy-, —CN, halo-, —S(═O)₂R⁸.

In another preferred embodiment, the invention relates to compounds of formula (I), supra, R⁷ represents a group selected from: methyl-, ethyl-, methoxy-, —CN, fluoro-, —C(═O)N(H)CH₃, —S(═O)₂CH₂—CH₃.

In another preferred embodiment, the invention relates to compounds of formula (I), supra, wherein R⁷ represents a group selected from: methoxy-, —CN, —F, —S(═O)₂—CH₃.

In another preferred embodiment, the invention relates to compounds of formula (I), supra, wherein R⁷ represents a group selected from: —CN, —F.

In another preferred embodiment, the invention relates to compounds of formula (I), supra, wherein R⁷ represents a —CN group.

In another preferred embodiment, the invention relates to compounds of formula (I), supra, wherein R⁷ represents a —F group.

In another preferred embodiment, the invention relates to compounds of formula (I), supra, wherein R⁷ represents a —C(═O)N(R^{8a})R^{8b} group.

In another preferred embodiment, the invention relates to compounds of formula (I), supra, wherein R⁷ represents a —C(═O)N(H)CH₃ group.

In another preferred embodiment, the invention relates to compounds of formula (I), supra, wherein R⁷ represents a C₁-C₃-alkyl- group.

In another preferred embodiment, the invention relates to compounds of formula (I), supra, wherein R⁷ represents a methyl- or ethyl- group.

In another preferred embodiment, the invention relates to compounds of formula (I), supra, wherein R⁷ represents a methyl- group.

In another preferred embodiment, the invention relates to compounds of formula (I), supra, wherein R⁸ represents a hydrogen atom or a C₁-C₆-alkyl- or benzyl- group.

In another preferred embodiment, the invention relates to compounds of formula (I), supra, wherein R⁸ represents a hydrogen atom or a C₁-C₆-alkyl-group.

In another preferred embodiment, the invention relates to compounds of formula (I), supra, wherein R⁸ represents a C₁-C₃-alkyl- group.

In another preferred embodiment, the invention relates to compounds of formula (I), supra, wherein R⁸ represents a hydrogen atom or a methyl- group.

In another preferred embodiment, the invention relates to compounds of formula (I), supra, wherein R⁸ represents a methyl or ethyl group.

In another preferred embodiment, the invention relates to compounds of formula (I), supra, wherein R⁸ represents a methyl- group.

In another preferred embodiment, the invention relates to compounds of formula (I), supra, wherein R^{8a}, R^{8b} represent, independently from each other, a hydrogen atom, or a C₁-C₁₀-alkyl-, C₃-C₇-cycloalkyl-, (C₃-C₇-cycloalkyl)-(L³)-, 4- to 10-membered heterocycloalkyl-,

(4- to 10-membered heterocycloalkyl)-(L³)-, phenyl-, heteroaryl-, phenyl-(L³)-, (phenyl)-O-(L³)-, heteroaryl-(L³)-, or
(aryl)-(4- to 10-membered heterocycloalkyl)- group;
said C₁-C₁₀-alkyl-, C₃-C₇-cycloalkyl-, (C₃-C₇-cycloalkyl)-(L³)-, 4- to 10-membered heterocycloalkyl-,
(4- to 10-membered heterocycloalkyl)-(L³)-, phenyl-, heteroaryl-, phenyl-(L³)-, (phenyl)-O-(L³)-, heteroaryl-(L³)-, and
(aryl)-(4- to 10-membered heterocycloalkyl)- group being optionally substituted one or more times, identically or differently, with R⁹.

In another preferred embodiment, the invention relates to compounds of formula (I), supra, wherein R^{8a}, R^{8b} represent, independently from each other, a hydrogen atom, or a C₁-C₁₀-alkyl- group; said C₁-C₁₀-alkyl- group being optionally substituted one or more times, identically or differently, with R⁹.

In another preferred embodiment, the invention relates to compounds of formula (I), supra, wherein R^{8a} and R^{8b}, together with the nitrogen atom they are attached to, represent a 4- to 10-membered heterocycloalkyl-group, said 4- to 10-membered heterocycloalkyl-group being optionally substituted one or more times, identically or differently, with R⁹.

In another preferred embodiment, the invention relates to compounds of formula (I), supra, wherein R^{8a} and R^{8b}, together with the nitrogen atom they are attached to, represent a 4- to 10-membered heterocycloalkyl-group, said 4- to 10-membered heterocycloalkyl-group being optionally substituted one or more times, identically or differently, with R⁹.

In another preferred embodiment, the invention relates to compounds of formula (I), supra, wherein R⁹ represents a halogen atom, or a oxo, C₁-C₃-alkyl-, halo-C₁-C₃-alkyl-, hydroxy-C₁-C₃-alkyl-, —CN, —C(═O)R¹⁰, —C(═O)N(H)R¹⁰, —C(═O)N(R^{10a})R^{10b}, —C(═O)O—R¹⁰, —N(R^{10a})R^{10b}, —NO₂, —N(H)C(═O)R¹⁰, —N(R^{10a})C(═O)R^{10b}, —N(H)C(═O)N(R^{10a})R^{10b}, —N(R^{10a})C(═O)N(R^{10b})R^{10c}, —N(H)S(═O)₂R¹⁰, —N(R^{10a})S(═O)₂R^{10b}, —OR¹⁰, —O(C═O)R¹⁰, —O(C═O)OR¹⁰ or a tetrazolyl- group.

In another preferred embodiment, the invention relates to compounds of formula (I), supra, wherein R⁹ represents a halogen atom, or a oxo, C₁-C₃-alkyl-halo-C₁-C₃-alkyl-, hydroxy-C₁-C₃-alkyl-, —CN, —C(═O)R¹⁰, —C(═O)N(H)R¹⁰, —C(═O)N(R^{10a})R^{10b}, —C(═O)O—R¹⁰, —N(R^{10a})R^{10b}, —N(R^{10a})C(═O)R^{10b}, —N(R^{10a})C(═O)N(R^{10b})R^{10c}, —N(R^{10a})S(═O)₂R^{10b}, —OR¹⁰, or a tetrazolyl- group.

In another preferred embodiment, the invention relates to compounds of formula (I), supra, wherein R⁹ represents a halogen atom, or a C₁-C₃-alkyl-, hydroxy-C₁-C₃-alkyl-, —CN, —C(═O)N(H)R¹⁰, —C(═O)N(R^{10a})R^{10b}, —C(═O)O—R¹⁰, —N(R^{10a})R^{10b}, —N(R^{10a})C(═O)R^{10b}, —N(R^{10a})C(═O)N(R^{10b})R^{10c}, —OR¹⁰, or a tetrazolyl-group.

In another preferred embodiment, the invention relates to compounds of formula (I), supra, wherein R¹⁰, R^{10a}, R^{10b}, R^{10c} represent, independently from each other, a hydrogen atom or a group selected from: methyl-, hydroxy-ethyl-, methoxy-ethyl-.

In another preferred embodiment, the invention relates to compounds of formula (I), supra, wherein R¹⁰ represents a hydrogen atom or a methyl- group.

In another preferred embodiment, the invention relates to compounds of formula (I), supra, wherein R^{10a} represents a hydrogen atom or a methyl-group.

In another preferred embodiment, the invention relates to compounds of formula (I), supra, wherein R^{10b} represents a hydrogen atom or a group selected from: methyl-, hydroxy-ethyl-, methoxy-ethyl-.

In another preferred embodiment, the invention relates to compounds of formula (I), supra, wherein R^{10b} represents a hydrogen atom or a C₁-C₃-alkyl-group.

In another preferred embodiment, the invention relates to compounds of formula (I), supra, wherein R^{10b} represents a hydrogen atom or a methyl- or ethyl- group.

In another preferred embodiment, the invention relates to compounds of formula (I), supra, wherein R¹¹ represents a hydrogen atom or a cyano-, —C(═O)R¹⁰, or —C(═O)O—R¹⁰ group.

In another preferred embodiment, the invention relates to compounds of formula (I), supra, wherein R¹¹ represents a hydrogen atom or a cyano- or —C(═O)O—R¹⁰ group.

In another preferred embodiment, the invention relates to compounds of formula (I), supra, wherein R¹¹ represents a —C(═O)O—R¹⁰ group.

In another preferred embodiment, the invention relates to compounds of formula (I), supra, wherein R¹¹ represents a cyano- group.

In another preferred embodiment, the invention relates to compounds of formula (I), supra, wherein R¹¹ represents a hydrogen atom.

In another preferred embodiment, the invention relates to compounds of formula (I), supra, wherein L¹ represents a group selected from: —C₁-C₄-alkylene-, —CH₂—CH₂—O—.

In another preferred embodiment, the invention relates to compounds of formula (I), supra, wherein L¹ represents a —C₁-C₄-alkylene- group.

In another preferred embodiment, the invention relates to compounds of formula (I), supra, wherein L¹ represents a —C₁-C₃-alkylene- group.

In another preferred embodiment, the invention relates to compounds of formula (I), supra, wherein L¹ represents a group selected from: —CH₂—, —CH₂—CH₂—, —C(H)(CH₃)—.

In another preferred embodiment, the invention relates to compounds of formula (I), supra, wherein L¹ represents a group selected from: —CH₂—, —C(H)(CH₃)—.

In another preferred embodiment, the invention relates to compounds of formula (I), supra, wherein L¹ represents a —CH₂— group.

In another preferred embodiment, the invention relates to compounds of formula (I), supra, wherein L¹ represents a —CH₂— group, and in which compounds R² represents a methyl- group, R^{4b} represents a hydrogen atom and R⁶ represents a hydrogen atom.

In another preferred embodiment, the invention relates to compounds of formula (I), supra, wherein L² represents a group selected from: —CH₂—, —CH₂—CH₂—.

In another preferred embodiment, the invention relates to compounds of formula (I), supra, wherein L² represents a —CH₂— group.

In another preferred embodiment, the invention relates to compounds of formula (I), supra, wherein L³ represents a —CH₂— group.

In another preferred embodiment, the invention relates to compounds of formula (I), supra, wherein p is an integer of 0.

In another preferred embodiment, the invention relates to compounds of formula (I), supra, wherein p is an integer of 0, and in which compounds R² represents a methyl- or trifluoromethyl- group, R^{4b} represents a hydrogen atom and R⁶ represents a hydrogen atom.

It is to be understood that the present invention relates to any sub-combination within any embodiment of compounds of general formula (I), supra.

Some further examples of combinations are given hereinafter. However, the invention is not limited to these combinations.

In a preferred embodiment, the present invention relates to compounds of general formula (I):

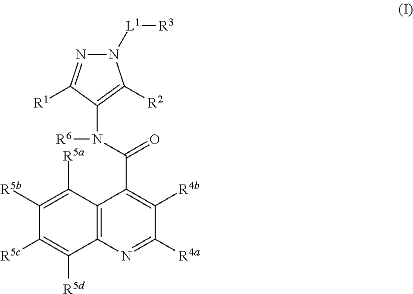

in which:
R¹ represents a hydrogen atom;
R² represents a C₁-C₃-alkyl-, —C(═O)O—R¹⁰ or —C(═O)N(R^{10a})R^{10b} group;
R³ represents a group selected from: phenyl-, heteroaryl-, C₅-C₆-cycloalkyl-, and 5- to 6-membered heterocycloalkyl-;
   wherein said phenyl-, heteroaryl-, C₅-C₆-cycloalkyl-, and 5- to 6-membered heterocycloalkyl- group is optionally substituted, one or more times, identically or differently, with —R⁷;
R^{4a} represents a hydrogen atom or a halogen atom or a group selected from: cyano-, hydroxy-, C₁-C₃-alkyl-, halo-C₁-C₃-alkyl-, C₁-C₃-alkoxy-, C₃-C₇-cycloalkyl-, —C(═O)N(R^{10a})R^{10b};
R^{4b} represents a hydrogen;
R^{5a}, R^{5b}, R^{5c}, R^{5d}
   independently from each other represent a hydrogen atom, a halogen atom or a C₁-C₃-alkyl- group;
R⁶ represents a hydrogen atom;
R⁷ represents a group selected from: C₁-C₃-alkyl-, C₁-C₃-alkoxy-, —CN, halo-, —C(═O)N(R^{8a})R^{8b}, —S(═O)₂R⁸;
R⁸ represents a hydrogen atom or a C₁-C₃-alkyl- group;
R^{8a}, R^{8b}
   represent, independently from each other, a hydrogen atom, or a C₁-C₃-alkyl- group;
R¹⁰, R^{10a}, R^{10b}
   represent, independently from each other, a hydrogen atom or a C₁-C₃-alkyl- group;
   L¹ represents a —C₁-C₄-alkylene- group;
      or a tautomer, a stereoisomer, an N-oxide, a hydrate, a solvate, or a salt thereof, or a mixture of same.

In another preferred embodiment, the present invention relates to compounds of general formula (I):

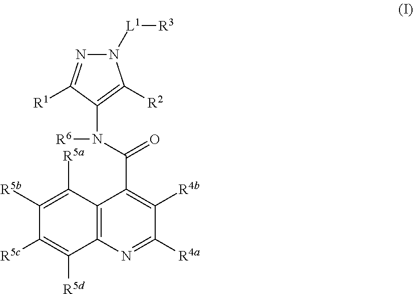

in which:
R¹ represents a hydrogen atom;
R² represents a C₁-C₃-alkyl-, —C(═O)O—R¹⁰ or —C(═O)N(R^{10a})R^{10b} group;
R³ represents a group selected from: phenyl-, heteroaryl-, C₅-C₆-cycloalkyl-, and 5- to 6-membered heterocycloalkyl-;
   wherein said phenyl-, heteroaryl-, C₅-C₆-cycloalkyl-, and 5- to 6-membered heterocycloalkyl- group is optionally substituted, one or two times, identically or differently, with —R⁷;
R^{4a} represents a hydrogen atom or a halogen atom or a group selected from: C₁-C₃-alkyl-, halo-C₁-C₃-alkyl-, C₁-C₃-alkoxy-, C₃-C₇-cycloalkyl-, —C(═O)N(R^{10a})R^{10b};
R^{4b} represents a hydrogen;
R^{5a}, R^{5b}, R^{5c}, R^{5d}
   independently from each other represent a hydrogen atom, a halogen atom or a C₁-C₃-alkyl- group;
R⁶ represents a hydrogen atom;
R⁷ represents a group selected from: C₁-C₃-alkyl-, C₁-C₃-alkoxy-, —CN, halo-, —C(═O)N(R^{8a})R^{8b}, —S(═O)₂R⁸;
R⁸ represents a hydrogen atom, an ethyl- or a methyl- group;
R^{8a}, R^{8b}
   represent, independently from each other, a hydrogen atom, or an ethyl- or a methyl- group;
R¹⁰, R^{10a}, R^{10b}
   represent, independently from each other, a hydrogen atom or an ethyl- or a methyl- group;
L¹ represents a —CH₂— group;
   or a tautomer, a stereoisomer, an N-oxide, a hydrate, a solvate, or a salt thereof, or a mixture of same.

In another preferred embodiment, the present invention relates to compounds of general formula (I):

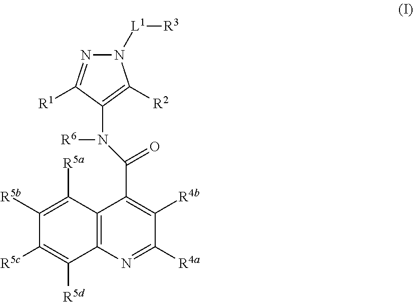

in which:
R¹ represents a hydrogen atom;
R² represents a group selected from: methyl-, —C(═O)NH₂, —C(═O)N(H)CH₃, —C(═O)O—CH₃, —C(═O)OH;
R³ represents a phenyl- group; wherein said phenyl- group is optionally substituted, one or two times, with fluoro; or
R³ represents a phenyl- group; wherein said phenyl- group is optionally substituted, one time, with cyano; or
R³ represents a phenyl- group; wherein said phenyl- group is optionally substituted, one time, with methoxy; or
R³ represents a pyrazolyl- group; wherein said group is optionally substituted with a methyl group; or
R³ represents an oxazolyl- group; wherein said group is optionally substituted with a methyl group; or
R³ represents a thiazolyl- group; wherein said group is optionally substituted with a methyl group; or
R³ represents an oxadiazolyl- group; wherein said group is optionally substituted with a —C(═O)N(H)CH₃ group; or
R³ represents a pyridyl- group; or
R³ represents a cyclohexyl- group; or
R³ represents a piperidinyl- group; wherein said group is optionally substituted with a —S(═O)₂—CH₂—CH₃ group;
R^{4a} represents a —C(═O)NH₂ group; or
R^{4a} represents a —CF₃ group; or
R^{4a} represents a methoxy group; or
R^{4a} represents a methyl group; or
R^{4a} represents a cyclopropyl group;
R^{4b} represents a hydrogen;
R^{5a} represents a hydrogen atom;
R^{5b} represents a hydrogen atom; or
R^{5b} represents a bromine atom or a chlorine atom or a fluorine atom; or
R^{5b} represents a methyl group;
R^{5c} represents a hydrogen atom; or
R^{5c} represents a fluorine atom;
R^{5d} represents a hydrogen atom; or
R^{5d} represents a chlorine atom;
R⁶ represents a hydrogen atom;
L¹ represents a —CH₂— group;
   or a tautomer, a stereoisomer, an N-oxide, a hydrate, a solvate, or a salt thereof, or a mixture of same.

In another preferred embodiment, the present invention relates to compounds of general formula (I):

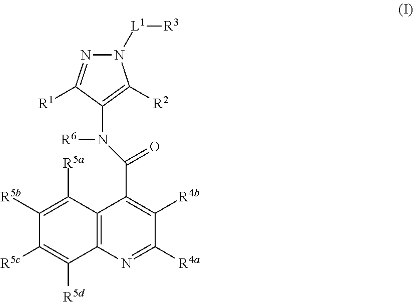

in which:
R¹ represents a hydrogen atom;
R² represents a group selected from: methyl-, —C(═O)NH₂, —C(═O)N(H)CH₃, —C(═O)O—CH₃, —C(═O)OH;
R³ represents a phenyl- group; wherein said phenyl- group is substituted, one or two times, with fluoro; or
R³ represents a phenyl- group; wherein said phenyl- group is substituted, one time, with cyano; or
R³ represents a phenyl- group; wherein said phenyl- group is substituted, one time, with methoxy; or
R³ represents a pyrazolyl- group; wherein said group is substituted, one time, with a methyl group; or
R³ represents an isoxazolyl- group; wherein said group is substituted, one time, with a methyl group; or
R³ represents a thiazolyl- group; wherein said group is substituted, one time, with a methyl group; or
R³ represents an oxadiazolyl- group; wherein said group is optionally substituted, one time, with a group selected from ethyl-, —C(═O)N(H)CH₃; or
R³ represents a pyridyl- group; or
R³ represents a cyclohexyl- group; or
R³ represents a piperidinyl- group; wherein said group is substituted, one time, with a —S(═O)₂—CH₂—CH₃ group;
R^{4a} represents a —C(═O)NH₂ group; or
R^{4a} represents a —CF₃ group; or
R^{4a} represents a methoxy group; or
R^{4a} represents a methyl group; or
R^{4a} represents a cyclopropyl group;
R^{4b} represents a hydrogen;
R^{5a} represents a hydrogen atom;
R^{5b} represents a hydrogen atom; or
R^{5b} represents a bromine atom or a chlorine atom or a fluorine atom; or
R^{5b} represents a methyl group;
R^{5b} represents a hydrogen atom; or
R^{5c} represents a fluorine atom;
R^{5d} represents a hydrogen atom; or
R^{5d} represents a chlorine atom;
R⁶ represents a hydrogen atom;
L¹ represents a —CH₂— group;
   or a tautomer, a stereoisomer, an N-oxide, a hydrate, a solvate, or a salt thereof, or a mixture of same.

In accordance with another aspect, the present invention covers methods of preparing compounds of the present invention, said methods comprising the steps as described in the Experimental Section herein.

In a preferred embodiment, the present invention relates to a method of preparing compounds of general formula (I), supra, in which method an intermediate compound of general formula (II):

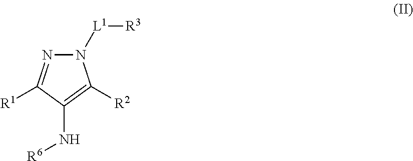

in which R¹, R², R³, R⁶ and L¹ are as defined for the compounds of general formula (I), supra;
is allowed to react with a compound of general formula (III):

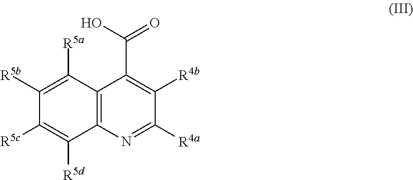

in which R^{4a}, R^{4b}, R^{5a}, R^{5b}, R^{5c}, and R^{5d} are as defined for the compounds of general formula (I), supra;
thus providing a compound of general formula (I):

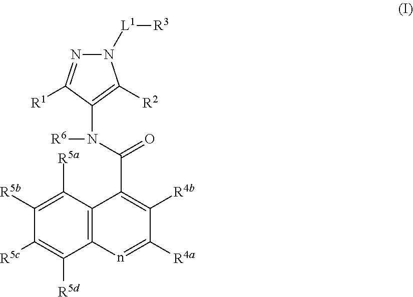

in which R¹, R², R³, R^{4a}, R^{4b}, R^{5a}, R^{5b}, R^{5b}, R^{5d}, R⁶, and L¹ are as defined for the compounds of general formula (I), supra.

In accordance with a further aspect, the present invention covers intermediate compounds which are useful in the preparation of compounds of the present invention of general formula (I), particularly in the method described herein.

In particular, the present invention covers compounds of general formula (II):

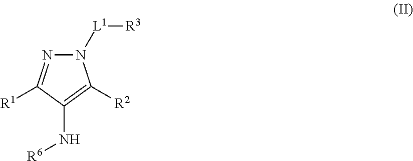

in which R¹, R², R³, R⁶ and L¹ are as defined for the compounds of general formula (I), supra.

In another preferred embodiment, the present invention covers intermediate compounds which are useful in the preparation of compounds of the present invention of general formula (I), particularly in the method described herein. In particular, the present invention covers compounds of general formula (III):

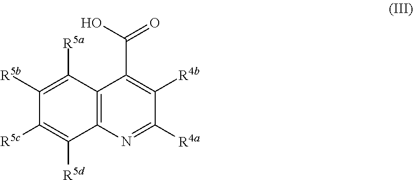

in which R^{4a}, R^{4b}, R^{5a}, R^{5b}, R^{5c}, and R^{5d} are as defined for the compounds of general formula (I), supra.

In accordance with yet another aspect, the present invention covers the use of the intermediate compounds of general formula (II):

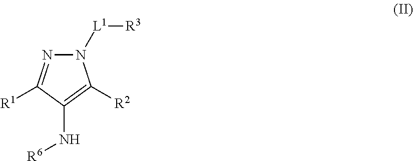

in which R¹, R², R³, R⁶ and L¹ are as defined for the compounds of general formula (I), supra;
for the preparation of a compound of general formula (I) as defined supra.

In another preferred embodiment, the present invention covers the use of the intermediate compounds of general formula (III):

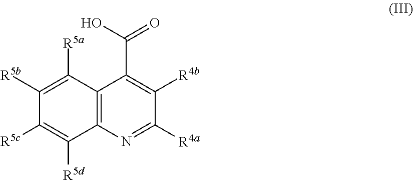

in which R^{4a}, R^{4b}, R^{5a}, R^{5b}, R^{5c}, and R^{5d} are as defined for the compounds of general formula (I), supra;
for the preparation of a compound of general formula (I) as defined supra.

As one of ordinary skill in the art is aware of, the methods described above may comprise further steps like e.g. the introduction of a protective group and the cleavage of the protective group.

This invention also relates to pharmaceutical compositions containing one or more compounds of the present invention. These compositions can be utilised to achieve the desired pharmacological effect by administration to a patient in need thereof. A patient, for the purpose of this invention, is a mammal, including a human, in need of treatment for the particular condition or disease. Therefore, the present invention includes pharmaceutical compositions that are comprised of a pharmaceutically acceptable carrier and a pharmaceutically effective amount of a compound, or salt thereof, of the present invention. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. A pharmaceutically effective amount of compound is preferably that amount which produces a result or exerts an influence on the particular condition being treated. The compounds of the present invention can be administered with pharmaceutically-acceptable carriers well known in the art using any effective conventional dosage unit forms, including immediate, slow and timed release preparations, orally, parenterally, topically, nasally, ophthalmically, optically, sublingually, rectally, vaginally, and the like.

The compounds of this invention can be administered as the sole pharmaceutical agent or in combination with one or more other pharmaceutical agents where the combination causes no unacceptable adverse effects. The present invention relates also to such combinations. For example, the compounds of this invention can be combined with known anti-hyper-proliferative or other indication agents, and the like, as well as with admixtures and combinations thereof. Other indication agents include, but are not limited to, anti-angiogenic agents, mitotic inhibitors, alkylating agents, anti-metabolites, DNA-intercalating antibiotics, growth factor inhibitors, cell cycle inhibitors, enzyme inhibitors, toposisomerase inhibitors, biological response modifiers, or anti-hormones.

Preferred additional pharmaceutical agents are: 131I-chTNT, abarelix, abiraterone, aclarubicin, aldesleukin, alemtuzumab, alitretinoin, altretamine, aminoglutethimide, amrubicin, amsacrine, anastrozole, arglabin, arsenic trioxide, asparaginase, azacitidine, basiliximab, BAY 80-6946, BAY 1000394, BAY 86-9766 (RDEA 119), belotecan, bendamustine, bevacizumab, bexarotene, bicalutamide, bisantrene, bleomycin, bortezomib, buserelin, busulfan, cabazitaxel, calcium folinate, calcium levofolinate, capecitabine, carboplatin, carmofur, carmustine, catumaxomab, celecoxib, celmoleukin, cetuximab, chlorambucil, chlormadinone, chlormethine, cisplatin, cladribine, clodronic acid, clofarabine, crisantaspase, cyclophosphamide, cyproterone, cytarabine, dacarbazine, dactinomycin, darbepoetin alfa, dasatinib, daunorubicin, decitabine, degarelix, denileukin diftitox, denosumab, deslorelin, dibrospidium chloride, docetaxel, doxifluridine, doxorubicin, doxorubicin+estrone, eculizumab, edrecolomab, elliptinium acetate, eltrombopag, endostatin, enocitabine, epirubicin, epitiostanol, epoetin alfa, epoetin beta, eptaplatin, eribulin, erlotinib, estradiol, estramustine, etoposide, everolimus, exemestane, fadrozole, filgrastim, fludarabine, fluorouracil, flutamide, formestane, fotemustine, fulvestrant, gallium nitrate, ganirelix, gefitinib, gemcitabine, gemtuzumab, glutoxim, goserelin, histamine dihydrochloride, histrelin, hydroxycarbamide, I-125 seeds, ibandronic acid, ibritumomab tiuxetan, idarubicin, ifosfamide, imatinib, imiquimod, improsulfan, interferon alfa, interferon beta, interferon gamma, ipilimumab, irinotecan, ixabepilone, lanreotide, lapatinib, lenalidomide, lenograstim, lentinan, letrozole, Leuprorelin, levamisole, lisuride, lobaplatin, lomustine, lonidamine, masoprocol, medroxyprogesterone, megestrol, melphalan, mepitiostane, mercaptopurine, methotrexate, methoxsalen, Methyl aminolevulinate, methyltestosterone, mifamurtide, miltefosine, miriplatin, mitobronitol, mitoguazone, mitolactol, mitomycin, mitotane, mitoxantrone, nedaplatin, nelarabine, nilotinib, nilutamide, nimotuzumab, nimustine, nitracrine, ofatumumab, omeprazole, oprelvekin, oxaliplatin, p53 gene therapy, paclitaxel, palifermin, palladium-103 seed, pamidronic acid, panitumumab, pazopanib, pegaspargase, PEG-epoetin beta (methoxy PEG-epoetin beta), pegfilgrastim, peginterferon alfa-2b, pemetrexed, pentazocine, pentostatin, peplomycin, perfosfamide, picibanil, pirarubicin, plerixafor, plicamycin, poliglusam, polyestradiol phosphate, polysaccharide-K, porfimer sodium, pralatrexate, prednimustine, procarbazine, quinagolide, raloxifene, raltitrexed, ranimustine, razoxane, regorafenib, risedronic acid, rituximab, romidepsin, romiplostim, sargramostim, sipuleucel-T, sizofiran, sobuzoxane, sodium glycididazole, sorafenib, streptozocin, sunitinib, talaporfin, tamibarotene, tamoxifen, tasonermin, teceleukin, tegafur, tegafur+gimeracil+oteracil, temoporfin, temozolomide, temsirolimus, teniposide, testosterone, tetrofosmin, thalidomide, thiotepa, thymalfasin, tioguanine, tocilizumab, topotecan, toremifene, tositumomab, trabectedin, trastuzumab, treosulfan, tretinoin, trilostane, triptorelin, trofosfamide, tryptophan, ubenimex, valrubicin, vandetanib, vapreotide, vemurafenib, vinblastine, vincristine, vindesine, vinflunine, vinorelbine, vorinostat, vorozole, yttrium-90 glass microspheres, zinostatin, zinostatin stimalamer, zoledronic acid, zorubicin.

Optional anti-hyper-proliferative agents which can be added to the composition include but are not limited to compounds listed on the cancer chemotherapy drug regimens in the 11^{th} Edition of the Merck Index, (1996), which is hereby incorporated by reference, such as asparaginase, bleomycin, carboplatin, carmustine, chlorambucil, cisplatin, colaspase, cyclophosphamide, cytarabine, dacarbazine, dactinomycin, daunorubicin, doxorubicin (adriamycine), epirubicin, etoposide, 5-fluorouracil, hexamethylmelamine, hydroxyurea, ifosfamide, irinotecan, leucovorin, Lomustine, mechlorethamine, 6-mercaptopurine, mesna, methotrexate, mitomycin C, mitoxantrone, prednisolone, prednisone, procarbazine, raloxifen, streptozocin, tamoxifen, thioguanine, topotecan, vinblastine, vincristine, and vindesine.

Other anti-hyper-proliferative agents suitable for use with the composition of the invention include but are not limited to those compounds acknowledged to be used in the treatment of neoplastic diseases in Goodman and Gilman's The Pharmacological Basis of Therapeutics (Ninth Edition), editor Molinoff et al., publ. by McGraw-Hill, pages 1225-1287, (1996), which is hereby incorporated by reference, such as aminoglutethimide, L-asparaginase, azathioprine, 5-azacytidine cladribine, busulfan, diethylstilbestrol, 2′,2′-difluorodeoxycytidine, docetaxel, erythrohydroxynonyl adenine, ethinyl estradiol, 5-fluorodeoxyuridine, 5-fluorodeoxyuridine monophosphate, fludarabine phosphate, fluoxymesterone, flutamide, hydroxyprogesterone caproate, idarubicin, interferon, medroxyprogesterone acetate, megestrol acetate, melphalan, mitotane, paclitaxel, pentostatin, N-phosphonoacetyl-L-aspartate (PALA), plicamycin, semustine, teniposide, testosterone propionate, thiotepa, trimethylmelamine, uridine, and vinorelbine.

Other anti-hyper-proliferative agents suitable for use with the composition of the invention include but are not limited to other anti-cancer agents such as epothilone and its derivatives, irinotecan, raloxifen and topotecan.

The compounds of the invention may also be administered in combination with protein therapeutics. Such protein therapeutics suitable for the treatment of cancer or other angiogenic disorders and for use with the compositions of the invention include, but are not limited to, an interferon (e.g., interferon .alpha., .beta., or .gamma.) supraagonistic monoclonal antibodies, Tuebingen, TRP-1 protein vaccine, Colostrinin, anti-FAP antibody, YH-16, gemtuzumab, infliximab, cetuximab, trastuzumab, denileukin diftitox, rituximab, thymosin alpha 1, bevacizumab, mecasermin, mecasermin rinfabate, oprelvekin, natalizumab, rhMBL, MFE-CP1+ZD-2767-P, ABT-828, ErbB2-specific immunotoxin, SGN-35, MT-103, rinfabate, AS-1402, B43-genistein, L-19 based radioimmunotherapeutics, AC-9301, NY-ESO-1 vaccine, IMC-1C11, CT-322, rhCC10, r(m)CRP, MORAb-009, aviscumine, MDX-1307, Her-2 vaccine, APC-8024, NGR-hTNF, rhH1.3, IGN-311, Endostatin, volociximab, PRO-1762, lexatumumab, SGN-40, pertuzumab, EMD-273063, L19-IL-2 fusion protein, PRX-321, CNTO-328, MDX-214, tigapotide, CAT-3888, Labetuzumab, alpha-particle-emitting radioisotope-llinked lintuzumab, EM-1421, HyperAcute vaccine, tucotuzumab celmoleukin, galiximab, HPV-16-E7, Javelin—prostate cancer, Javelin—melanoma, NY-ESO-1 vaccine, EGF vaccine, CYT-004-MelQbG10, WT1 peptide, oregovomab, ofatumumab, zalutumumab, cintredekin besudotox, WX-G250, Albuferon, aflibercept, denosumab, vaccine, CTP-37, efungumab, or 131I-chTNT-1/B. Monoclonal antibodies useful as the protein therapeutic include, but are not limited to, muromonab-CD3, abciximab, edrecolomab, daclizumab, gentuzumab, alemtuzumab, ibritumomab, cetuximab, bevicizumab, efalizumab, adalimumab, omalizumab, muromomab-CD3, rituximab, daclizumab, trastuzumab, palivizumab, basiliximab, and infliximab.

Generally, the use of cytotoxic and/or cytostatic agents in combination with a compound or composition of the present invention will serve to:
(1) yield better efficacy in reducing the growth of a tumor or even eliminate the tumor as compared to administration of either agent alone,
(2) provide for the administration of lesser amounts of the administered chemotherapeutic agents,
(3) provide for a chemotherapeutic treatment that is well tolerated in the patient with fewer deleterious pharmacological complications than observed with single agent chemotherapies and certain other combined therapies,
(4) provide for treating a broader spectrum of different cancer types in mammals, especially humans,
(5) provide for a higher response rate among treated patients,
(6) provide for a longer survival time among treated patients compared to standard chemotherapy treatments,
(7) provide a longer time for tumor progression, and/or
(8) yield efficacy and tolerability results at least as good as those of the agents used alone, compared to known instances where other cancer agent combinations produce antagonistic effects.

The compounds of formula (I), supra, as described and defined herein have surprisingly been found to effectively and selectively inhibit GLUT1 and may therefore be used for the treatment and/or prophylaxis of diseases of uncontrolled cell growth, proliferation and/or survival, inappropriate cellular immune responses, or inappropriate cellular inflammatory responses, or diseases which are accompanied with uncontrolled cell growth, proliferation and/or survival, inappropriate cellular immune responses, or inappropriate cellular inflammatory responses, such as, for example, haematological tumours, solid tumours, and/or metastases thereof, e.g. Leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.

In accordance with another aspect therefore, the present invention covers a compound of general formula (I), or a stereoisomer, a tautomer, an N-oxide, a hydrate, a solvate, or a salt thereof, particularly a pharmaceutically acceptable salt thereof, or a mixture of same, as described and defined herein, for use in the treatment or prophylaxis of a disease, as mentioned supra.

Another particular aspect of the present invention is the use of a compound of general formula (I), described supra, or a stereoisomer, a tautomer, an N-oxide, a hydrate, a solvate, or a salt thereof, particularly a pharmaceutically acceptable salt thereof, or a mixture of same, for the prophylaxis or treatment of a disease.

Another particular aspect of the present invention is the use of a compound of general formula (I) described supra for manufacturing a pharmaceutical composition for the treatment or prophylaxis of a disease.

The compounds of the present invention can be used in particular in therapy and prevention, i.e. prophylaxis, of tumour growth and metastases, especially in solid tumours of all indications and stages with or without pre-treatment of the tumour growth.

Methods of testing for a particular pharmacological or pharmaceutical property are well known to persons skilled in the art.

The present invention relates to a method for using the compounds of the present invention and compositions thereof, to treat mammalian hyper-proliferative disorders. Compounds can be utilized to inhibit, block, reduce, decrease, etc., cell proliferation and/or cell division, and/or produce apoptosis. This method comprises administering to a mammal in need thereof, including a human, an amount of a compound of this invention, or a pharmaceutically acceptable salt, isomer, polymorph, metabolite, hydrate, solvate or ester thereof; etc. which is effective to treat the disorder. Hyper-proliferative disorders include but are not limited, e.g., psoriasis, keloids, and other hyperplasias affecting the skin, benign prostate hyperplasia (BPH), solid tumors, such as cancers of the breast, respiratory tract, brain, reproductive organs, digestive tract, urinary tract, eye, liver, skin, head and neck, thyroid, parathyroid and their distant metastases. Those disorders also include Lymphomas, sarcomas, and leukemias.

Examples of breast cancer include, but are not limited to invasive ductal carcinoma, invasive lobular carcinoma, ductal carcinoma in situ, and lobular carcinoma in situ.

Examples of cancers of the respiratory tract include, but are not limited to small-cell and non-small-cell lung carcinoma, as well as bronchial adenoma and pleuropulmonary blastoma.

Examples of brain cancers include, but are not limited to brain stem and hypophtalmic glioma, cerebellar and cerebral astrocytoma, medulloblastoma, ependymoma, as well as neuroectodermal and pineal tumor.

Tumors of the male reproductive organs include, but are not limited to prostate and testicular cancer. Tumors of the female reproductive organs include, but are not limited to endometrial, cervical, ovarian, vaginal, and vulvar cancer, as well as sarcoma of the uterus.

Tumors of the digestive tract include, but are not limited to anal, colon, colorectal, esophageal, gallbladder, gastric, pancreatic, rectal, small-intestine, and salivary gland cancers.

Tumors of the urinary tract include, but are not limited to bladder, penile, kidney, renal pelvis, ureter, urethral and human papillary renal cancers.

Eye cancers include, but are not limited to intraocular melanoma and retinoblastoma.

Examples of liver cancers include, but are not limited to hepatocellular carcinoma (liver cell carcinomas with or without fibrolamellar variant), cholangiocarcinoma (intrahepatic bile duct carcinoma), and mixed hepatocellular cholangiocarcinoma.

Skin cancers include, but are not limited to squamous cell carcinoma, Kaposi's sarcoma, malignant melanoma, Merkel cell skin cancer, and non-melanoma skin cancer.

Head-and-neck cancers include, but are not limited to laryngeal, hypopharyngeal, nasopharyngeal, oropharyngeal cancer, lip and oral cavity cancer and squamous cell. Lymphomas include, but are not limited to AIDS-related lymphoma, non-Hodgkin's Lymphoma, cutaneous T-cell Lymphoma, Burkitt Lymphoma, Hodgkin's disease, and lymphoma of the central nervous system.

Sarcomas include, but are not limited to sarcoma of the soft tissue, osteosarcoma, malignant fibrous histiocytoma, lymphosarcoma, and rhabdomyosarcoma.

Leukemias include, but are not limited to acute myeloid leukemia, acute lymphoblastic leukemia, chronic lymphocytic leukemia, chronic myelogenous leukemia, and hairy cell leukemia.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a carcinoma.

Based upon standard laboratory techniques known to evaluate compounds useful for the treatment of hyper-proliferative disorders and angiogenic disorders, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, and preferably from about 0.01 mg/kg to about 20 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

General Synthesis of Compounds of General Formula (I) of the Present Invention

The following paragraphs outline a variety of synthetic approaches suitable to prepare compounds of the general formula (I), and intermediates useful for their synthesis.

In addition to the routes described below, also other routes may be used to synthesise the target compounds, in accordance with common general knowledge of a person skilled in the art of organic synthesis. The order of transformations exemplified in the following schemes is therefore not intended to be limiting, and suitable synthesis steps from various schemes can be combined to form additional synthesis sequences. In addition, interconversion of any of the substituents, in particular R¹, R², R^{4a}, R^{4b}, R^{5a}, R^{5b}, R^{5c}, R^{5d} or R⁶, as well as of the R⁷ group attached to R³ via -(L²)ₚ-, can be achieved before and/or after the exemplified transformations. These modifications can be such as the introduction of protective groups, cleavage of protective groups, reduction or oxidation of functional groups, halogenation, metallation, metal catalysed coupling reactions, exemplified by but not limited to Suzuki, Sonogashira and Ullmann coupling, ester saponifications, amide coupling reactions, and/or substitution or other reactions known to a person skilled in the art. These transformations include those which introduce a functionality allowing for further interconversion of substituents. Appropriate protective groups and their introduction and cleavage are well-known to a person skilled in the art (see for example T. W. Greene and P. G. M. Wuts in Protective Groups in Organic Synthesis, 3^{rd} edition, Wiley 1999).

Specific examples of said interconversions are described in the subsequent paragraphs. Further, it is possible that two or more successive steps may be performed without work-up being performed between said steps, e.g. a “one-pot” reaction, as it is well-known to a person skilled in the art.

Compounds of general formula (I) can be assembled from 4-aminopyrazole derivatives of formula (II), in which R¹, R², R³, R⁶ and L¹ are as defined for the compounds of general formula (I), and quinoline-4-carboxylic acid derivatives of formula (III), in which R^{4a}, R^{4b}, R^{5a}, R^{5b}, R^{5c} and R^{5d} are as defined for the compounds of general formula (I), by means of carboxamide (or peptide) coupling reaction well known to the person skilled in the art, according to Scheme 1. Said coupling reaction can be performed by reaction of compounds of the formulae (II) and (III) in the presence of a suitable coupling reagent, such as HATU (O-(7-azabenzotriazol-1-yl)-N,N,N,N′-tetramethyluronium hexafluorophosphate), TBTU (O-(benzotriazol-1-yl)-N,N,N′,N′-tetramethyluronium tetrafluoroborate), PyBOP (benzotriazol-1-yl-oxytripyrrolidinophosphonium hexafluorophosphate), or EDC (1-(3-dimethylaminopropyl)-3-ethylcarbodiimide hydrochloride) in combination with HOBt (1-hydroxy-1H-benzotriazole hydrate), in the presence of a base such as an aliphatic or aromatic tertiary amine, preferably a tertiary aliphatic amine of the formula N(C₁-C₄-alkyl)₃, in an appropriate solvent.

Preferred herein is the performance of said carboxamide coupling reaction using O-(benzotriazol-1-yl)-N,N,N′,N′-tetramethyluronium tetrafluoroborate (TBTU) as a coupling agent, in the presence of N,N-diisopropylethylamine as a base, and in tetrahydrofuran as a solvent, within a temperature range from 0° C. to 50° C.

Also preferred herein is the performance of said carboxamide coupling reaction using O-(7-azabenzotriazol-1-yl)-N,N,N,N′-tetramethyluronium hexafluorophosphate (HATU) as a coupling agent, in the presence of N,N-diisopropylethylamine as a base, and in dimethylsulfoxide as a solvent, within a temperature range from 0° C. to 50° C.

Also preferred herein is the performance of said carboxamide coupling reaction using benzotriazol-1-yl-oxytripyrrolidinophosphonium hexafluorophosphate (PyBOP) as a coupling agent, in the presence of N,N-diisopropylethylamine as a base, and in tetrahydrofuran as a solvent, within a temperature range from 0° C. to 50° C.

The preparation of amides from 4-aminopyrazole derivatives of formula (II), in which R¹, R², R³, R⁶ and L¹ are as defined for the compounds of general formula (I), and quinoline-4-carboxylic acid derivatives of formula (III), in which R^{4a}, R^{4b}, R^{5a}, R^{5b}, R^{5c} and R^{5d} are as defined for the compounds of general formula (I), can furthermore be accomplished, as well known to the person skilled in the art, by converting carboxylic acids of the formula (III) into the corresponding acyl halides, e.g. by reacting with a halogenating agent such as thionyl chloride, oxalyl chloride, or phosphoroxy chloride, and subsequent aminolysis using said 4-aminopyrazole derivatives of formula (II).

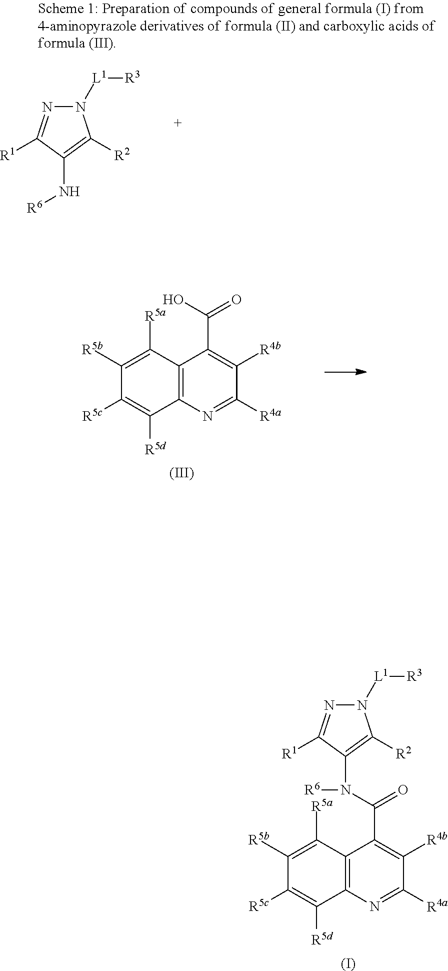

4-Aminopyrazole intermediates and quinazoline-4-carboxylic acid derivatives of formulae (II) and (III) can be prepared using synthetic methods described in more detail as described in Schemes 3a, 3b, 4 and 5 shown below. Certain quinazoline-4-carboxylic acids are also commercially available in some structural variety.

If aminopyrazole derivatives of formula (II), in which R⁶ represents a hydrogen atom, have been employed in the carboxamide coupling reaction described supra, R⁶ groups different from hydrogen can also be introduced subsequently to said carboxamide coupling reaction by means of deprotonating the resulting compounds of formula (Ia), in which R¹, R², R³, R^{4a}, R^{4b}, R^{5a}, R^{5b}, R^{5c}, R^{5d} and L¹ are as defined for the compounds of general formula (I), with a base such as an alkali metal hydride, preferably sodium hydride, followed by reaction with a compound of the formula (IV), in which LG represents a leaving group, preferably chloro, bromo, or iodo, and in which R⁶ is as defined for the compounds of general formula (I) but different from hydrogen, to give compounds of formula (Ib), as outlined in Scheme 2.

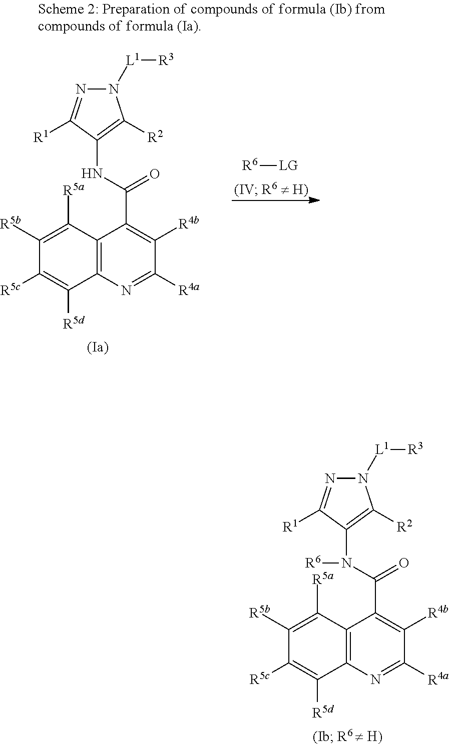

Compounds of formula (IV) are well known to the person skilled in the art and are readily commercially available.

Intermediate 4-aminopyrazole derivatives of formula (II) are available e.g. by reaction of 4-nitropyrazole derivatives of the formula (V), in which R¹ and R² are as defined for the compounds of general formula (I), with compounds of the formula (VI), in which R³ and L¹ are as defined for the compounds of general formula (I), and in which LG represents a leaving group, preferably chloro, bromo, or iodo, in the presence of a suitable base such as an alkali carbonate, e.g. cesium carbonate, or an organic base such as e.g. 1,8-Diazabicyclo[5.4.0]undec-7-ene (DBU) to give N-1-substituted nitropyrazole intermediates of formula (VII) and (XII). Alternatively, the nitro group can be introduced after substitution of pyrazole N-1 with -L¹-R³ described above.

Since R¹ and R² are different from each other, said nitropyrazole intermediates can be formed as mixtures of regioisomers (compounds of formulae (VII) and (XII)), as a result of the tautomery featured by the pyrazole core. Said mixtures can be separated into pure regioisomers by methods known to the person skilled in the art, such as column chromatography on silica gel, or by preparative HPLC, either directly following the reaction, or on a later or final stage.

Said compounds of formula (VII) can subsequently be reduced, using reduction methods well known to the person skilled in the art, to give primary amines of formula (IIa). Said reduction methods encompass the use of palladium catalysed hydrogenation, using elemental hydrogen or alternative hydrogen sources such as ammonium formate, and the use of zinc dust or powdered iron in the presence of acetic acid, or the use of tin (II) chloride e.g. in ethanol as a solvent. The latter reagents are preferably used if the substrate contains functional groups vulnerable to catalytic hydrogenation, such as cyano-, bromo or chloro, in particular if attached to an aromatic ring.

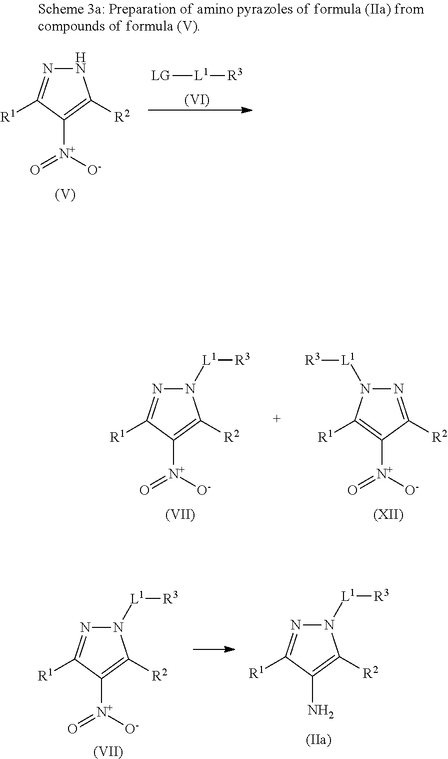

Alternatively, the mixtures of compounds of formula (VII) and (XII) can be reduced to a mixture of the corresponding amines of formulae (IIa) and (IIIb) which are then separated from each other.

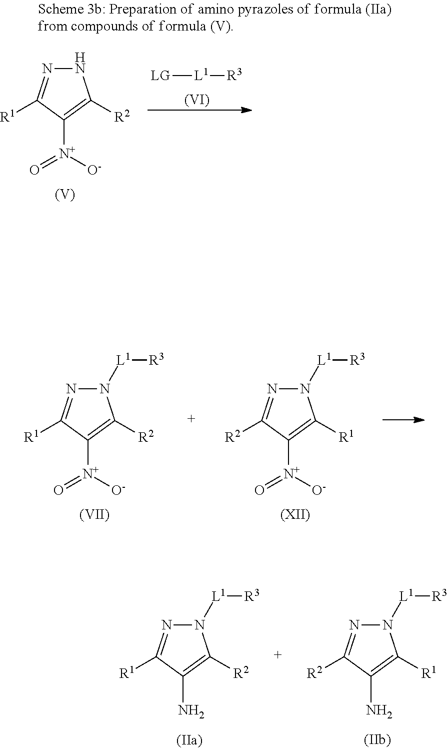

4-Nitropyrazoles of the formula (V) are well known to the person skilled in the art and are readily commercially available, such as e.g. 3-methyl-4-nitro-1H-pyrazole, 4-nitro-1H-pyrazole-3-carbonitrile, methyl 4-nitro-1H-pyrazole-3-carboxylate, 4-nitro-3-(trifluoromethyl)-1H-pyrazole, or can be prepared starting from commercially available and/or known pyrazoles via nitration (e.g. WO2012/62783, Organic Process Research and Development, 2009, p. 698-705).

R⁶ groups different from hydrogen can either be introduced at later stage, as outlined in Scheme 2, or they may be introduced into primary amines by means of reductive amination reactions well known to the person skilled in the art, e.g. by reaction of said primary amines with suitable aldehydes or ketones, followed by reduction e.g. with sodium cyanoborohydride.

Quinoline-4-carboxylic acid derivatives of formula (III), if not commercially available, can be prepared readily from indole-2,3-dione precursors (see e.g. Monatshefte für Chemie 2013, p. 391; Chinese Chemical Letters 2010, p. 35; The Pfitzinger Reaction. (Review) in Chemistry of Heterocyclic Compounds, Vol 40 (2004), Issue 3, pp 257) of formula (VIII), in which R^{5a}, R^{5b}, R^{5c} and R^{5d} are as defined for the compounds of general formula (I), by reaction with carbonyl compounds of formula (IX), in which R^{4a} and R^{4b} are as defined for the compounds of general formula (I), in an aqueous buffered solvent e.g. comprising sodium hydroxide, sodium acetate, acetic acid and water, at an elevated temperature, to directly give compounds of formula (III), as outlined in Scheme 4.

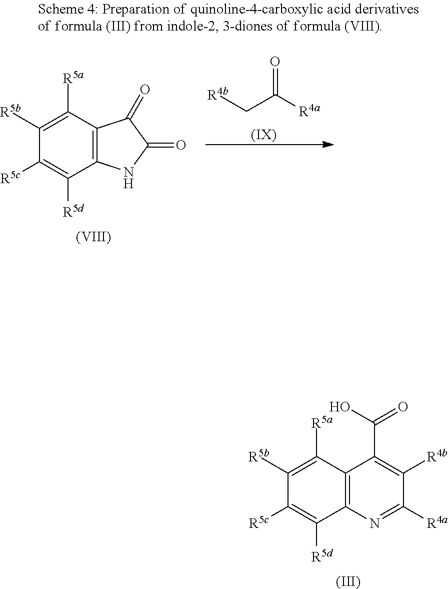

Indole-2,3-diones of formula (VIII) are well known to the person skilled in the art and are either commercially available or can be prepared by methods described e.g. in Chinese Chemical Letters, 2013, p. 929; J. Med. Chem. 2006, p. 4638. Carbonyl compounds of formula (IX) can be purchased commercially in wide structural variety.

The chemical reactivity of groups R^{4a} present in compounds of formula (III) can be modulated as a result of the neighbouring ring nitrogen atom, thus allowing for chemoselective manipulation of R^{4a}. This may be exemplified by (but is not limited to) the synthesis of a subset of said quinoline-4-carboxylic acid derivatives described by formula (IId), in which R^{4a} is represented by a group —C(═O)N(R^{10a})R^{10b}, as outlined in Scheme 5. Diacids of the formula (IIIa), which are available e.g. by reacting pyruvic acid with an indole-2,3-dione of formula (V) according to Scheme 4, can be converted readily into the respective diesters of formula (IIIb), in which R^{4b}, R^{5a}, R^{5b}, R^{5c} and R^{5d} are as defined for the compounds of general formula (I), and in which R^{E} represents C₁-C₃-alkyl-, by conversion of the carboxy groups into acyl halides using methods well known to the person skilled in the art, e.g. by reaction with thionyl chloride, followed by solvolysis in an aliphatic alcohol of the formula C₁-C₃-alkyl-OH, preferably methanol. The resulting diesters of formula (IIIb) are then reacted with an amine of formula (X), in which R^{10a} and R^{10b} are as defined for the compounds of general formula (I), to give monoamides of formula (IIIc), which are subsequently subjected to ester hydrolysis by methods known to the person skilled in the art, preferably by an alkali hydroxide in an aqueous aliphatic alcohol of the formula C₁-C₃-alkyl-OH, to give the quinoline-4-carboxylic acid derivatives of formula (IIId). The sequence of protocols describing the preparation of Intermediate 2A in the experimental part below constitute an instructive example for this reaction sequence.

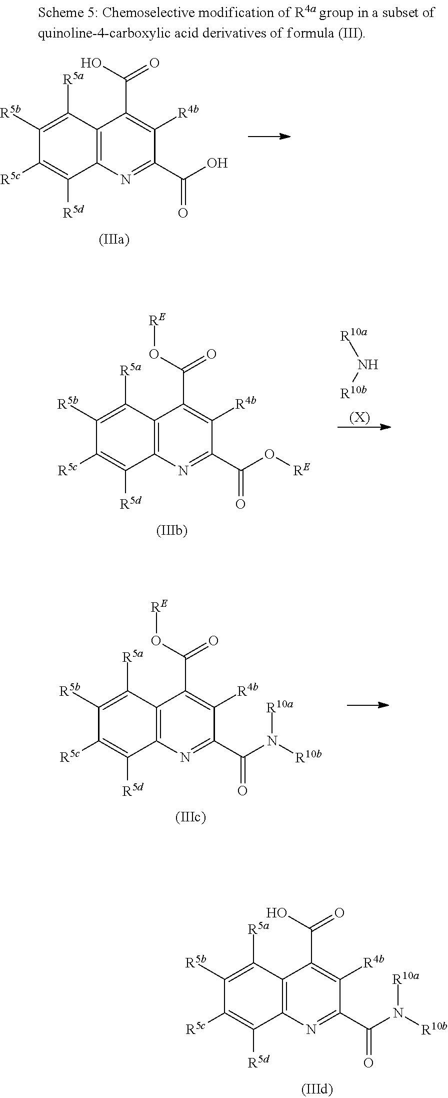

An alternative synthetic approach to the compounds of the general formula (I), which is particularly suitable for the preparation or multiple derivatives featuring different -L¹-R³ moieties by introducing said -L¹-R³ moieties on late stage, is outlined in Scheme 6. 4-Aminopyrazoles of formula (IIc), in which R¹, R² and R⁶ are as defined for the compounds of general formula (I), and quinoline-4-carboxylic acid derivatives of formula (III), in which R^{4a}, R^{4b}, R^{5a}, R^{5b}, R^{5c} and R^{5d} are as defined for the compounds of general formula (I), are subjected to a carboxamide (or peptide) coupling reaction well known to the person skilled in the art, as discussed supra with regard to Scheme 1, to give intermediate compounds of formula (XI). Said coupling reaction can be performed by reaction of compounds of the formulae (IIc) and (III) in the presence of a suitable coupling reagent, such as HATU (O-(7-azabenzotriazol-1-yl)-N,N,N′,N′-tetramethyluronium hexafluorophosphate), TBTU (O-(benzotriazol-1-yl)-N,N,N′,N′-tetramethyluronium tetrafluoroborate), PyBOP (benzotriazol-1-yl-oxytripyrrolidinophosphonium hexafluorophosphate), or EDC (1-(3-dimethylaminopropyl)-3-ethylcarbodiimide hydrochloride) in combination with HOBt (1-hydroxy-1H-benzotriazole hydrate), in the presence of a base such as an aliphatic or aromatic tertiary amine, preferably a tertiary aliphatic amine of the formula N(C₁-C₄-alkyl)₃, in an appropriate solvent.

Participation of the pyrazole ring NH in said carboxamide coupling reaction may give rise to the formation of intermediate compounds of formula (XI) as regioisomeric mixtures with the corresponding N1 amides. These can be removed by separation techniques well known to the person skilled in the art, e.g. preparative HPLC either immediately after the coupling, or, preferably, after conversion into the compounds of general formula (I).

Said intermediate compounds of formula (XI) can be converted into the compounds of general formula (I) by reaction with compounds of the formula (VI), in which R³ and L¹ are as defined for the compounds of general formula (I), and in which LG represents a leaving group, preferably chloro, bromo, or iodo, in the presence of a suitable inorganic base, such as an alkali carbonate, preferably cesium carbonate or an alkali hydride, such as sodium hydride, or an organic base, such as potassium tert.-butoxide or 1,8-diazabicyclo[5.4.0]undec-7-ene.

4-Aminopyrazoles of formula (IIc) are well known to the person skilled in the art and can be purchased commercially in many cases.

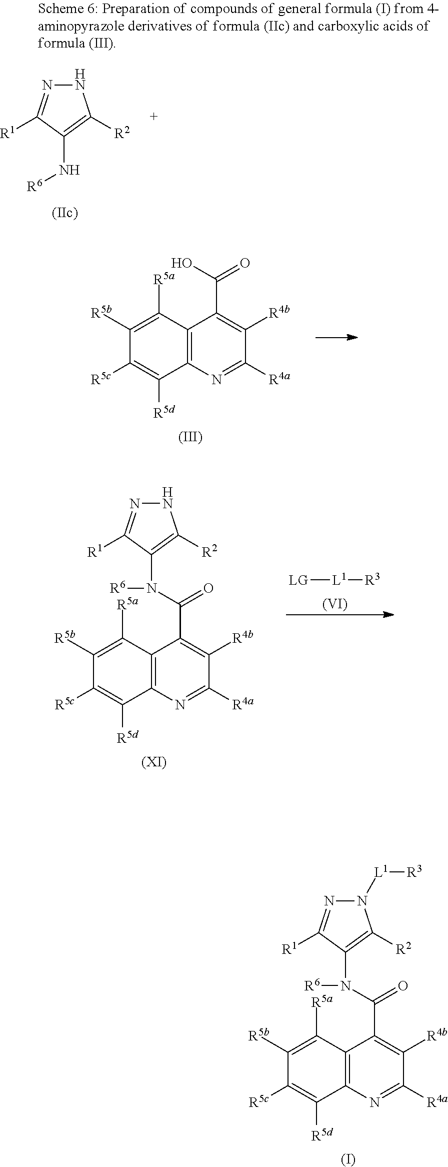

Abbreviations

| | |
|---|---|
| DMF | N,N-dimethylformamide |
| HPLC | high performance liquid chromatography |
| HOBt | 1-hydroxy-1H-benzotriazole hydrate |
| UPLC | ultra performance liquid chromatography |
| DAD | diode array detector |
| EDC | 1-(3-dimethylaminopropyl)-3-ethylcarbodiimide hydrochloride |
| ELSD | evaporative light scattering detector |
| ESI | electrospray ionization |
| DLD1 | colorectal adenocarcinoma cells isolated by D.L. Dexter |
| CHO-K1 | chinese hamster ovary K1 cells |
| H460 | lung carcinoma cells |
| RCC | renal cell carcinoma cells |
| VHL | von Hippel-Lindau |
| DMEM | Dulbecco's modified eagle medium |
| FCS | fetal calf serum |
| HEPES | 4-(2-hydroxyethyl)-1-piperazineethanesulfonic acid |
| HMPA | Hexamethylphosphoramide |
| KRP | Krbes-Ringer phosphate |
| HATU | O-(7-azabenzotriazol-1-yl)-N,N,N′,N′-tetramethyluronium |
| | hexafluorophosphate |
| Xphos | 2-Dicyclohexylphosphino-2′,4′,6′-triisopropylbiphenyl |
| TBTU | O-(Benzotriazol-1-yl)-N,N,N′,N′-tetramethyluronium |
| | tetrafluoroborate |
| PyBOP | Benzotriazol-1-yl-oxytripyrrolidinophosphonium |
| | hexafluorophosphate |
| KP-Sil | ready to use silica gel column |
| DBU | 1,8-Diazabicyclo[5.4.0]undec-7-ene |
| DMSO | Dimethylsulfoxide |

Examples were seperated by the following methods:

Method A: Agilent: Prep 1200, 2×Prep Pump, DLA, MWD, Prep FC; Column: Chiralpak IA 5 μm 250×30 mm; temperature: room temp.; Detection: UV 254 nm.
A1: Solvent: hexane/ethanol/diethylamine 70:30:0.1 (v/v/v); Flow: 50 mL/min
A2: Solvent: hexane/2-propanol 70:30 (v/v); Flow: 50 mL/min
A3: Solvent: hexane/ethanol/diethylamine 70:30:0.1 (v/v/v); Flow: 45 mL/min

Method B: 2× Labomatic Pumpe HD-3000, Labomatic AS-3000, Knauer DAD 2600, Labomatic Labcol Vario 4000 Plus; Column: Xbrigde C18 5 μm 150×50 mm; Solvent: A=water, B=acetonitrile; Flow: 150 mL/min; temperature: room temp.; Detection: UV 280 nm.
B1: Gradient: 0-7 min 50-60% B
B2: Gradient: 0-8 min 54% B
B3: Gradient: 0-1 min 30%, 1-10 min 30-40% B
B4: Gradient: 0-8 min 43% B

Method C: System: Waters autopurification system: Pump 254, Sample Manager 2767, CFO, DAD 2996, SQD 3100; Column: XBridge C18 5 μm 100×30 mm; Solvent: A=H₂O+0.2% Vol. ammonia (32%), B=acetonitrile; Flow: 70 mL/min; temperature: room temp.; Detection: DAD scan range 210-400 nm; MS ESI+, ESI−, scan range 160-1000 m/z.
C1: Gradient: 0.5 min Inlet (21% B, 25 mL/min); 0.5-5.5 min 43-47% B
C2: Gradient: 0-0.5 min 25 mL/min auf 70 mL/min 59% B; 0.5-5.5 min 59% B
C3: Gradient: 0-0.5 min 25 mL/min auf 70 mL/min 52% B; 0.5-5.5 min 52% B
C4: Gradient: 0-0.5 min 25 mL/min auf 70 mL/min 29% B; 0.5-5.5 min 29% B

Method D: System: Waters autopurification system: Pump 254, Sample Manager 2767, CFO, DAD 2996, SQD 3100; Column: YMC C18 5 μm 100×30 mm; Solvent: A=H₂O+0.2% Vol. ammonia (99%), B=acetonitrile; Gradient: 0.5 min Inlet (21% B, 25 mL/min); 0.5-5.5 min 43-47% B; Flow: 70 mL/min; temperature: room temp.; Detection: DAD scan range 210-400 nm; MS ESI+, ESI−, scan range 160-1000 m/z.

Method E: System: Waters autopurification system: Pump 254, Sample Manager 2767, CFO, DAD 2996, SQD 3100; Column: XBridge C18 5 μm 100×30 mm; Solvent: A=H₂O+0.1% Vol. HCOOH (99%), B=acetonitrile; Flow: 70 mL/min; temperature: room temp.; Detection: DAD scan range 210-400 nm; MS ESI+, ESI−, scan range 160-1000 m/z.
E1: Gradient: 0.5 min Inlet (20% B, 25 mL/min); 0.5-5.5 min 20-90% B
E2: Gradient: 0.5 min Inlet (20% B, 25 mL/min); 0.5-5.5 min 20-70% B

Method F: System: Waters autopurification system: Pump 2545, Sample Manager 2767, CFO, DAD 2996, ELSD 2424, SQD; Column: XBridge C18 5 μm 100×30 mm; Solvent: A=H₂O+0.1% Vol. formic acid (99%), B=acetonitrile; Gradient: 0-8 min 10-100% B, 8-10 min 100% B; Flow: 50 mL/min; temperature: room temp.; Solution: Max. 250 mg/max. 2.5 mL DMSO o. DMF; Injection: 1×2.5 mL; Detection: DAD scan range 210-400 nm; MS ESI+, ESI−, scan range 160-1000 m/z.

Method G: System: Waters autopurification system: Pump 2545, Sample Manager 2767, CFO, DAD 2996, ELSD 2424, SQD; Column: XBridge C18 5 μm 100×30 mm; Solvent: A=H₂O+0.1% Vol. ammonia (99%), B=acetonitrile; Gradient: 0-8 min 10-100% B, 8-10 min 100% B; Flow: 50 mL/min; temperature: room temp.; Solution: Max. 250 mg/max. 2.5 mL DMSO o. DMF; Injection: 1×2.5 mL; Detection: DAD scan range 210-400 nm; MS ESI+, ESI−, scan range 160-1000 m/z.

Method H: System: 2× Labomatic Pumpe HD-3000, Labomatic AS-3000, Knauer DAD 2600, Labomatic Labcol Vario 4000 Plus; Column: Chiralpak IB 5 μm 250×30 mm; Flow: 50 mL/min; temperature: room temp.; Solution: Max. 323 mg/3 mL Methylenechloride; Injection: 6×0.5 mL; Detection: UV 254 nm
H1: Gradient: hexane/ethanol/diethylamine 80:20:0.1 (v/v/v)
H2: Gradient: hexane/ethanol/diethylamine 65:35:0.1 (v/v/v)

Method I: System: Sepiatec: Prep SFC100; Column: LUNA HILIC 5 μm 250×30 mm; Solvent: CO₂/methanol 90/10+0.5% NH₃; Flow: 100 mL/min; temperature: 40° C.; Solution: 100 mg in 1.5 mL DMSO; Injection: 5×0.3 mL; Detection: UV 254 nm

Method J: System: Sepiatec: Prep SFC100; Column: Chiralpak IC 5 μm 250×20 mm; Solvent: CO₂/methanol 60/40; Flow: 80 mL/min; temperature: 40° C.; Solution: 229 mg in 3.2 mL methanol; Injection: 16×0.2 mL; Detection: UV 254 nm

Method K: System: Sepiatec: Prep SFC100; Column: Chiralpak IC 5 μm 250×20 mm; Solvent: CO₂/ethanol 87/13; Flow: 80 mL/min; temperature: 40° C.; Solution: 107 mg in 2 mL DMSO; Injection: 5×0.4 mL; Detection: UV 254 nm

Method L: System: Sepiatec: Prep SFC100; Column: Chiralpak IC 5 μm 250×20 mm; Solvent: CO₂/methanol 74/26; Flow: 80 mL/min; temperature: 40° C.; Solution: 55 mg in 1 mL DMSO; Injection: 4×0.25 mL; Detection: UV 254 nm

Method M: System: Sepiatec: Prep SFC100; Column: Chiralpak IC 5 μm 250×20 mm; Solvent: CO₂/methanol 87/13; Flow: 80 mL/min; temperature: 40° C.; Solution: 175 mg in 2 mL DMSO; Injection: 10×0.2 mL; Detection: UV 254 nm

Method N: System: Sepiatec: Prep SFC100; Column: Chiralpak IC 5 μm 250×20 mm; Solvent: CO₂/2-propanol 60/40; Flow: 80 mL/min; temperature: 40° C.; Solution: 66 mg in 1 mL DMSO; Injection: 10×0.1 mL; Detection: UV 254 nm

Method O: System: Sepiatec: Prep SFC100; Column: Chiralpak IE 5 μm 250×20 mm; Solvent: CO₂/2-propanol 88/12; Flow: 80 mL/min; temperature: 40° C.; Solution: 105 mg in 1.8 mL DMSO; Injection: 6×0.3 mL; Detection: UV 254 nm

Method P: System: Sepiatec: Prep SFC100; Column: Chiralpak IC 5 μm 250×20 mm; Solvent: CO₂/2-propanol 70/30; Flow: 80 mL/min; temperature: 40° C.; Solution: 37 mg in 2 mL DMSO; Injection: 4×0.5 mL; Detection: UV 254 nm

Method Q: System: Waters Autopurificationsystem; Column: YMC Triart C18 5 μm 100×30 mm; Solvent: H₂O+0.1 Vol % HCOOH/methanol 99/1; Flow: 70 mL/min; temperature: 22° C.; Solution: 110 mg in 2.5 mL DMSO; Injection: 5×0.5 mL; Detection: DAD scan range 210-400 nm

Method R: System: Agilent: Prep 1200; Column: Chiralpak IE 5 μm 250×20 mm; Solvent: hexane/ethanol 67/33; Flow: 15 mL/min; temperature: 22° C.; Solution: 50 mg in 2 mL DMSO; Injection: 14×0.15 mL; Detection: UV 254 nm

Method S: System: Agilent: Prep 1200; Column: Chiralpak IC 5 μm 250×20 mm; Solvent: hexane/ethanol 79/21; Flow: 15 mL/min; temperature: 22° C.; Solution: 233 mg in 3 mL DMSO; Injection: 30×0.1 mL; Detection: UV 325 nm

Method T: System: Agilent: Prep 1200; Column: Chiralpak IC 5 μm 250×20 mm; Solvent: acetonitrile/ethanol 90/10; Flow: 15 mL/min; temperature: 22° C.; Solution: 211 mg in 2 mL DMSO; Injection: 21×0.1 mL; Detection: UV 254 nm

Method U: System: Sepiatec: Prep SFC100; Column: Chiralpak IE 5 μm 250×20 mm; Solvent: CO₂/ethanol 77/23; Flow: 80 mL/min; temperature: 40° C.; Solution: 210 mg in 2.5 mL DMSO; Injection: 7×0.4 mL; Detection: UV 254 nm

Method V: System: Agilent: Prep 1200; Column: Chiralpak IC 5 μm 250×20 mm; Solvent: acetonitrile+0.1% diethylamine; Flow: 15 mL/min; temperature: 22° C.; Solution: 190 mg in 2.5 mL DMSO; Injection: 20×0.125 mL; Detection: UV 254 nm

Column chromatography was performed on a Biotage® Isolera™ Spektra Four Flash Purification System.

NMR peak forms are stated as they appear in the spectra, possible higher order effects have not been considered. In cases were a signal is very broad or is partially or totally hidden by a solvent peak the total number of hydrogen atoms displayed in NMR spectra can differ from the number of hydrogen atoms present in the respective molecule.

Yields in % reflect the purity of the desired product obtained if not stated otherwise; purities significantly below 90% were specified explicity if appropriate.

If not stated otherwise, starting materials as mentioned in the protocols were purchased from commercial suppliers.

The IUPAC names of the examples and intermediates were generated using the program ‘ACD/Name batch version 12.01’ from ACD LABS, and were adapted if needed.

Intermediates

Intermediate 1A

### 6-bromo-2-(trifluoromethyl)quinoline-4-carboxylic acid

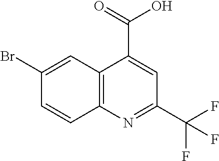 [SMILES: O=C(O)c1cc(C(F)(F)F)nc2ccc(Br)cc12]

300 mg (1.33 mmol) of 5-bromo-1H-indole-2,3-dione was suspended in 3 mL water in a microwave vial. 82 mg (1.46 mmol) potassium hydroxide, 152 μL (2.65 mmol) acetic acid and 152 mg (1.86 mmol) sodium acetate were added so that the pH was around 5. The solution was cooled to 10° C. and 238 μL (2.65 mmol) 1,1,1-trifluoroacetone was added rapidly, the microwave vial was sealed and heated in the microwave for 2 h at 120° C. The reaction was stopped by the addition of 10% aqueous hydrochloric acid solution and the resulting precipitate was isolated by filtration, washed with water and dried in a vacuum drying cabinet at 50° C. overnight to obtain 409 mg (1.28 mmol, 96%) of the desired title compound.

1H NMR (300 MHz, DMSO d₆): δ (ppm)=8.14 (dd, 1H), 8.21 (d, 1H), 8.32 (s, 1H), 9.09 (d, 1H), 14.50 (br. s., 1H).

Intermediate 2A

### 2-carbamoyl-7-fluoroquinoline-4-carboxylic acid

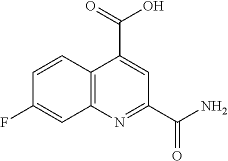 [SMILES: NC(=O)c1cc(C(=O)O)c2ccc(F)cc2n1]

### Step 1: 7-fluoroquinoline-2,4-dicarboxylic acid

To a mixture of 5.0 g (30.3 mmol) 6-fluoro-1H-indole-2,3-dione in 75 mL of 33% aq. potassium hydroxide solution was added 4.67 g (53.0 mmol) pyruvic acid and this mixture was heated at 40° C. for 18 hours. After cooling to room temperature 10% aq. sulfuric acid was added (pH about 1). The formed solid was isolated by filtration and dried in vacuum. The solid was the desired 7-fluoroquinoline-2,4-dicarboxylic acid, which was used without further purification. Yield: 6.02 g (85%)

¹H-NMR (300 MHz, DMSO d₆) δ (ppm)=7.78 (ddd, 1H), 7.99 (dd, 1H), 8.42 (s, 1H), 8.89 (dd, 1H).

### Step 2: dimethyl 7-fluoroquinoline-2,4-dicarboxylate

A mixture of 6.0 g (25.5 mmol) of the diacid of step 1) intermediate 2A) and 28 mL (383 mmol) thionyl chloride was heated at 80° C. for 2 days. After cooling to 25° C. the resulting suspension was evaporated to dryness in vacuum. This crude product was suspended in 47 mL methanol and refluxed for 3 hours. After cooling to 25° C. the formed solid was isolated by filtration. Yield: 3.06 g (44%)

¹H-NMR (300 MHz, DMSO d₆) δ (ppm)=3.98 (s, 3H), 4.01 (s, 3H), 7.85 (ddd, 1H), 8.07 (dd, 1H), 8.45 (s, 1H), 8.80 (dd, 1H).

### Step 3: methyl 2-carbamoyl-7-fluoroquinoline-4-carboxylate

To a solution of 3.05 g (11.6 mmol) diester of step 2) intermediate 2A) in 42 mL methanol was added 41 mL of a 7M solution of ammonia in methanol and stirred for 3.5 hour at 50° C. After cooling to 25° C. the formed solid was isolated by filtration and dried. Using this methodology we obtained the desired methyl 2-carbamoyl-7-fluoroquinoline-4-carboxylate. Yield: 2.33 g (81%)

¹H-NMR (400 MHz, DMSO d₆) δ (ppm)=4.03 (s, 3H), 7.83 (ddd, 1H), 7.94 (dd, 1H), 7.97 (s, 1H), 8.39 (s, 1H), 8.52 (s, 1H), 8.83 (dd, 1H).

### Step 4: 2-carbamoyl-7-fluoroquinoline-4-carboxylic acid

To a solution of 3.00 g (12.1 mmol) of the compound from step 3) intermediate 2A) in 56 mL methanol and 20 ml tetrahydrofuran was added a solution of 4.35 g sodium hydroxide in 111 mL water. This mixture was stirred for 1 hour at 25° C. and then concentrated in vacuum. The residue was diluted with water and 10% aq. sulfuric acid was added up to pH 5. After stirring for additional 15 minutes the formed solid was isolated by filtration and dried in vacuum. Using this methodology we obtained the desired title compound. Yield: 2.38 g (80%)

¹H-NMR (300 MHz, DMSO d₆) δ (ppm)=7.76 (ddd, 1H), 7.84-7.96 (m, 2H), 8.35 (br. s., 1H), 8.46 (s, 1H), 8.89 (dd, 1H), 14.02 (br. s., 1H).

Intermediate 3A

### 2-carbamoylquinoline-4-carboxylic acid

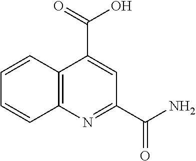 [SMILES: NC(=O)c1cc(C(=O)O)c2ccccc2n1]

### Step 1: dimethyl quinoline-2,4-dicarboxylate

In analogy to step 2) of intermediate 2A), 11.4 g (44.9 mmol) commercially available quinoline-2,4-dicarboxylic acid were reacted to give 6.44 g (59%) dimethyl quinoline-2,4-dicarboxylate.

¹H-NMR (300 MHz, DMSO d₆) δ (ppm)=3.98 (s, 3H), 4.01 (s, 3H), 7.88 (ddd, 1H), 7.96 (ddd, 1H), 8.26 (dd, 1H), 8.46 (s, 1H), 8.70 (dd, 1H).

### Step 2: methyl 2-carbamoylquinoline-4-carboxylate

In analogy to step 3) of intermediate 2A), 1.0 g (4.08 mmol) dimethyl quinoline-2,4-dicarboxylate of step 1) of intermediate 3A) were reacted to give 650 mg (66%) methyl 2-carbamoylquinoline-4-carboxylate.

¹H-NMR (400 MHz, DMSO d₆) δ (ppm)=4.01 (s, 3H), 7.85 (ddd, 1H), 7.89 (br. s., 1H), 7.95 (ddd, 1H), 8.22 (d, 1H), 8.37 (br. s., 1H), 8.53 (s, 1H), 8.71 (d, 1H).

### Step 3: 2-carbamoylquinoline-4-carboxylic acid

In analogy to step 4) of intermediate 2A), 650 mg (2.82 mmol) methyl 2-carbamoylquinoline-4-carboxylate of step 2) of intermediate 3A) were reacted to give 540 mg (86%) of the desired title compound.

¹H-NMR (400 MHz, DMSO d₆) δ (ppm)=7.82 (dt, 1H), 7.86 (br. s., 1H), 7.92 (td, 1H), 8.20 (d, 1H), 8.34 (br. s., 1H), 8.50 (s, 1H), 8.78 (d, 1H), 13.98 (br. s., 1H).

Intermediate 4A

### 6,8-dichloro-2-(trifluoromethyl)quinoline-4-carboxylic acid

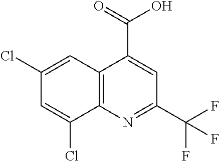 [SMILES: O=C(O)c1cc(C(F)(F)F)nc2c(Cl)cc(Cl)cc12]

In analogy to intermediate 1A, 1 g (4.63 mmol) 5,7-dichloro-1H-indole-2,3-dione was heated with 830 μL (9.26 mmol) 1,1,1-trifluoroacetone, 286 mg (5.10 mmol) potassium hydroxide, 530 μL (9.26 mmol) acetic acid and 531 mg (6.48 mmol) sodium acetate in 10 mL water for 2 h at 120° C. in the microwave to obtain 1.40 g (4.52 mmol, 98%) of the desired title compound after aqueous work-up.

1H NMR (300 MHz, DMSO d₆): δ (ppm)=8.39 (d, 1H), 8.42 (s, 1H), 8.87 (d, 1H).

Intermediate 5A

### 6,7-difluoro-2-(trifluoromethyl)quinoline-4-carboxylic acid

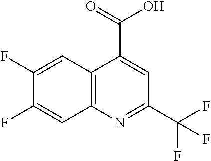 [SMILES: O=C(O)c1cc(C(F)(F)F)nc2cc(F)c(F)cc12]

In analogy to intermediate 1A, 265 mg (1.45 mmol) 5,6-difluoro-1H-indole-2,3-dione was heated with 259 μL (2.89 mmol) 1,1,1-trifluoroacetone, 89 mg (1.59 mmol) potassium hydroxide, 166 μL (2.89 mmol) acetic acid and 166 mg (2.03 mmol) sodium acetate in 2.7 mL water for 1 h at 120° C. in the microwave. As the conversion was not complete further 259 μL (2.89 mmol) 1,1,1-trifluoroacetone was added to the reaction mixture and heated again for 1 h at 120° C. in the microwave to obtain 312 mg (1.13 mmol, 78%) of the desired title compound after aqueous work-up.

1H NMR (300 MHz, DMSO d₆): δ (ppm)=8.33 (s, 1H), 8.41 (dd, 1H), 8.81 (dd, 1H), 14.62 (br. s., 1H).

Intermediate 6A

### 2-cyclopropyl-6-fluoroquinoline-4-carboxylic acid

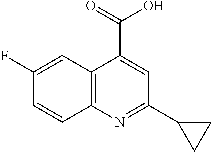 [SMILES: O=C(O)c1cc(C2CC2)nc2ccc(F)cc12]

In analogy to intermediate 1A, 300 mg (1.82 mmol) 5-fluoro-1H-indole-2,3-dione was heated with 900 μL (9.08 mmol) 1-cyclopropylethanone, 112 mg (2.00 mmol) potassium hydroxide, 208 μL (3.63 mmol) acetic acid and 209 mg (2.54 mmol) sodium acetate in 3 mL water for 24 h to reflux. The reaction mixture was filtered, the filtrate extracted with ethyl acetate and the combined organic layers were dried over sodium sulfate, filtered and evaporated to obtain 381 mg (1.65 mmol, 90%) of the desired title compound after drying.

1H NMR (400 MHz, DMSO d₆): δ (ppm)=0.54-0.64 (m, 1H), 0.66-0.83 (m, 3H), 1.92-2.04 (m, 1H), 6.09 (br. s., 1H), 6.74 (dd, 1H), 6.98 (ddd, 1H), 7.16 (dd, 1H), 10.19 (s, 1H).

Intermediate 7A

### 2-carbamoyl-6-chloro-7-fluoroquinoline-4-carboxylic acid

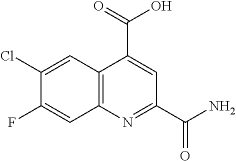 [SMILES: NC(=O)c1cc(C(=O)O)c2cc(Cl)c(F)cc2n1]

### Step 1: 6-chloro-7-fluoroquinoline-2,4-dicarboxylic acid

In analogy to step 1) of intermediate 2A), 10.0 g (50.1 mmol) commercially available 5-chloro-6-fluoro-1H-indole-2,3-dione were reacted to give 3.63 g (25%) 6-chloro-7-fluoroquinoline-2,4-dicarboxylic acid.

¹H-NMR (400 MHz, DMSO d₆) δ (ppm)=8.14 (d, 1H), 8.38 (s, 1H), 9.19 (d, 1H).

### Step 2: dimethyl 6-chloro-7-fluoroquinoline-2,4-dicarboxylate

In analogy to step 2) of intermediate 2A), 3.63 g (13.5 mmol) 6-chloro-7-fluoroquinoline-2,4-dicarboxylic acid of step 1) of intermediate 7A) were reacted to give 3.12 g (74%) dimethyl 6-chloro-7-fluoroquinoline-2,4-dicarboxylate.

¹H-NMR (500 MHz, DMSO d₆) δ (ppm)=3.98 (s, 3H), 4.01 (s, 3H), 8.29 (d, 1H), 8.46 (s, 1H), 8.94 (d, 1H).

### Step 3: methyl 2-carbamoyl-6-chloro-7-fluoroquinoline-4-carboxylate

In analogy to step 3) of intermediate 2A), 3.12 g (10.5 mmol) dimethyl 6-chloro-7-fluoroquinoline-2,4-dicarboxylate of step 2) of intermediate 7A) were reacted to give 2.52 g (77%) methyl 2-carbamoyl-6-chloro-7-fluoroquinoline-4-carboxylate.

¹H-NMR (400 MHz, DMSO d₆) δ (ppm)=4.01 (s, 3H), 7.98 (br. s., 1H), 8.11 (d, 1H), 8.36 (br. s., 1H), 8.54 (s, 1H), 8.96 (d, 1H).

### Step 4: 2-carbamoyl-6-chloro-7-fluoroquinoline-4-carboxylic acid

In analogy to step 4) of intermediate 2A), 520 mg (1.84 mmol) methyl 2-carbamoyl-6-chloro-7-fluoroquinoline-4-carboxylate of step 3) of intermediate 7A) were reacted to give 390 mg (63%) of the desired title compound.

¹H-NMR (400 MHz, DMSO d₆) δ (ppm)=7.92 (br. s., 1H), 8.06 (d, 1H), 8.31 (br. s., 1H), 8.51 (s, 1H), 9.10 (d, 1H).

Intermediate 1B

### 1-(4-fluorobenzyl)-5-methyl-4-nitro-1H-pyrazole and 1-(4-fluorobenzyl)-3-methyl-4-nitro-1H-pyrazole

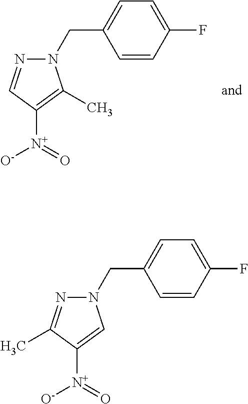 [SMILES: Cc1c([N+](=O)[O-])cnn1Cc1ccc(F)cc1 ; Cc1nn(Cc2ccc(F)cc2)cc1[N+](=O)[O-]]

1.0 g (7.87 mmol) 3-methyl-4-nitro-1H-pyrazole (CAS-No. 5334-39-4) was dissolved in 20 mL DMSO and 1.78 g (9.44 mmol) 1-(bromomethyl)-4-fluorobenzene and 1.76 mL (11.8 mmol) DBU were added. The suspension was stirred at rt for 2 h. Afterwards the reaction mixture was diluted with ethyl acetate. The organic phase was washed with water and brine, dried over sodium sulfate, filtered and evaporated to dryness. The crude product was purified via a Biotage chromatography system (50 g snap KP-Sit column, hexane/30-100% ethyl acetate). Using this methodology we obtained 1.72 g (93%) of the desired title compounds as a mixture.

1H NMR (400 MHz, DMSO d₆): δ (ppm)=2.40/2.62 (s, 3H), 5.31/5.43 (s, 2H), 7.16-7.25 (m, 2H), 7.26-7.34/7.36-7.46 (m, 2H), 8.28/8.96 (s, 1H).

Intermediate 2B

### 1-(3-fluorobenzyl)-5-methyl-4-nitro-1H-pyrazole and 1-(3-fluorobenzyl)-3-methyl-4-nitro-1H-pyrazole

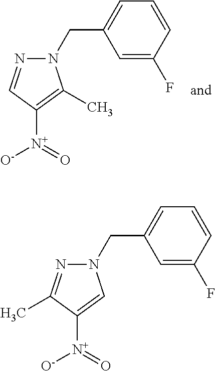 [SMILES: Cc1c([N+](=O)[O-])cnn1Cc1cccc(F)c1 ; Cc1nn(Cc2cccc(F)c2)cc1[N+](=O)[O-]]

In analogy to intermediate 1B, 1.0 g (7.63 mmol) 3-methyl-4-nitro-1H-pyrazole pyrazole and 1.6 g (8.40 mmol) 1-(bromomethyl)-3-fluorobenzene were reacted to give after purification of the crude product via a Biotage chromatography system (25 g snap KP-Sil column, hexane/0-100% ethyl acetate, then ethyl acetate/0-100% methanol) 587 mg (33%) of the desired title compounds as a mixture.

1H NMR (400 MHz, DMSO d₆): δ (ppm)=2.41/2.62 (s, 3H), 5.34/5.47 (s, 2H), 7.02-7.09 (m, 1H), 7.13-7.22 (m, 2H), 7.38-7.47 (m, 1H), 8.30/8.98 (s, 1H).

Intermediate 3B

### 1-(3,4-difluorobenzyl)-5-methyl-4-nitro-1H-pyrazole and 1-(3,4-difluorobenzyl)-3-methyl-4-nitro-1H-pyrazole

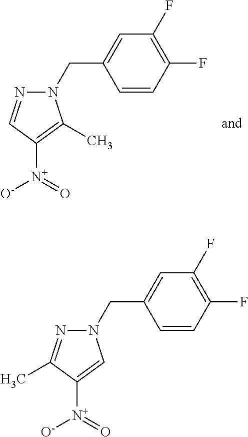 [SMILES: Cc1c([N+](=O)[O-])cnn1Cc1ccc(F)c(F)c1 ; Cc1nn(Cc2ccc(F)c(F)c2)cc1[N+](=O)[O-]]

In analogy to intermediate 1B, 1.0 g (7.63 mmol) 3-methyl-4-nitro-1H-pyrazole pyrazole and 1.77 g (8.40 mmol) 1-(bromomethyl)-3,4-difluorobenzene were reacted to give after purification of the crude product via a Biotage chromatography system (25 g snap KP-Sil column, hexane/0-100% ethyl acetate, then ethyl acetate/0-100% methanol) 1.77 mg (91%) of the desired title compounds as a mixture.

1H NMR (300 MHz, DMSO d₆): δ (ppm)=2.39/2.61 (s, 3H), 5.30/5.42 (s, 2H), 7.03-7.11/7.15-7.23 (m, 1H), 7.28-7.50 (m, 2H), 8.28/8.95 (s, 1H).

Intermediate 4B

### 4-[(5-methyl-4-nitro-1H-pyrazol-1-yl)methyl]benzonitrile and 4-[(3-methyl-4-nitro-1H-pyrazol-1-yl)methyl]benzonitrile

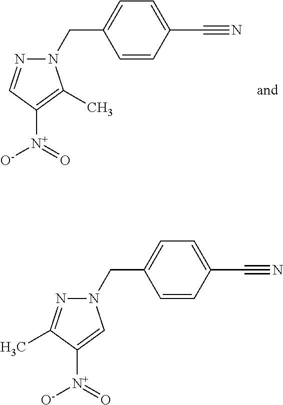 [SMILES: Cc1c([N+](=O)[O-])cnn1Cc1ccc(C#N)cc1 ; Cc1nn(Cc2ccc(C#N)cc2)cc1[N+](=O)[O-]]

5.00 g (39.4 mmol) 3-methyl-4-nitro-1H-pyrazole was dissolved in 115 mL acetonitrile and 9.26 g (47.2 mmol) 4-(bromomethyl)-benzonitrile and 15.4 g (47.2 mmol) cesium carbonate were added. The suspension was stirred at 60° C. for 3 h. Afterwards the reaction mixture was filtered, and the filter cake was washed with ethyl acetate. The filtrate was evaporated to dryness and the residue was purified via a Biotage chromatography system (100 g snap KP-Sil column, hexane/40-100% ethyl acetate) to give 7.27 g (76%) of the desired title compounds as a mixture.

¹H-NMR (400 MHz, DMSO d₆) δ (ppm)=2.39/2.59 (s, 3H), 5.42/5.55 (s, 2H), 7.35/7.47 (d, 2H), 7.80-7.85 (m, 2H), 8.29/8.99 (s, 1H).

Intermediate 5B

### 2-[(5-methyl-4-nitro-1H-pyrazol-1-yl)methyl]benzonitrile and 2-[(3-methyl-4-nitro-1H-pyrazol-1-yl)methyl]benzonitrile

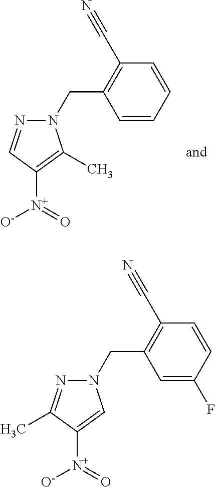 [SMILES: Cc1c([N+](=O)[O-])cnn1Cc1ccccc1C#N ; Cc1nn(Cc2cc(F)ccc2C#N)cc1[N+](=O)[O-]]

In analogy to intermediate 4B), 2.5 g (19.7 mmol) 3-methyl-4-nitro-1H-pyrazole and 4.6 g (23.6 mmol) 2-(bromomethyl)-benzonitrile were reacted to give after purification of the crude product via a Biotage chromatography system (50 g snap KP-Sil column, hexane/10-100% ethyl acetate, then ethyl acetate/0-25% methanol) 4.8 g (100%) of the desired title compounds as a mixture.

¹H-NMR (300 MHz, DMSO d₆) δ (ppm)=2.39/2.68 (s, 3H), 5.54 (s, 2H), 5.60 (s, 1H), 7.25 (d, 1H), 7.41 (d, 1H), 7.50-7.59 (m, 2H), 7.64-7.77 (m, 2H), 7.89 (d, 1H), 8.28 (s, 1H), 8.98 (s, 1H).

Intermediate 6B

### 1-(4-methoxybenzyl)-5-methyl-4-nitro-1H-pyrazole and 1-(4-methoxybenzyl)-3-methyl-4-nitro-1H-pyrazole

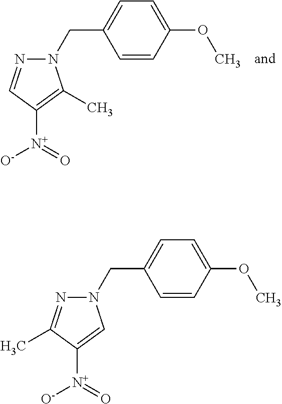

In analogy to intermediate 4B), 2.5 g (19.7 mmol) 3-methyl-4-nitro-1H-pyrazole and 3.70 g (23.6 mmol) 1-(bromomethyl)-4-methoxybenzene were reacted to give after purification of the crude product via a Biotage chromatography system (50 g snap KP-Sit column, hexane/10-100% ethyl acetate, then ethyl acetate/0-25% methanol) 4.9 g (100%) of the desired title compounds as a mixture.

¹H-NMR (300 MHz, DMSO d₆) δ (ppm)=2.38/2.60 (s, 3H), 3.72/3.72 (s, 3H), 5.21/5.34 (s, 2H), 6.85-6.94 (m, 2H), 7.18/7.29 (d, 2H), 8.24/8.89 (s, 1H).

Intermediate 7B

### 5-methyl-1-(4-methylbenzyl)-4-nitro-1H-pyrazole and 3-methyl-1-(4-methylbenzyl)-4-nitro-1H-pyrazole

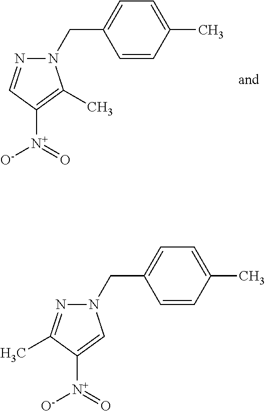

In analogy to intermediate 1B, 1.0 g (7.63 mmol) 3-methyl-4-nitro-1H-pyrazole pyrazole and 1.6 g (8.40 mmol) 4-methylbenzyl bromide were reacted to give after purification of the crude product via a Biotage chromatography system (25 g snap KP-Sit column, hexane/0-100% ethyl acetate, then ethyl acetate/0-100% methanol) 1.61 g (91%) of the desired title compounds as a mixture.

¹H-NMR (400 MHz, DMSO d₆) δ (ppm)=2.28/2.29 (s, 3H), 2.40/2.60 (s, 3H), 5.26/5.39 (s, 2H), 7.09-7.25 (m, 4H), 8.27/8.93 (s, 1H).

Intermediate 8B

### 4-[(3,5-dimethyl-4-nitro-1H-pyrazol-1-yl)methyl]pyridine and 4-[(3,5-dimethyl-4-nitro-1H-pyrazol-1-yl)methyl]pyridine

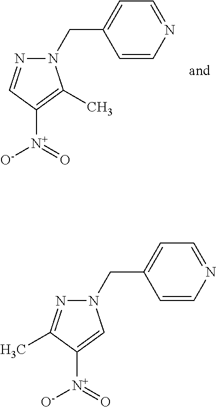 [SMILES: Cc1c([N+](=O)[O-])cnn1Cc1ccncc1 ; Cc1nn(Cc2ccncc2)cc1[N+](=O)[O-]]

In analogy to intermediate 1B, 1.0 g (7.63 mmol) 3-methyl-4-nitro-1H-pyrazole pyrazole, 4.1 g (16.0 mmol) 4-(bromomethyl)pyridine hydrobromide and 3.42 ml (22.9 mmol) DBU were reacted to give after purification of the crude product via a Biotage chromatography system (25 g snap KP-Sil column, hexane/0-100% ethyl acetate, then ethyl acetate/0-100% methanol) 220 mg (13%) of the desired title compounds as a mixture.

¹H-NMR (400 MHz, DMSO d₆) δ (ppm)=2.42/2.60 (s, 3H), 5.40/5.53 (s, 2H), 7.14/7.24 (d, 2H), 8.53-8.59 (m, 2H), 8.33/9.01 (s, 1H).

Intermediate 9B

### 1-(cyclohexylmethyl)-5-methyl-4-nitro-1H-pyrazole and 1-(cyclohexylmethyl)-3-methyl-4-nitro-1H-pyrazole

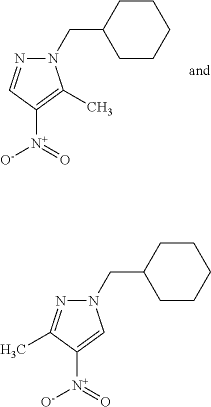 [SMILES: Cc1c([N+](=O)[O-])cnn1CC1CCCCC1 ; Cc1nn(CC2CCCCC2)cc1[N+](=O)[O-]]

In analogy to intermediate 1B, 1.0 g (7.63 mmol) 3-methyl-4-nitro-1H-pyrazole pyrazole and 1.5 g (8.40 mmol) (bromomethyl)-cyclohexane were reacted to give after purification of the crude product via a Biotage chromatography system (25 g snap KP-Sil column, hexane/0-100% ethyl acetate, then ethyl acetate/0-100% methanol) 1.47 g (86%) of the desired title compounds as a mixture.

¹H-NMR (300 MHz, DMSO d₆) δ (ppm)=0.83-1.26 and 1.43-1.89 (m, 10H), 2.41/2.59 (s, 3H), 3.92/3.97 (d, 2H), 8.21/8.78 (s, 1H).

Intermediate 10B

### methyl 1-(4-fluorobenzyl)-4-nitro-1H-pyrazole-5-carboxylate and methyl 1-(4-fluorobenzyl)-4-nitro-1H-pyrazole-3-carboxylate

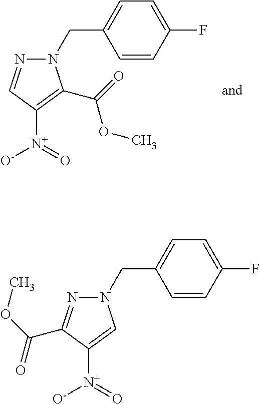 [SMILES: COC(=O)c1c([N+](=O)[O-])cnn1Cc1ccc(F)cc1 ; COC(=O)c1nn(Cc2ccc(F)cc2)cc1[N+](=O)[O-]]

In analogy to intermediate 4B), 1.0 g (5.84 mmol) methyl 4-nitro-1H-pyrazole-5-carboxylate (preparation described in Russ. Chem. Bull. Vol 42, No. 11, 1993 1861-1864) and 1.33 g (7.01 mmol) 1-(bromomethyl)-4-fluorobenzene were reacted to give after purification of the crude product via a Biotage chromatography system (50 g snap KP-Sil column, hexane/0-100% ethyl acetate) 1.22 g (71%) of polar isomer methyl 1-(4-fluorobenzyl)-4-nitro-1H-pyrazole-3-carboxylate and 0.46 g (27%) of unpolar isomer methyl 1-(4-fluorobenzyl)-4-nitro-1H-pyrazole-5-carboxylate.

NMR of Desired Compound

¹H-NMR (400 MHz, DMSO d₆) δ (ppm)=3.95 (s, 3H), 5.51 (s, 2H), 7.18-7.27 (m, 2H), 7.30-7.38 (m, 2H), 8.44 (s, 1H).

NMR of the Regiosiomer

¹H-NMR (400 MHz, DMSO d₆) δ (ppm)=3.87 (s, 3H), 5.43 (s, 2H), 7.18-7.30 (m, 2H), 7.41-7.49 (m, 2H), 9.15 (s, 1H).

Intermediate 11B

### methyl 1-(4-cyanobenzyl)-4-nitro-1H-pyrazole-5-carboxylate and methyl 1-(4-cyanobenzyl)-4-nitro-1H-pyrazole-3-carboxylate

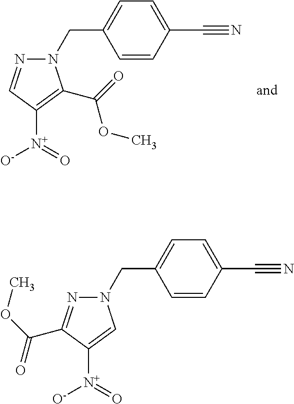 [SMILES: COC(=O)c1c([N+](=O)[O-])cnn1Cc1ccc(C#N)cc1 ; COC(=O)c1nn(Cc2ccc(C#N)cc2)cc1[N+](=O)[O-]]

In analogy to intermediate 4B), 1.0 g (5.84 mmol) methyl 4-nitro-1H-pyrazole-5-carboxylate (preparation see EP1953148) and 1.38 g (7.01 mmol) 4-(bromomethyl)-benzonitrile were reacted to give after twofold purification of the crude product via a Biotage chromatography system (50 g snap KP-Sil column, hexane/0-100% ethyl acetate) 1.02 g (58%) of polar isomer methyl 1-(4-cyanobenzyl)-4-nitro-1H-pyrazole-3-carboxylate and 0.43 g (24%) of unpolar isomer methyl 1-(4-cyanobenzyl)-4-nitro-1H-pyrazole-5-carboxylate.

NMR of Desired Compound

¹H-NMR (400 MHz, DMSO d₆) δ (ppm)=3.93 (s, 3H), 5.65 (s, 2H), 7.42 (d, 2H), 7.86 (d, 2H), 8.48 (s, 1H).

NMR of the Regiosiomer

¹H-NMR (400 MHz, DMSO d₆) δ (ppm)=3.87 (s, 3H), 5.57 (s, 2H), 7.53 (d, 2H), 7.88 (d, 2H), 9.19 (s, 1H).

Intermediate 12B

### 5-methyl-1-[(1-methyl-1H-pyrazol-3-yl)methyl]-4-nitro-1H-pyrazole and 3-methyl-1-[(1-methyl-1H-pyrazol-3-yl)methyl]-4-nitro-1H-pyrazole

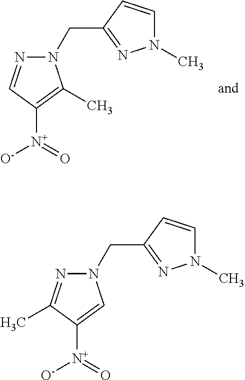

In analogy to intermediate 1B, 2.0 g (15.7 mmol) 3-methyl-4-nitro-1H-pyrazole and 2.47 g (18.9 mmol) 3-(chloromethyl)-1-methyl-1H-pyrazole (CAS-No. 84547-64-8) were reacted to give after purification of the crude product by flash chromatography 3.62 g (100%) of the desired title compounds as a 65:35 mixture of regioisomers.

1H NMR (300 MHz, DMSO d₆): δ (ppm)=2.39/2.65 (s, 3H), 3.80/3.78 (s, 3H), 5.24/5.32 (s, 2H), 6.23/6.14 (d, 1H), 7.64/7.63 (d, 1H), 8.62/8.21 (s, 1H).

Intermediate 13B

### 5-methyl-3-[(5-methyl-4-nitro-1H-pyrazol-1-yl)methyl]-1,2-oxazole and 5-methyl-3-[(3-methyl-4-nitro-1H-pyrazol-1-yl)methyl]-1,2-oxazole

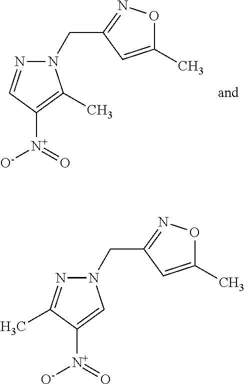

In analogy to intermediate 1B, 2.0 g (15.7 mmol) 3-methyl-4-nitro-1H-pyrazole and 2.48 g (18.9 mmol) 3-(chloromethyl)-5-methyl-1,2-oxazole (CAS-No. 35166-37-1) were reacted to give after purification of the crude product by flash chromatography 1.19 g (34%) of the desired title compounds as a 60:40 mixture of regioisomers.

1H NMR (300 MHz, DMSO d₆): δ (ppm)=2.37/2.37 (s, 3H), 2.40/2.63 (s, 3H), 5.39/5.50 (s, 2H), 6.21/6.16 (br.s., 1H), 8.95/8.27 (s, 1H).

Intermediate 14B

### 3-ethyl-5-[(5-methyl-4-nitro-1H-pyrazol-1-yl)methyl]-1,2,4-oxadiazole and 3-ethyl-5-[(3-methyl-4-nitro-1H-pyrazol-1-yl)methyl]-1,2,4-oxadiazole

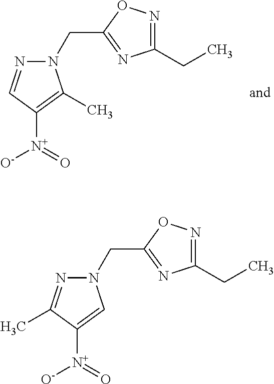

In analogy to intermediate 1B, 2.0 g (15.7 mmol) 3-methyl-4-nitro-1H-pyrazole and 2.77 g (18.9 mmol) 5-(chloromethyl)-3-ethyl-1,2,4-oxadiazole (CAS-No. 64988-69-8) were reacted to give after purification of the crude product by flash chromatography 1.93 g (52%) of the desired title compounds as a 70:30 mixture of regioisomers.

1H NMR (400 MHz, DMSO d₆): δ (ppm)=1.20/1.22 (t, 3H), 2.66/2.43 (s, 3H), 2.71/2.72 (q, 2H), 5.91/5.80 (s, 2H), 8.32/9.00 (s, 1H).

Intermediate 15B

### N-methyl-3-[(5-methyl-4-nitro-1H-pyrazol-1-yl)methyl]-1,2,4-oxadiazole-5-carboxamide and N-methyl-3-[(3-methyl-4-nitro-1H-pyrazol-1-yl)methyl]-1,2,4-oxadiazole-5-carboxamide

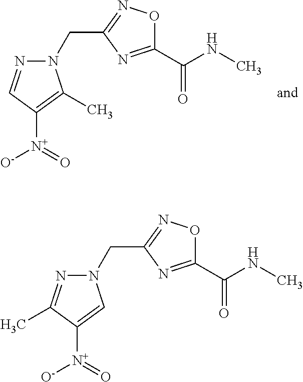

In analogy to intermediate 1B, 2.0 g (15.7 mmol) 3-methyl-4-nitro-1H-pyrazole and 3.32 g (18.9 mmol) 3-(chloromethyl)-N-methyl-1,2,4-oxadiazole-5-carboxamide (CAS-No. 1123169-42-5) were reacted to give after purification of the crude product by flash chromatography 3.32 g (79%) of the desired title compounds as a 60:40 mixture of regioisomers.

1H NMR (400 MHz, DMSO d₆): δ (ppm)=2.42/2.69 (s, 3H), 2.78 (d, 3H), 5.67/5.77 (s, 2H), 9.00/8.30 (s, 1H), 9.30/9.26 (br.q., 1H).

Intermediate 16B

### 2-methyl-4-[(5-methyl-4-nitro-1H-pyrazol-1-yl)methyl]-1,3-thiazole and 2-methyl-4-[(3-methyl-4-nitro-1H-pyrazol-1-yl)methyl]-1, 3-thiazole

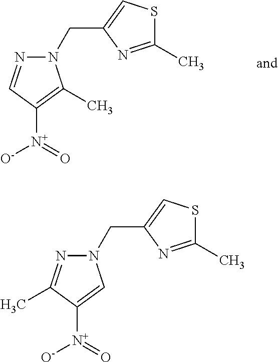

In analogy to intermediate 1B, 2.0 g (15.7 mmol) 3-methyl-4-nitro-1H-pyrazole and 3.48 g (18.9 mmol) 4-(chloromethyl)-2-methyl-1,3-thiazole hydrochloride (1:1) (CAS-No. 77470-53-2) were reacted to give after purification of the crude product by flash chromatography 2.47 g (79%) of the desired title compounds as a 55:45 mixture of regioisomers.

Intermediate 17B

### tert-butyl 4-[(5-methyl-4-nitro-1H-pyrazol-1-yl)methyl]piperidine-1-carboxylate and tert-butyl 4-[(3-methyl-4-nitro-1H-pyrazol-1-yl)methyl]piperidine-1-carboxylate

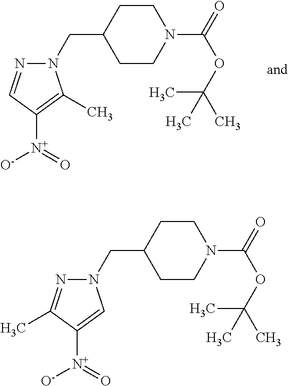

In analogy to intermediate 1B, 381 mg (3.00 mmol) 3-methyl-4-nitro-1H-pyrazole and 1.00 g (3.60 mmol) tert-butyl 4-(bromomethyl)piperidine-1-carboxylate (CAS-No. 158407-04-6) were reacted to give after purification of the crude product by flash chromatography 940 mg (97%) of the desired title compounds as a 60:40 mixture of regioisomers.

1H NMR (400 MHz, DMSO d₆): δ (ppm)=1.05/1.11 (m, 2H), 1.38 (s, 9H), 1.46 (m, 2H), 2.01 (m, 1H), 2.42/2.61 (s, 3H), 2.67 (m, 2H), 3.92 (m, 2H), 4.00/4.05 (d, 2H), 8.78/8.23 (s, 1H).

Intermediate 18B

### 4-[(5-methyl-4-nitro-1H-pyrazol-1-yl)methyl]piperidine and 4-[(3-methyl-4-nitro-1H-pyrazol-1-yl)methyl]piperidine

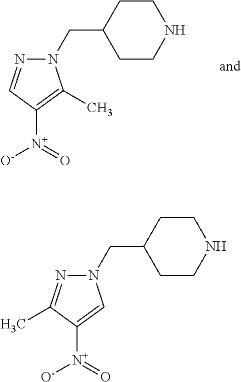 [SMILES: Cc1c([N+](=O)[O-])cnn1CC1CCNCC1 ; Cc1nn(CC2CCNCC2)cc1[N+](=O)[O-]]

A solution of 940 mg (2.90 mmol) tert-butyl 4-[(5-methyl-4-nitro-1H-pyrazol-1-yl)methyl]piperidine-1-carboxylate and tert-butyl 4-[(3-methyl-4-nitro-1H-pyrazol-1-yl)methyl]piperidine-1-carboxylate (intermediate 17B) in 5 mL dichloromethane was stirred with 2.2 mL (29.0 mmol) trifluoroacetic acid for three hours. The reaction mixture was filtered over NH₂ derivatized silica gel, and the filtrate was evaporated yielding 557 mg of the desired title compounds as crude product which was used without further purification.

1H NMR (400 MHz, DMSO d₆): δ (ppm)=1.04/1.11 (m, 2H), 1.39 (m, 2H), 1.89 (m, 1H), 2.38 (m, 2H), 2.42/2.60 (s, 3H), 2.90 (m, 2H), 3.95/4.01 (d, 2H), 8.80/8.23 (s, 1H).

Intermediate 19B

### 1-(ethylsulfonyl)-4-[(5-methyl-4-nitro-1H-pyrazol-1-yl)methyl]piperidine and 1-(ethylsulfonyl)-4-[(3-methyl-4-nitro-1H-pyrazol-1-yl)methyl]piperidine

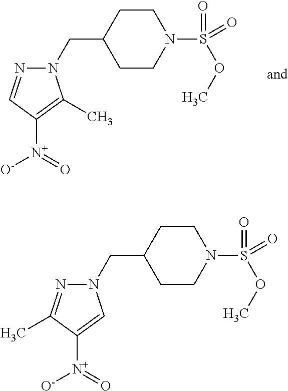 [SMILES: COS(=O)(=O)N1CCC(Cn2ncc([N+](=O)[O-])c2C)CC1 ; COS(=O)(=O)N1CCC(Cn2cc([N+](=O)[O-])c(C)n2)CC1]

A solution of 550 mg (2.45 mmol) 4-[(5-methyl-4-nitro-1H-pyrazol-1-yl)methyl]piperidine and 4-[(3-methyl-4-nitro-1H-pyrazol-1-yl)methyl]piperidine (intermediate 18B) in 5 mL DMF was stirred with 195 μL (2.06 mmol) ethanesulfonyl chloride and 1.23 mL (8.83 mmol) triethylamine overnight. Saturated aqueous sodium bicarbonate was added to the reaction. The mixture was extracted with ethyl acetate, and the combined organic phase was washed with brine, dried, filtered, and evaporated. 628 mg of the desired title compounds as crude product were obtained which were used without further purification.

1H NMR (400 MHz, DMSO d₆): δ (ppm)=1.19 (t, 3H), 1.20/1.27 (m, 2H), 1.57 (m, 2H), 2.00 (m, 1H), 2.42/2.62 (s, 3H), 2.76 (m, 2H), 3.01 (q, 2H), 3.58 (m, 2H), 4.03/4.09 (d, 2H), 8.80/8.24 (s, 1H).

Intermediate 20B

### 4-[(3-ethyl-4-nitro-1H-pyrazol-1-yl)methyl]benzonitrile and 4-[(5-ethyl-4-nitro-1H-pyrazol-1-yl)methyl]benzonitrile

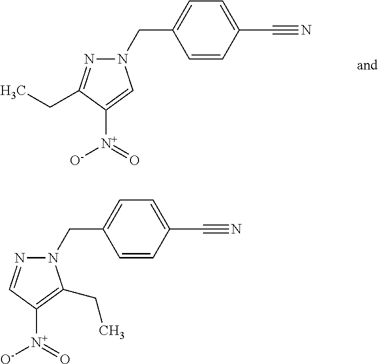

In analogy to intermediate 1B, 2.49 g (17.6 mmol) 4-nitro-3-(ethyl)-1H-pyrazole (CAS-No 70951-91-6, commercially available e.g. Accel Pharmtech LLC, Advanced ChemBlocks Inc. or Tractus Company Limited), 4.15 g (21.2 mmol) 4-(bromomethyl)-benzonitrile and 3.9 ml (26 mmol) DBU were reacted to give after twofold purification of the crude product via a Biotage chromatography system (25 g snap KP-Sit column, hexane/0-90% ethyl acetate) 3.09 g (89%) of the desired title compounds as a mixture.

¹H-NMR (400 MHz, DMSO d₆) δ (ppm)=1.02/1.16 (t, 3H), 2.84/3.03 (q, 2H), 5.44/5.58 (s, 2H), 7.36/7.46 (d, 2H), 7.81-7.87 (m, 2H), 8.32/8.98 (s, 1H).

Intermediate 21B

### 4-[(3-isopropyl-4-nitro-1H-pyrazol-1-yl)methyl]benzonitrile and 4-[(5-isopropyl-4-nitro-1H-pyrazol-1-yl)methyl]benzonitrile

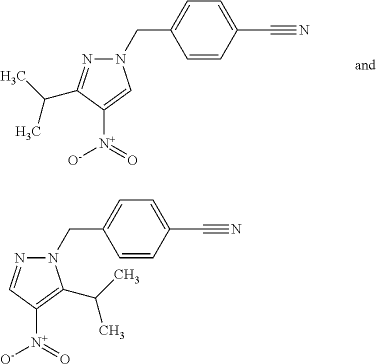

In analogy to intermediate 4B, 2.08 g (13.4 mmol) 4-nitro-3-(isopropyl)-1H-pyrazole (CAS-No 51355-77-2, commercially available e.g. Combi-Blocks Inc. or UkrOrgSynthesis Ltd.) and 2.19 g (21.2 mmol) 4-(bromomethyl)-benzonitrile were reacted to give after purification of the crude product via a Biotage chromatography system (25 g snap KP-Sil column, hexane/0-100% ethyl acetate, then ethyl acetate/0-15% methanol) 1.30 g (43%) of the desired title compounds as a mixture.

¹H-NMR (400 MHz, DMSO d₆) δ (ppm)=1.21/1.27 (d, 6H), 3.48/3.57 (quin, 1H), 5.47/5.67 (s, 2H), 7.32/7.446 (d, 2H), 7.84-7.89 (m, 2H), 8.99 (s, 1H).

Intermediate 1C

### 1-(4-fluorobenzyl)-5-methyl-1H-pyrazol-4-amine and 1-(4-fluorobenzyl)-3-methyl-1H-pyrazol-4-amine

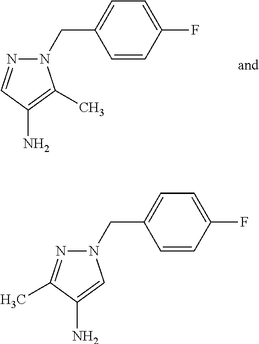

To a solution of 9.48 g (40.3 mmol) of 1-(4-fluorobenzyl)-5-methyl-4-nitro-1H-pyrazole and 1-(4-fluorobenzyl)-3-methyl-4-nitro-1H-pyrazole (intermediate 1B) in 211 mL ethanol was added 45.5 g (202 mmol) stannous chloride dihydrate. This reaction mixture was stirred at reflux for 2 hours and then at 70° C. for 18 hours. After cooling to 25° C. the mixture was evaporated. To the residue diluted with 250 ml ethyl acetate, 5M aq. sodium hydroxide solution was added to get a basic pH. The formed precipitate was separated by filtration and the separated aqueous phase was extracted three times with 150 mL ethyl acetate. The combined organic layers were washed with water, brine, dried over sodium sulfate, filtered and evaporated to obtain a crude product, which was purified via a Biotage chromatography system (100 g snap KP-Sil column, hexane/50-100% ethyl acetate, then ethyl acetate/0-40% methanol) to obtain 6.06 g (73%) of the desired title compounds as a mixture.

1H NMR (300 MHz, DMSO d₆): δ (ppm)=1.97/1.99 (s, 3H), 3.69 (br. s., 2H), 5.01/5.12 (s, 2H), 6.93/7.00 (s, 1H), 7.04-7.23 (m, 4H).

Intermediate 2C

### 1-(3-fluorobenzyl)-5-methyl-1H-pyrazol-4-amine and 1-(3-fluorobenzyl)-3-methyl-1H-pyrazol-4-amine

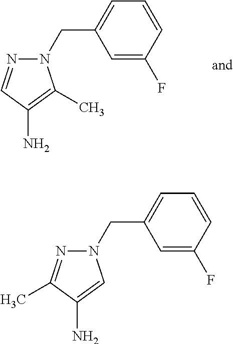

In analogy to intermediate 1C), 500 mg (2.13 mmol) 1-(3-fluorobenzyl)-5-methyl-4-nitro-1H-pyrazole and 1-(3-fluorobenzyl)-3-methyl-4-nitro-1H-pyrazole (intermediate 2B) were reacted to give after purification of the crude product via a Biotage chromatography system (25 g snap KP-Sil column, hexane/0-100% ethyl acetate, then ethyl acetate/0-100% methanol) 375 mg (86%) of the desired title compounds as a mixture.

1H NMR (400 MHz, DMSO d₆): δ (ppm)=2.00/2.01 (s, 3H), 3.69 (s, 2H), 5.07/5.18 (s, 2H), 6.79/6.93 (d, 1H), 6.88/6.99 (d, 1H), 6.97/7.05 (s, 1H), 7.05-7.12 (m, 1H), 7.31-7.41 (m, 1H).

Intermediate 3C

### 1-(3,4-difluorobenzyl)-5-methyl-1H-pyrazol-4-amine and 1-(3,4-difluorobenzyl)-3-methyl-1H-pyrazol-4-amine

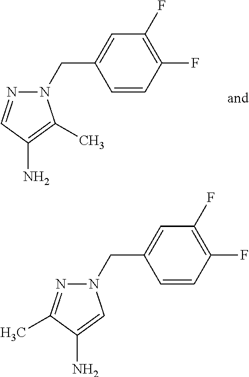 [SMILES: Cc1c(N)cnn1Cc1ccc(F)c(F)c1 ; Cc1nn(Cc2ccc(F)c(F)c2)cc1N]

In analogy to intermediate 1C), 500 mg (2.13 mmol) 1-(3,4-difluorobenzyl)-5-methyl-4-nitro-1H-pyrazole and 1-(3,4-difluorobenzyl)-3-methyl-4-nitro-1H-pyrazole (intermediate 3B) were reacted to give after purification of the crude product via a Biotage chromatography system (25 g snap KP-Sil column, hexane/0-100% ethyl acetate, then ethyl acetate/0-100% methanol) 251 mg (57%) of the desired title compounds as a mixture.

1H NMR (400 MHz, DMSO d₆): δ (ppm)=1.99/2.01 (s, 3H), 3.65 (s, 2H), 5.04/5.15 (s, 2H), 6.85-7.10 and 7.16-7.24 (m, 3H), 7.33-7.43 (m, 1H).

Intermediate 4C

### 4-[(4-amino-5-methyl-1H-pyrazol-1-yl)methyl]benzonitrile and 4-[(4-amino-3-methyl-1H-pyrazol-1-yl)methyl]benzonitrile

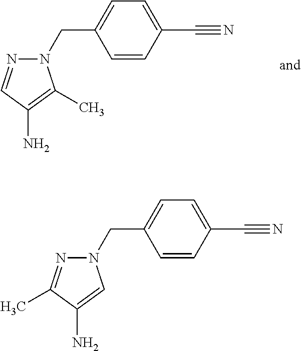 [SMILES: Cc1c(N)cnn1Cc1ccc(C#N)cc1 ; Cc1nn(Cc2ccc(C#N)cc2)cc1N]

In analogy to intermediate 1C), 7.27 g (30.0 mmol) 4-[(5-methyl-4-nitro-1H-pyrazol-1-yl)methyl]benzonitrile and 4-[(3-methyl-4-nitro-1H-pyrazol-1-yl)methyl]benzonitrile (intermediate 4B) were reacted to give after purification of the crude product via a Biotage chromatography system (100 g snap KP-Sil column, hexane/50-100% ethyl acetate, then ethyl acetate/0-40% methanol) 4.42 g (69%) of the desired title compounds as a mixture.

¹H-NMR (300 MHz, DMSO d₆) δ (ppm)=1.98 (s, 3H), 3.66 (br. s., 2H), 5.15/5.25 (s, 2H), 6.97/7.06 (s, 1H), 7.15/7.26 (d, 2H), 7.77 (d, 2H).

Intermediate 5C

### 2-[(4-amino-5-methyl-1H-pyrazol-1-yl)methyl]benzonitrile and 2-[(4-amino-3-methyl-1H-pyrazol-1-yl)methyl]benzonitrile

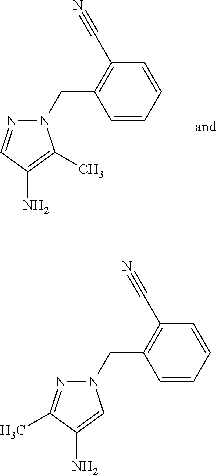 [SMILES: Cc1c(N)cnn1Cc1ccccc1C#N ; Cc1nn(Cc2ccccc2C#N)cc1N]

In analogy to intermediate 1C), 4.84 g (20.0 mmol) 2-[(5-methyl-4-nitro-1H-pyrazol-1-yl)methyl]benzonitrile and 2-[(3-methyl-4-nitro-1H-pyrazol-1-yl)methyl]benzonitrile (intermediate 5B) were reacted to give after purification of the crude product via a Biotage chromatography system (100 g snap KP-Sil column, hexane/80-100% ethyl acetate, then ethyl acetate/0-50% methanol) 2.70 g (64%) of the desired title compounds as a mixture.

¹H-NMR (400 MHz, DMSO d₆) δ (ppm)=1.97/1.98 (s, 3H), 3.68 (br. s., 2H), 5.24/5.31 (s, 2H), 6.88/7.13 (d, 1H), 6.97/7.07 (s, 1H), 7.42-7.51 (m, 1H), 7.60-7.68 (m, 1H), 7.80-7.87 (m, 1H).

Intermediate 6C

### 1-(4-methoxybenzyl)-5-methyl-1H-pyrazol-4-amine and 1-(4-methoxybenzyl)-3-methyl-1H-pyrazol-4-amine

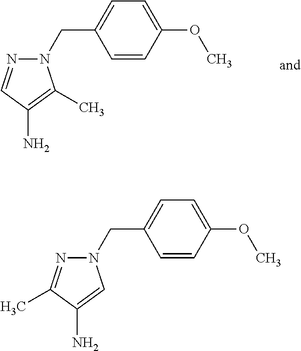

A mixture of 4.94 g (20.0 mmol) 1-(4-methoxybenzyl)-5-methyl-4-nitro-1H-pyrazole and 1-(4-methoxybenzyl)-3-methyl-4-nitro-1H-pyrazole (intermediate 6B) was dissolved in 78 mL methanol, and 522 mg palladium on carbon (10 wt. %) and 10.1 g (160 mmol) ammonium formate were added. The reaction mixture was heated for 1 h at 80° C. Afterwards the suspension was filtered through Celite and the filtrate was evaporated. The residue was diluted with 50 mL water and this phase was extracted three times with ethyl acetate. The combined organic phase was washed with brine, dried over sodium sulfate, filtered and evaporated to obtain a crude material which was purified via a Biotage chromatography system (100 g snap KP-Sil column, hexane/20-70% ethyl acetate) to give 3.64 g (84%) of the desired title compounds as a mixture.

¹H-NMR (400 MHz, DMSO d₆) δ (ppm)=1.96/1.98 (s, 3H), 3.55 (s, 2H), 3.70/3.71 (s, 3H), 4.94/5.05 (s, 2H), 6.83-6.88 (m, 2H), 6.90/6.94 (s, 1H), 6.98-7.02/7.09-7.14 (m, 2H).

Intermediate 7C

### 5-methyl-1-(4-methylbenzyl)-1H-pyrazol-4-amine and 3-methyl-1-(4-methylbenzyl)-1H-pyrazol-4-amine

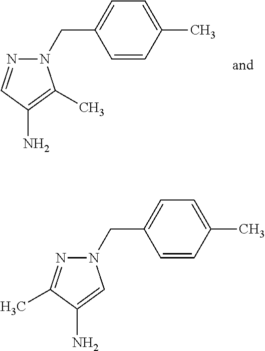

In analogy to intermediate 6C), 500 mg (2.160 mmol) of a mixture of 5-methyl-1-(4-methylbenzyl)-4-nitro-1H-pyrazole and 3-methyl-1-(4-methylbenzyl)-4-nitro-1H-pyrazole (intermediate 7B) were reacted to give after purification of the crude product via a Biotage chromatography system (25 g snap KP-Sil column, hexane/0-100% ethyl acetate, then ethyl acetate/0-100% methanol) 337 mg (78%) of the desired title compounds as a mixture.

¹H-NMR (400 MHz, DMSO d₆) δ (ppm)=1.96/1.97 (s, 3H), 2.24/2.25 (s, 3H), 3.57 (s, 2H), 4.96/5.08 (s, 2H), 6.88-6.96 (m, 2H), 7.02-7.14 (m, 3H).

Intermediate 8C

### 5-methyl-1-(pyridin-4-ylmethyl)-1H-pyrazol-4-amine and 3-methyl-1-(pyridin-4-ylmethyl)-1H-pyrazol-4-amine

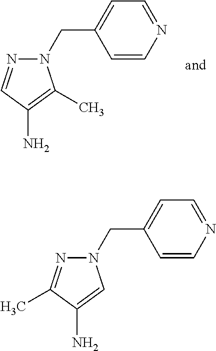

In analogy to intermediate 6C), 200 mg (0.92 mmol) of a mixture of 4-[(3,5-dimethyl-4-nitro-1H-pyrazol-1-yl)methyl]pyridine and 4-[(3,5-dimethyl-4-nitro-1H-pyrazol-1-yl)methyl]pyridine (intermediate 8B) were reacted to give only via filtration through Celite 141 mg (82%) of the desired title compounds as a mixture.

¹H-NMR (400 MHz, DMSO d₆) δ (ppm)=1.98/1.99 (s, 3H), 3.59-3.71 (m, 2H), 5.10/5.20 (s, 2H), 6.93-7.07 (m, 3H), 8.45-8.50 (m, 2H).

Intermediate 9C

### 1-(cyclohexylmethyl)-5-methyl-1H-pyrazol-4-amine and 1-(cyclohexylmethyl)-3-methyl-1H-pyrazol-4-amine

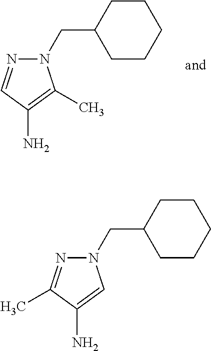

In analogy to intermediate 6C), 500 mg (2.24 mmol) of a mixture of 1-(cyclohexylmethyl)-5-methyl-4-nitro-1H-pyrazole and 1-(cyclohexylmethyl)-3-methyl-4-nitro-1H-pyrazole (intermediate 9B) were reacted to give after purification of the crude product via a Biotage chromatography system (25 g snap KP-Sil column, hexane/0-100% ethyl acetate, then ethyl acetate/0-100% methanol) 199 mg (46%) of the desired title compounds as a mixture.

¹H-NMR (400 MHz, DMSO d₆) δ (ppm)=0.81-1.23 (m, 5H), 1.43-1.76 (m, 6H), 1.96 (s, 3H), 3.50 (s, 2H), 3.63 (d, 1H), 6.88 (s, 1H).

Intermediate 10C

### methyl 4-amino-1-(4-fluorobenzyl)-1H-pyrazole-5-carboxylate

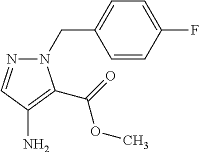 [SMILES: COC(=O)c1c(N)cnn1Cc1ccc(F)cc1]

To a solution of 460 mg (1.65 mmol) methyl 1-(4-fluorobenzyl)-4-nitro-1H-pyrazole-5-carboxylate (intermediate 10B) in 16.9 mL ethanol was added 8.4 mL water, 1.88 mL acetic acid and 377 mg (5.77 mmol) zinc dust. This reaction mixture was stirred at 60° C. for 2 hours. After cooling to 25° C. the suspension was filtered through Celite, washed with ethyl acetate and the complete filtrate was evaporated. To the residue 50 mL water and 40 mL of conc. aq. sodium carbonate was added. This aqueous phase was extracted three times with 100 mL ethyl acetate. The combined organic layer was washed with brine, dried over sodium sulfate, filtered and evaporated to obtain a crude product, which was purified via a Biotage chromatography system (50 g snap KP-Sil column, hexane/50-100% ethyl acetate then ethyl acetate/0-75% methanol) to obtain 300 mg (69%) of the desired title compound.

1H NMR (300 MHz, DMSO d₆): δ (ppm)=3.74 (s, 3H), 5.09 (s, 2H), 5.47 (s, 2H), 7.07-7.15 (m, 5H).

Intermediate 11C

### methyl 4-amino-1-(4-cyanobenzyl)-1H-pyrazole-5-carboxylate

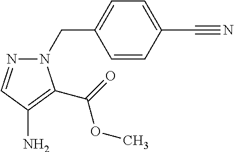 [SMILES: COC(=O)c1c(N)cnn1Cc1ccc(C#N)cc1]

In analogy to intermediate 10C), 430 mg (1.50 mmol) methyl 1-(4-cyanobenzyl)-4-nitro-1H-pyrazole-5-carboxylate (intermediate 11B) were reacted to give after purification of the crude product via a Biotage chromatography system (50 g snap KP-Sil column, hexane/50-100% ethyl acetate, then ethyl acetate/0-75% methanol) 270 mg (68%) of the desired title compound.

1H NMR (300 MHz, DMSO d₆): δ (ppm)=3.72 (s, 3H), 5.15 (s, 2H), 5.58 (s, 2H), 7.15 (s, 1H), 7.18 (d, 2H), 7.76 (d, 2H).

Intermediate 12C

### 5-methyl-1-[(1-methyl-1H-pyrazol-3-yl)methyl]-1H-pyrazol-4-amine and 3-methyl-1-[(1-methyl-1H-pyrazol-3-yl)methyl]-1H-pyrazol-4-amine

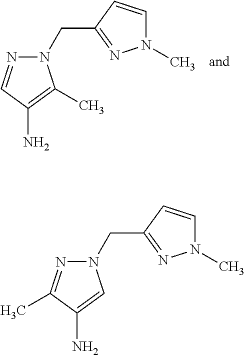

In analogy to intermediate 1C, 3.26 g (15.7 mmol) 5-methyl-1-[(1-methyl-1H-pyrazol-3-yl)methyl]-4-nitro-1H-pyrazole and 3-methyl-1-[(1-methyl-1H-pyrazol-3-yl)methyl]-4-nitro-1H-pyrazole (intermediate 12B) were reacted to give after purification of the crude product by flash chromatography 422 mg (14%) of the desired title compounds as a 65:35 mixture of regioisomers.

1H NMR (400 MHz, DMSO d₆): δ (ppm)=1.97/2.07 (s, 3H), 3.78/3.77 (s, 3H), 4.93/5.02 (s, 2H), 6.04/5.90 (d, 1H), 6.92/6.87 (s, 1H), 7.57/7.55 (d, 1H).

Intermediate 13C

### 5-methyl-1-[(5-methyl-1,2-oxazol-3-yl)methyl]-1H-pyrazol-4-amine and 3-methyl-1-[(5-methyl-1,2-oxazol-3-yl)methyl]-1H-pyrazol-4-amine

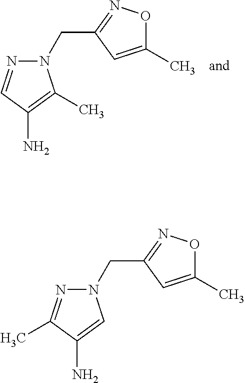

In analogy to intermediate 1C, 1.19 g (5.36 mmol) 5-methyl-3-[(5-methyl-4-nitro-1H-pyrazol-1-yl)methyl]-1,2-oxazole and 5-methyl-3-[(3-methyl-4-nitro-1H-pyrazol-1-yl)methyl]-1,2-oxazole (intermediate 13B) were reacted to give a crude product of 500 mg (49%) of the desired title compounds as a 60:40 mixture of regioisomers which was reacted further without purification.

Intermediate 14C

### 1-[(3-ethyl-1,2,4-oxadiazol-5-yl)methyl]-5-methyl-1H-pyrazol-4-amine and 1-[(3-ethyl-1,2,4-oxadiazol-5-yl)methyl]-3-methyl-1H-pyrazol-4-amine

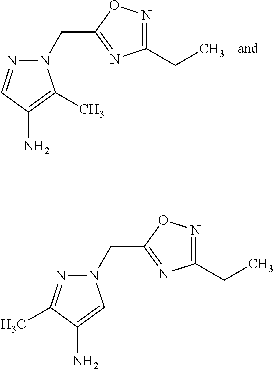

In analogy to intermediate 1C, 1.93 g (5.70 mmol) 3-ethyl-5-[(5-methyl-4-nitro-1H-pyrazol-1-yl)methyl]-1,2,4-oxadiazole and 3-ethyl-5-[(3-methyl-4-nitro-1H-pyrazol-1-yl)methyl]-1,2,4-oxadiazole (intermediate 14B) were reacted to give after purification of the crude product by flash chromatography 1.14 g (67%) of the desired title compounds as a 80:20 mixture of regioisomers.

1H NMR (400 MHz, DMSO d₆): δ (ppm)=1.17/1.19 (t, 3H), 1.99/2.26 (s, 3H), 2.70/2.71 (q, 2H), 5.70/5.63 (s, 2H), 7.39/7.77 (s, 1H).

Intermediate 15C

### 3-[(4-amino-5-methyl-1H-pyrazol-1-yl)methyl]-N-methyl-1,2,4-oxadiazole-5-carboxamide and 3-[(4-amino-3-methyl-1H-pyrazol-1-yl)methyl]-N-methyl-1,2,4-oxadiazole-5-carboxamide

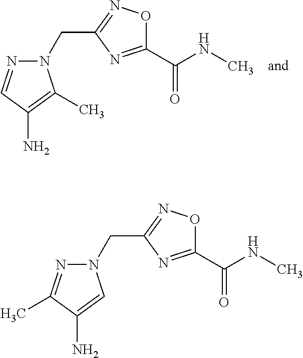

In analogy to intermediate 1C, 3.32 g (12.5 mmol)N-methyl-3-[(5-methyl-4-nitro-1H-pyrazol-1-yl)methyl]-1,2,4-oxadiazole-5-carboxamide and N-methyl-3-[(3-methyl-4-nitro-1H-pyrazol-1-yl)methyl]-1,2,4-oxadiazole-5-carboxamide (intermediate 15B) were reacted to give as crude product 2.80 g (95%) of the desired title compounds as a 55:45 mixture of regioisomers that were used further without purification.

1H NMR (400 MHz, DMSO d₆): δ (ppm)=2.78/2.78 (d, 3H), 2.31/2.11 (s, 3H), 5.57/5.53 (s, 2H), 7.40/7.81 (s, 1H), 9.30/9.31 (br.q., 1H).

Intermediate 16C

### 5-methyl-1-[(2-methyl-1,3-thiazol-4-yl)methyl]-1H-pyrazol-4-amine and 3-methyl-1-[(2-methyl-1,3-thiazol-4-yl)methyl]-1H-pyrazol-4-amine

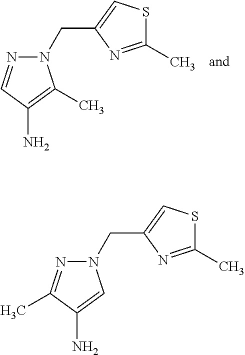

In analogy to intermediate 1C, 2.47 g (10.4 mmol) 2-methyl-4-[(5-methyl-4-nitro-1H-pyrazol-1-yl)methyl]-1,3-thiazole and 2-methyl-4-[(3-methyl-4-nitro-1H-pyrazol-1-yl)methyl]-1,3-thiazole (intermediate 16B) were reacted to give after purification of the crude product by flash chromatography 450 mg (21%) of the desired title compounds as a 55:45 mixture of regioisomers.

1H NMR (400 MHz, DMSO d₆): δ (ppm)=2.01/2.16 (s, 3H), 2.61/2.60 (s, 3H), 5.10/5.18 (s, 2H), 7.18/7.05 (s, 1H), 7.22/7.04 (s, 1H).

Intermediate 17C

### 1-{[1-(ethylsulfonyl)piperidin-4-yl]methyl}-5-methyl-1H-pyrazol-4-amine and 1-{[1-(ethylsulfonyl)piperidin-4-yl]methyl}-3-methyl-1H-pyrazol-4-amine

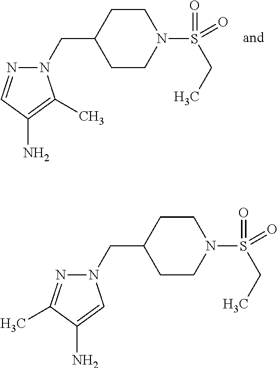 [SMILES: CCS(=O)(=O)N1CCC(Cn2ncc(N)c2C)CC1 ; CCS(=O)(=O)N1CCC(Cn2cc(N)c(C)n2)CC1]

In analogy to intermediate 1C, 628 mg (1.98 mmol) 1-(ethylsulfonyl)-4-[(5-methyl-4-nitro-1H-pyrazol-1-yl)methyl]piperidine and 1-(ethylsulfonyl)-4-[(3-methyl-4-nitro-1H-pyrazol-1-yl)methyl]piperidine (intermediate 19B) were reacted to give as crude product 661 mg of the desired title compounds as a 55:45 mixture of regioisomers that were used further without purification.

1H NMR (400 MHz, DMSO d₆): δ (ppm)=1.18 (t, 3H), 1.21/1.16 (m, 2H), 1.52 (m, 2H), 1.89 (m, 1H), 2.07/2.19 (s, 3H), 2.73 (m, 2H), 2.99 (q, 2H), 3.56 (m, 2H), 3.86/3.89 (d, 2H), 7.40/7.21 (s, 1H).

Intermediate 18C

### 4-[(4-amino-3-ethyl-1H-pyrazol-1-yl)methyl]benzonitrile and 4-[(4-amino-5-ethyl-1H-pyrazol-1-yl)methyl]benzonitrile

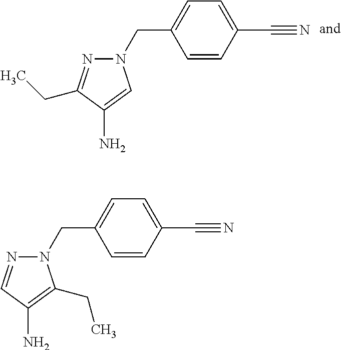 [SMILES: CCc1nn(Cc2ccc(C#N)cc2)cc1N ; CCc1c(N)cnn1Cc1ccc(C#N)cc1]

In analogy to intermediate 10C), in a first experiment 250 mg (0.98 mmol) and in a second experiment 2.84 g (11.1 mmol) of 4-[(3-ethyl-4-nitro-1H-pyrazol-1-yl)methyl]benzonitrile and 4-[(5-ethyl-4-nitro-1H-pyrazol-1-yl)methyl]benzonitrile (intermediate 20B) were reacted to give after purification of the combined crude products via a Biotage chromatography system (50 g snap KP-Sil column, hexane/0-100% ethyl acetate, then ethyl acetate/0-10% methanol) 1.60 g (59%) of the desired title compounds as a mixture.

¹H-NMR (500 MHz, DMSO d₆) δ (ppm)=0.88/1.08 (t, 3H), 2.38-2.47 (m, 2H), 3.72 (br. s., 2H), 5.17/5.27 (s, 2H), 6.97/7.04 (s, 1H), 7.16/7.26 (d, 2H), 7.74-7.82 (m, 2H).

Intermediate 19C

### 4-[(4-amino-3-isopropyl-1H-pyrazol-1-yl)methyl]benzonitrile and 4-[(4-amino-5-isopropyl-1H-pyrazol-1-yl)methyl]benzonitrile

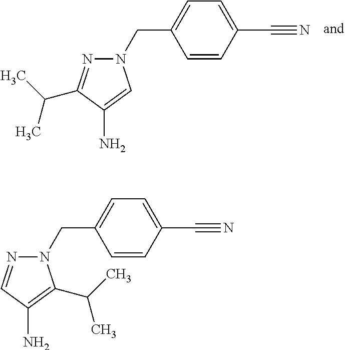 [SMILES: CC(C)c1nn(Cc2ccc(C#N)cc2)cc1N ; CC(C)c1c(N)cnn1Cc1ccc(C#N)cc1]

In analogy to intermediate 10C), in a first experiment 200 mg (0.74 mmol) and in a second experiment 1.10 g (4.1 mmol) of 4-[(3-isopropyl-4-nitro-1H-pyrazol-1-yl)methyl]benzonitrile and 4-[(5-isopropyl-4-nitro-1H-pyrazol-1-yl)methyl]benzonitrile (intermediate 21B) were reacted to give after purification of the combined crude products via a Biotage chromatography system (25 g snap KP-Sil column, hexane/0-100% ethyl acetate, then ethyl acetate/0-25% methanol) 0.66 g (57%) of the desired title compounds as a mixture.

¹H-NMR (400 MHz, DMSO d₆) δ (ppm)=1.09/1.15 (d, 6H), 2.89/3.07 (dt, 2H), 3.68 (s, 2H), 5.20/5.34 (s, 2H), 6.98/7.04 (s, 1H), 7.25 (d, 2H), 7.76-7.85 (m, 2H).

### EXAMPLES

### Example 1

### N-[1-(4-fluorobenzyl)-5-methyl-1H-pyrazol-4-yl]-2,6-dimethylquinoline-4-carboxamide

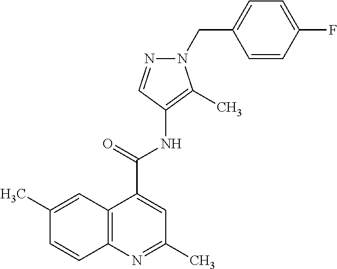 [SMILES: Cc1ccc2nc(C)cc(C(=O)Nc3cnn(Cc4ccc(F)cc4)c3C)c2c1]

To a solution of 245 mg (1.19 mmol) of a mixture of 1-(4-fluorobenzyl)-5-methyl-1H-pyrazol-4-amine and 1-(4-fluorobenzyl)-3-methyl-1H-pyrazol-4-amine (intermediate 1C) in 4.0 mL DMSO was added 453 mg (1.19 mmol) HATU, 0.26 mL N,N-diisopropylethylamine and 200 mg (0.99 mmol) commercially available 2,6-dimethylquinoline-4-carboxylic acid. The reaction mixture was stirred for 20 hours at 25° C. This mixture was directly purified via preparative HPLC (method A1) to obtain 92 mg (23%) of the desired title compound together with 208 mg (51%) of the regioisomer N-[1-(4-fluorobenzyl)-3-methyl-1H-pyrazol-4-yl]-2,6-dimethylquinoline-4-carboxamide.

1H NMR (500 MHz, DMSO d₆): δ (ppm)=2.22 (s, 3H), 2.48 (s, 3H), 2.68 (s, 3H), 5.31 (s, 2H), 7.16-7.26 (m, 4H), 7.53 (s, 1H), 7.60 (dd, 1H), 7.79 (s, 1H), 7.85 (s, 1H), 7.89 (d, 1H), 10.12 (s, 1H).

### Example 2

### 6,7-difluoro-N-[1-(4-fluorobenzyl)-5-methyl-1H-pyrazol-4-yl]-2-(trifluoromethyl)quinoline-4-carboxamide

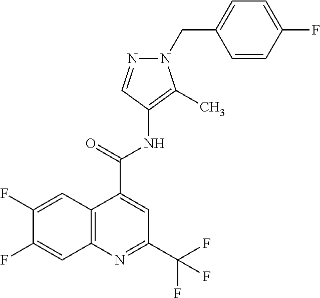 [SMILES: Cc1c(NC(=O)c2cc(C(F)(F)F)nc3cc(F)c(F)cc23)cnn1Cc1ccc(F)cc1]

In analogy to example 1), 222 mg (1.08 mmol) of a mixture of 1-(4-fluorobenzyl)-5-methyl-1H-pyrazol-4-amine and 1-(4-fluorobenzyl)-3-methyl-1H-pyrazol-4-amine (intermediate 1C) and 250 mg (0.90 mmol) 6,7-difluoro-2-(trifluoromethyl)quinoline-4-carboxylic acid (intermediate 5A) were reacted to give after purification via HPLC (method B1) 72 mg (17%) of the desired title compound together with 137 mg (32%) of the regioisomer 6,7-difluoro-N-[1-(4-fluorobenzyl)-3-methyl-1H-pyrazol-4-yl]-2-(trifluoromethyl)quinoline-4-carboxamide.

1H NMR (400 MHz, DMSO d₆): δ (ppm)=2.22 (s, 3H), 5.31 (s, 2H), 7.14-7.24 (m, 4H), 7.81 (s, 1H), 8.22-8.29 (m, 2H), 8.38 (dd, 1H), 10.37 (s, 1H).

### Example 3

### N-[1-(4-fluorobenzyl)-5-methyl-1H-pyrazol-4-yl]-2-methoxyquinoline-4-carboxamide

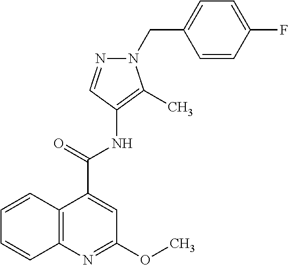 [SMILES: COc1cc(C(=O)Nc2cnn(Cc3ccc(F)cc3)c2C)c2ccccc2n1]

In analogy to example 1), 303 mg (1.48 mmol) of a mixture of 1-(4-fluorobenzyl)-5-methyl-1H-pyrazol-4-amine and 1-(4-fluorobenzyl)-3-methyl-1H-pyrazol-4-amine (intermediate 1C) and 250 mg (1.23 mmol) commercially available 2-methoxyquinoline-4-carboxylic acid were reacted to give after purification via HPLC (method C1) 97 mg (19%) of the desired title compound together with 201 mg (37%) of the regioisomer N-[1-(4-fluorobenzyl)-3-methyl-1H-pyrazol-4-yl]-2-methoxyquinoline-4-carboxamide.

1H NMR (500 MHz, DMSO d₆): δ (ppm)=2.20 (s, 3H), 4.02 (s, 3H), 5.30 (s, 2H), 7.16-7.23 (m, 5H), 7.48 (ddd, 1H), 7.71 (ddd, 1H), 7.77 (s, 1H), 7.84 (dd, 1H), 8.01 (dd, 1H), 10.13 (s, 1H).

### Example 4

### 6-bromo-N-[1-(4-fluorobenzyl)-5-methyl-1H-pyrazol-4-yl]-2-(trifluoromethyl)quinoline-4-carboxamide

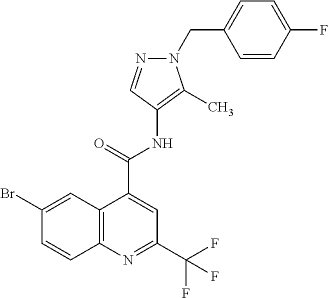 [SMILES: Cc1c(NC(=O)c2cc(C(F)(F)F)nc3ccc(Br)cc23)cnn1Cc1ccc(F)cc1]

In analogy to example 1), 192 mg (0.94 mmol) of a mixture of 1-(4-fluorobenzyl)-5-methyl-1H-pyrazol-4-amine and 1-(4-fluorobenzyl)-3-methyl-1H-pyrazol-4-amine (intermediate 1C) and 250 mg (0.78 mmol) 6-bromo-2-(trifluoromethyl)quinoline-4-carboxylic acid (intermediate 1A) were reacted to give after purification via HPLC (method A2) 30 mg (7.2%) of the desired title compound together with 99 mg (24%) of the regioisomer 6-bromo-N-[1-(4-fluorobenzyl)-3-methyl-1H-pyrazol-4-yl]-2-(trifluoromethyl)quinoline-4-carboxamide.

1H NMR (500 MHz, DMSO d₆): δ (ppm)=2.23 (s, 3H), 5.32 (s, 2H), 7.15-7.24 (m, 4H), 7.81 (s, 1H), 8.13 (dd, 1H), 8.21 (d, 1H), 8.25 (s, 1H), 8.46 (d, 1H), 10.38 (s, 1H).

### Example 5

### N⁴-[1-(4-fluorobenzyl)-5-methyl-1H-pyrazol-4-yl]quinoline-2,4-dicarboxamide

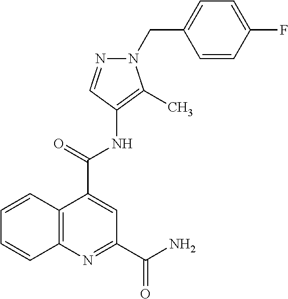 [SMILES: Cc1c(NC(=O)c2cc(C(N)=O)nc3ccccc23)cnn1Cc1ccc(F)cc1]

In analogy to example 1), 228 mg (1.11 mmol) of a mixture of 1-(4-fluorobenzyl)-5-methyl-1H-pyrazol-4-amine and 1-(4-fluorobenzyl)-3-methyl-1H-pyrazol-4-amine (intermediate 1C) and 200 mg (0.93 mmol) 2-carbamoylquinoline-4-carboxylic acid (intermediate 3A) were reacted to give after purification via HPLC (method D) 62 mg (16%) of the desired title compound together with 100 mg (25%) of the regioisomer N⁴-[1-(4-fluorobenzyl)-3-methyl-1H-pyrazol-4-yl]quinoline-2,4-dicarboxamide.

1H NMR (500 MHz, DMSO d₆): δ (ppm)=2.22 (s, 3H), 5.31 (s, 2H), 7.15-7.25 (m, 4H), 7.75-7.80 (m, 2H), 7.87 (d, 1H), 7.92 (ddd, 1H), 8.19 (d, 1H), 8.21-8.25 (m, 2H), 8.36 (d, 1H), 10.28 (s, 1H).

### Example 6

### 2-cyclopropyl-6-fluoro-N-[1-(4-fluorobenzyl)-5-methyl-1H-pyrazol-4-yl]quinoline-4-carboxamide

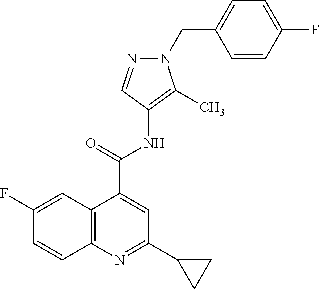 [SMILES: Cc1c(NC(=O)c2cc(C3CC3)nc3ccc(F)cc23)cnn1Cc1ccc(F)cc1]

In analogy to example 1), 213 mg (1.04 mmol) of a mixture of 1-(4-fluorobenzyl)-5-methyl-1H-pyrazol-4-amine and 1-(4-fluorobenzyl)-3-methyl-1H-pyrazol-4-amine (intermediate 1C) and 200 mg (0.87 mmol) 2-cyclopropyl-6-fluoroquinoline-4-carboxylic acid (intermediate 6A) were reacted to give after purification via HPLC (method A3) 107 mg (28%) of the desired title compound together with 172 mg (45%) of the regioisomer 2-cyclopropyl-6-fluoro-N-[1-(4-fluorobenzyl)-3-methyl-1H-pyrazol-4-yl]quinoline-4-carboxamide.

1H NMR (500 MHz, DMSO d₆): δ (ppm)=1.08-1.14 (m, 4H), 2.21 (s, 3H), 2.33-2.38 (m, 1H), 5.31 (s, 2H), 7.15-7.24 (m, 4H), 7.62-7.68 (m, 2H), 7.76-7.80 (m, 2H), 7.97 (dd, 1H), 10.16 (s, 1H).

### Example 7

### 6,8-dichloro-N-[1-(4-fluorobenzyl)-5-methyl-1H-pyrazol-4-yl]-2-(trifluoromethyl)quinoline-4-carboxamide

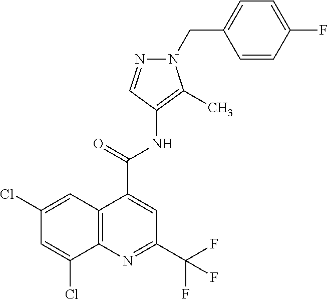 [SMILES: Cc1c(NC(=O)c2cc(C(F)(F)F)nc3c(Cl)cc(Cl)cc23)cnn1Cc1ccc(F)cc1]

In analogy to example 1), 199 mg (0.97 mmol) of a mixture of 1-(4-fluorobenzyl)-5-methyl-1H-pyrazol-4-amine and 1-(4-fluorobenzyl)-3-methyl-1H-pyrazol-4-amine (intermediate 1C) and 250 mg (0.81 mmol) 6,8-dichloro-2-(trifluoromethyl)quinoline-4-carboxylic acid (intermediate 4A) were reacted to give after purification via HPLC (method C2) 46 mg (11%) of the desired title compound together with 79 mg (19%) of the regioisomer 6,8-dichloro-N-[1-(4-fluorobenzyl)-3-methyl-1H-pyrazol-4-yl]-2-(trifluoromethyl)quinoline-4-carboxamide.

1H NMR (400 MHz, DMSO d₆): δ (ppm)=2.23 (s, 3H), 5.32 (s, 2H), 7.11-7.26 (m, 4H), 7.82 (s, 1H), 8.28 (d, 1H), 8.36-8.40 (m, 2H), 10.41 (s, 1H).

### Example 8

### 6-bromo-N-[1-(4-cyanobenzyl)-5-methyl-1H-pyrazol-4-yl]-2-(trifluoromethyl)quinoline-4-carboxamide

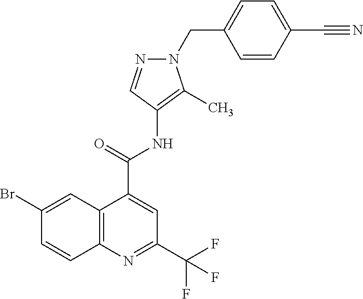 [SMILES: Cc1c(NC(=O)c2cc(C(F)(F)F)nc3ccc(Br)cc23)cnn1Cc1ccc(C#N)cc1]

In analogy to example 1), 199 mg (0.94 mmol) of a mixture of 4-[(4-amino-5-methyl-1H-pyrazol-1-yl)methyl]benzonitrile and 4-[(4-amino-3-methyl-1H-pyrazol-1-yl)methyl]benzonitrile (intermediate 4C) and 250 mg (0.78 mmol) 6-bromo-2-(trifluoromethyl)quinoline-4-carboxylic acid (intermediate 1A) were reacted to give after purification via HPLC (method C3) 66 mg (16%) of the desired title compound together with 90 mg (22%) of the regioisomer 6-bromo-N-[1-(4-cyanobenzyl)-3-methyl-1H-pyrazol-4-yl]-2-(trifluoromethyl)quinoline-4-carboxamide.

1H NMR (500 MHz, DMSO d₆): δ (ppm)=2.23 (s, 3H), 5.46 (s, 2H), 7.31 (d, 2H), 7.82-7.89 (m, 3H), 8.14 (dd, 1H), 8.22 (d, 1H), 8.27 (s, 1H), 8.49 (d, 1H), 10.43 (s, 1H).

### Example 9

### N⁴-[1-(4-cyanobenzyl)-5-methyl-1H-pyrazol-4-yl]quinoline-2,4-dicarboxamide

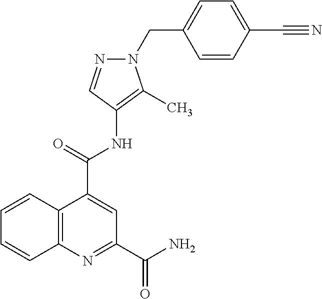 [SMILES: Cc1c(NC(=O)c2cc(C(N)=O)nc3ccccc23)cnn1Cc1ccc(C#N)cc1]

In analogy to example 1), 236 mg (1.11 mmol) of a mixture of 4-[(4-amino-5-methyl-1H-pyrazol-1-yl)methyl]benzonitrile and 4-[(4-amino-3-methyl-1H-pyrazol-1-yl)methyl]benzonitrile (intermediate 4C) and 200 mg (0.93 mmol) 2-carbamoylquinoline-4-carboxylic acid (intermediate 3A) were reacted to give after purification via HPLC (method C4) 64 mg (16%) of the desired title compound together with 73 mg (19%) of the regioisomer N⁴-[1-(4-cyanobenzyl)-5-methyl-1H-pyrazol-4-yl]quinoline-2,4-dicarboxamide.

1H NMR (400 MHz, DMSO d₆): δ (ppm)=2.22 (s, 3H), 5.45 (s, 2H), 7.30 (d, 2H), 7.78 (td, 1H), 7.81-7.89 (m, 4H), 7.90-7.95 (m, 1H), 8.18-8.26 (m, 3H), 8.36 (s, 1H), 10.32 (s, 1H).

### Example 10

### N-[1-(4-cyanobenzyl)-5-methyl-1H-pyrazol-4-yl]-2-methoxyquinoline-4-carboxamide

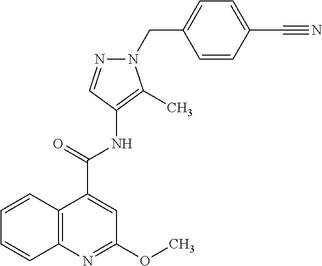 [SMILES: COc1cc(C(=O)Nc2cnn(Cc3ccc(C#N)cc3)c2C)c2ccccc2n1]

In analogy to example 1), 313 mg (1.48 mmol) of a mixture of 4-[(4-amino-5-methyl-1H-pyrazol-1-yl)methyl]benzonitrile and 4-[(4-amino-3-methyl-1H-pyrazol-1-yl)methyl]benzonitrile (intermediate 4C) and 250 mg (0.93 mmol) commercially available 2-methoxyquinoline-4-carboxylic acid were reacted to give after purification via HPLC (method C4) 87 mg (17%) of the desired title compound together with 131 mg (25%) of the regioisomer N-[1-(4-cyanobenzyl)-5-methyl-1H-pyrazol-4-yl]-2-methoxyquinoline-4-carboxamide.

1H NMR (500 MHz, DMSO d₆): δ (ppm)=2.21 (s, 3H), 4.04 (s, 3H), 5.44 (s, 2H), 7.20 (s, 1H), 7.30 (d, 2H), 7.49 (ddd, 1H), 7.72 (ddd, 1H), 7.81-7.87 (m, 4H), 8.03 (dd, 1H), 10.18 (s, 1H).

### Example 11

### 2-methoxy-N-[1-(4-methoxybenzyl)-5-methyl-1H-pyrazol-4-yl]quinoline-4-carboxamide

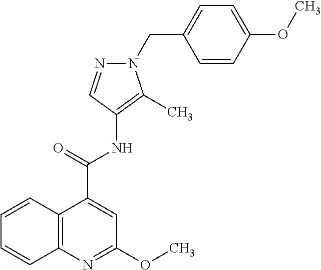 [SMILES: COc1ccc(Cn2ncc(NC(=O)c3cc(OC)nc4ccccc34)c2C)cc1]

In analogy to example 1), 321 mg (1.48 mmol) of a mixture of 1-(4-methoxybenzyl)-5-methyl-1H-pyrazol-4-amine and 1-(4-methoxybenzyl)-3-methyl-1H-pyrazol-4-amine (intermediate 6C) and 250 mg (0.93 mmol) commercially available 2-methoxyquinoline-4-carboxylic acid were reacted to give after purification via HPLC (method E1) 113 mg (22%) of the desired title compound together with 205 mg (39%) of the regioisomer 2-methoxy-N-[1-(4-methoxybenzyl)-5-methyl-1H-pyrazol-4-yl]quinoline-4-carboxamide.

1H NMR (300 MHz, DMSO d₆): δ (ppm)=2.19 (s, 3H), 3.72 (s, 3H), 4.02 (s, 3H), 5.23 (s, 2H), 6.90 (d, 2H), 7.12 (d, 2H), 7.18 (s, 1H), 7.43-7.52 (m, 1H), 7.67-7.78 (m, 2H), 7.84 (d, 1H), 8.01 (d, 1H), 10.12 (s, 1H).

### Example 12

### 6-bromo-N-[1-(4-methoxybenzyl)-5-methyl-1H-pyrazol-4-yl]-2-(trifluoromethyl)quinoline-4-carboxamide

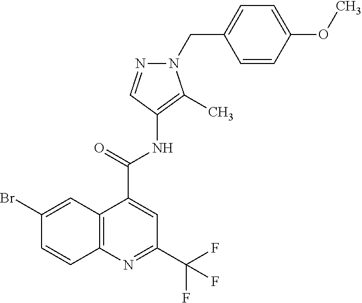 [SMILES: COc1ccc(Cn2ncc(NC(=O)c3cc(C(F)(F)F)nc4ccc(Br)cc34)c2C)cc1]

In analogy to example 1), 204 mg (0.94 mmol) of a mixture of 1-(4-methoxybenzyl)-5-methyl-1H-pyrazol-4-amine and 1-(4-methoxybenzyl)-3-methyl-1H-pyrazol-4-amine (intermediate 6C) and 250 mg (0.78 mmol) 6-bromo-2-(trifluoromethyl)quinoline-4-carboxylic acid (intermediate 1A) were reacted to give after purification via HPLC (method E1) 37 mg (8.6%) of the desired title compound together with 95 mg (22%) of the regioisomer 6-bromo-N-[1-(4-methoxybenzyl)-5-methyl-1H-pyrazol-4-yl]-2-(trifluoromethyl)quinoline-4-carboxamide.

1H NMR (400 MHz, DMSO d₆): δ (ppm)=2.24 (s, 3H), 3.75 (s, 3H), 5.27 (s, 2H), 6.91-6.95 (m, 2H), 7.15 (d, 2H), 7.80 (s, 1H), 8.15 (dd, 1H), 8.23 (d, 1H), 8.27 (s, 1H), 8.48 (d, 1H), 10.37 (s, 1H).

### Example 13

### N⁴-[1-(4-methoxybenzyl)-5-methyl-1H-pyrazol-4-yl]quinoline-2,4-dicarboxamide

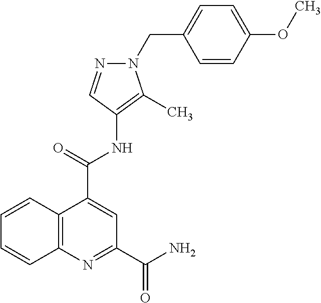 [SMILES: COc1ccc(Cn2ncc(NC(=O)c3cc(C(N)=O)nc4ccccc34)c2C)cc1]

In analogy to example 1), 241 mg (1.11 mmol) of a mixture of 1-(4-methoxybenzyl)-5-methyl-1H-pyrazol-4-amine and 1-(4-methoxybenzyl)-3-methyl-1H-pyrazol-4-amine (intermediate 6C) and 200 mg (0.93 mmol) 2-carbamoylquinoline-4-carboxylic acid (intermediate 3A) were reacted to give after purification via HPLC (method E2) 65 mg (16%) of the desired title compound together with 99 mg (25%) of the regioisomer N⁴-[1-(4-methoxybenzyl)-5-methyl-1H-pyrazol-4-yl]quinoline-2,4-dicarboxamide.

1H NMR (400 MHz, DMSO d₆): δ (ppm)=2.21 (s, 3H), 3.72 (s, 3H), 5.24 (s, 2H), 6.87-6.94 (m, 2H), 7.14 (d, 2H), 7.74-7.82 (m, 2H), 7.86 (br. s., 1H), 7.92 (ddd, 1H), 8.15-8.24 (m, 3H), 8.35 (s, 1H), 10.26 (s, 1H).

### Example 14

### 6-bromo-N-[1-(2-cyanobenzyl)-5-methyl-1H-pyrazol-4-yl]-2-(trifluoromethyl)quinoline-4-carboxamide

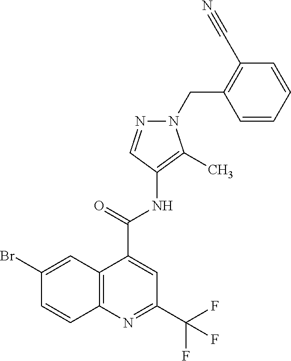 [SMILES: Cc1c(NC(=O)c2cc(C(F)(F)F)nc3ccc(Br)cc23)cnn1Cc1ccccc1C#N]

In analogy to example 1), 199 mg (0.94 mmol) of a mixture of 2-[(4-amino-5-methyl-1H-pyrazol-1-yl)methyl]benzonitrile and 2-[(4-amino-3-methyl-1H-pyrazol-1-yl)methyl]benzonitrile (intermediate 5C) and 250 mg (0.78 mmol) 6-bromo-2-(trifluoromethyl)quinoline-4-carboxylic acid (intermediate 1A) were reacted to give after purification via HPLC (method E1) 22 mg (5.2%) of the desired title compound together with 124 mg (30%) of the regioisomer 6-bromo-N-[1-(2-cyanobenzyl)-5-methyl-1H-pyrazol-4-yl]-2-(trifluoromethyl)quinoline-4-carboxamide.

1H NMR (400 MHz, DMSO d₆): δ (ppm)=2.31 (s, 3H), 5.50 (s, 2H), 7.09 (d, 1H), 7.50-7.55 (m, 1H), 7.67-7.73 (m, 1H), 7.86 (s, 1H), 7.89 (dd, 1H), 8.13 (dd, 1H), 8.22 (d, 1H), 8.27 (s, 1H), 8.48 (d, 1H), 10.43 (s, 1H).

### Example 15

### N⁴-[1-(2-cyanobenzyl)-5-methyl-1H-pyrazol-4-yl]quinoline-2,4-dicarboxamide

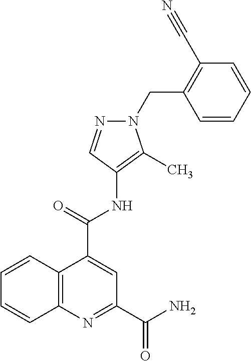 [SMILES: Cc1c(NC(=O)c2cc(C(N)=O)nc3ccccc23)cnn1Cc1ccccc1C#N]

In analogy to example 1), 236 mg (1.11 mmol) of a mixture of 2-[(4-amino-5-methyl-1H-pyrazol-1-yl)methyl]benzonitrile and 2-[(4-amino-3-methyl-1H-pyrazol-1-yl)methyl]benzonitrile (intermediate 5C) and 200 mg (0.93 mmol) 2-carbamoylquinoline-4-carboxylic acid (intermediate 3A) were reacted to give after purification via HPLC (method E1) 3.8 mg (0.92%) of the desired title compound together with 122 mg (29%) of the regioisomer N⁴-[1-(2-cyanobenzyl)-5-methyl-1H-pyrazol-4-yl]quinoline-2,4-dicarboxamide.

1H NMR (400 MHz, DMSO d₆): δ (ppm)=2.31 (s, 3H), 5.52 (s, 2H), 7.11 (d, 1H), 7.54 (td, 1H), 7.71 (td, 1H), 7.78-7.84 (m, 1H), 7.85 (s, 1H), 7.88-7.98 (m, 3H), 8.22 (d, 1H), 8.24-8.28 (m, 2H), 8.39 (s, 1H), 10.37 (s, 1H).

### Example 16

### methyl 4-({[6-bromo-2-(trifluoromethyl)quinolin-4-yl]carbonyl}amino)-1-(4-cyanobenzyl)-1H-pyrazole-5-carboxylate

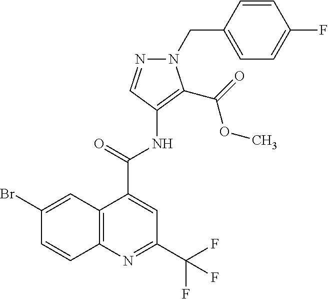 [SMILES: COC(=O)c1c(NC(=O)c2cc(C(F)(F)F)nc3ccc(Br)cc23)cnn1Cc1ccc(F)cc1]

To a solution of 300 mg (1.20 mmol) of of methyl 4-amino-1-(4-fluorobenzyl)-1H-pyrazole-5-carboxylate (intermediate 10C) in 7.2 mL DMSO was added 458 mg (1.20 mmol) HATU, 0.26 mL N,N-diisopropylethylamine and 321 mg (1.00 mmol) 6-bromo-2-(trifluoromethyl)quinoline-4-carboxylic acid (intermediate 1A). The reaction mixture was stirred for 2 hours at 25° C. Then the reaction mixture was partitioned between ethyl acetate and water. The layers were separated and the aqueous layer was extracted with ethyl acetate. The combined organic layers were washed with concentrated aqueous sodium bicarbonate and brine, dried over sodium sulfate, filtrated and evaporated to obtain a residue which was purified via a Biotage chromatography system (25 g snap KP-Sil column, hexane/0-100% ethyl acetate, then ethyl acetate/0-100% methanol) to obtain not a pure compound. This solid material was solved in methyl tert.butyl ether and stirred for 1 hour at 25° C. Then the solid was obtained via filtration and dried to obtain 300 mg (53%) of the desired title compound.

1H NMR (300 MHz, DMSO d₆): δ (ppm)=3.82 (s, 3H), 5.67 (s, 2H), 7.12-7.31 (m, 4H), 8.11-8.27 (m, 4H), 8.54 (d, 1H), 10.43 (s, 1H).

### Example 17

### 4-({[6-bromo-2-(trifluoromethyl)quinolin-4-yl]carbonyl}amino)-1-(4-fluorobenzyl)-1H-pyrazole-5-carboxylic acid

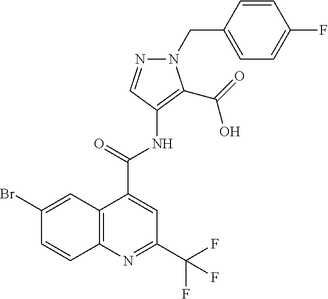 [SMILES: O=C(Nc1cnn(Cc2ccc(F)cc2)c1C(=O)O)c1cc(C(F)(F)F)nc2ccc(Br)cc12]

To a solution of 300 mg (0.54 mmol) of the compound from example 16) in 4.8 mL methanol and 5 ml THF was added a solution of 392 mg sodium hydroxide in 9.6 mL water. This mixture was stirred for 2 hours at 40° C. and then concentrated in vacuum. The residue was diluted with water and 10% aq. sulfuric acid was added up to pH 4. After stirring for additional 15 minutes the formed solid was isolated by filtration and dried in vacuum. Using this methodology we got the desired title compound. Yield: 260 mg (86%)

1H NMR (400 MHz, DMSO d₆): δ (ppm)=5.72 (s, 2H), 7.12-7.19 (m, 2H), 7.20-7.27 (m, 2H), 8.13 (dd, 1H), 8.19 (d, 2H), 8.21-8.25 (m, 2H), 8.58 (d, 1H), 10.58 (s, 1H).

### Example 18

### 6-bromo-N-[5-carbamoyl-1-(4-fluorobenzyl)-1H-pyrazol-4-yl]-2-(trifluoromethyl)quinoline-4-carboxamide

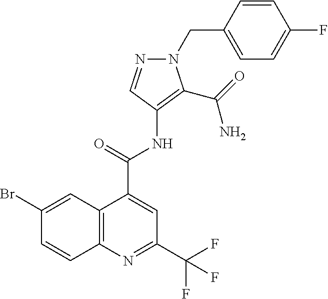 [SMILES: NC(=O)c1c(NC(=O)c2cc(C(F)(F)F)nc3ccc(Br)cc23)cnn1Cc1ccc(F)cc1]

To a solution of 130 mg (0.24 mmol) of the compound from example 17) in 1.3 mL DMSO was added 110 mg (0.29 mmol) HATU, 63 μL N,N-diisopropylethylamine and 726 μL (0.36 mmol) 0.5M solution of ammonia in dioxan. The reaction mixture was stirred for 20 hours at 25° C. An additional amount of HATU and ammonia was added and stirring was continued for several hours at 25° C. Then reaction mixture was evaporated to dryness and the obtained residue was purified via preparative HPLC (method G) to obtain 18 mg (13%) of the desired title compound.

1H NMR (400 MHz, DMSO d₆): δ (ppm)=5.54 (s, 2H), 7.15-7.20 (m, 2H), 7.24-7.29 (m, 2H), 7.89 (s, 3H), 8.12 (dd, 1H), 8.20 (d, 1H), 8.33 (s, 1H), 8.60 (d, 1H), 10.58 (s, 1H).

### Example 19

### methyl 4-({[6-bromo-2-(trifluoromethyl)quinolin-4-yl]carbonyl}amino)-1-(4-cyanobenzyl)-1H-pyrazole-5-carboxylate

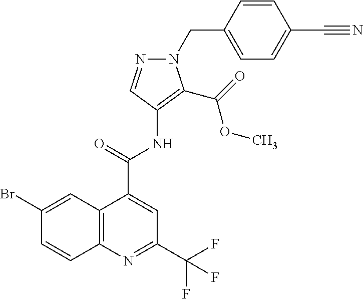 [SMILES: COC(=O)c1c(NC(=O)c2cc(C(F)(F)F)nc3ccc(Br)cc23)cnn1Cc1ccc(C#N)cc1]

To a solution of 270 mg (1.05 mmol) of of methyl 4-amino-1-(4-cyanobenzyl)-1H-pyrazole-5-carboxylate (intermediate 11C) in 6.3 mL DMSO was added 401 mg (1.05 mmol) HATU, 0.23 mL N,N-diisopropylethylamine and 281 mg (0.88 mmol) 6-bromo-2-(trifluoromethyl)quinoline-4-carboxylic acid (intermediate 1A). The reaction mixture was stirred for 1 hour at 25° C. Then the reaction mixture was partitioned between ethyl acetate and water. The layers were separated and the aqueous layer was extracted with ethyl acetate. The combined organic layers were washed with concentrated aqueous sodium hydrogencarbonate and brine, dried over sodium sulfate, filtrated and evaporated to obtain a residue which was stirred solved in ethyl acetate for 2 hours at 25° C. Then the solid was obtained via filtration and dried to obtain 290 mg (57%) of the desired title compound.

1H NMR (400 MHz, DMSO d₆): δ (ppm)=3.79 (s, 3H), 5.79 (s, 2H), 7.32 (d, 2H), 7.83 (d, 2H), 8.15 (dd, 1H), 8.20-8.25 (m, 3H), 8.55 (d, 1H), 10.42 (s, 1H).

### Example 20

### 4-({[6-bromo-2-(trifluoromethyl)quinolin-4-yl]carbonyl}amino)-1-(4-cyanobenzyl)-1H-pyrazole-5-carboxylic acid

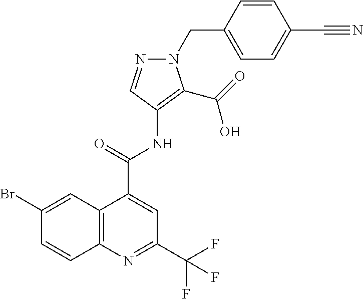 [SMILES: N#Cc1ccc(Cn2ncc(NC(=O)c3cc(C(F)(F)F)nc4ccc(Br)cc34)c2C(=O)O)cc1]

In analogy to example 17), 290 mg (0.52 mmol) of the compound from example 19) were reacted to give 270 mg (89%) of the desired title compound.

1H NMR (400 MHz, DMSO d₆): δ (ppm)=5.82 (s, 2H), 7.29 (d, 2H), 7.82 (d, 2H), 8.14 (dd, 1H), 8.20-8.27 (m, 3H), 8.57 (d, 1H), 10.39 (s, 1H).

### Example 21

### 6-bromo-N-[1-(4-cyanobenzyl)-5-(methylcarbamoyl)-1H-pyrazol-4-yl]-2-(trifluoromethyl)quinoline-4-carboxamide

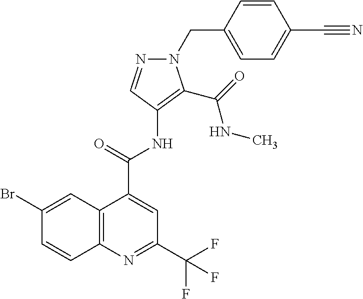 [SMILES: CNC(=O)c1c(NC(=O)c2cc(C(F)(F)F)nc3ccc(Br)cc23)cnn1Cc1ccc(C#N)cc1]

In analogy to example 18), 135 mg (0.25 mmol) of the compound from example 20) and 186 μL (0.37 mmol) of 2.0M methylamine solution in THF were reacted to give after purification via HPLC (method G) 4.7 mg (3.1%) of the desired title compound.

1H NMR (400 MHz, DMSO d₆): δ (ppm)=2.73 (d, 3H), 5.63 (s, 2H), 7.36 (d, 2H), 7.84 (d, 2H), 7.94 (s, 1H), 8.15 (dd, 1H), 8.22 (d, 1H), 8.33 (s, 1H), 8.43 (s, 1H), 8.62 (s, 1H), 10.67 (s, 1H).

### Example 22

### 6-bromo-N-[5-carbamoyl-1-(4-cyanobenzyl)-1H-pyrazol-4-yl]-2-(trifluoromethyl)quinoline-4-carboxamide

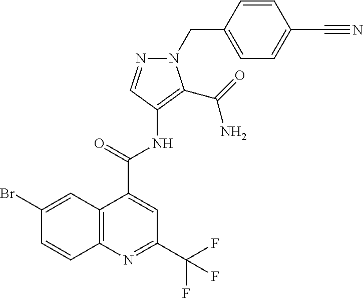 [SMILES: N#Cc1ccc(Cn2ncc(NC(=O)c3cc(C(F)(F)F)nc4ccc(Br)cc34)c2C(N)=O)cc1]

In analogy to example 18), 135 mg (0.25 mmol) of the compound from example 20) and 744 μL (0.37 mmol) of 0.5M ammonia solution in dioxan were reacted to give after two subsequent purifications via HPLC (method G) 14 mg (10%) of the desired title compound.

1H NMR (400 MHz, DMSO d₆): δ (ppm)=5.69 (s, 2H), 7.35 (d, 2H), 7.78-8.03 (m, 5H), 8.15 (dd, 1H), 8.23 (d, 1H), 8.36 (s, 1H), 8.64 (s, 1H), 10.62 (s, 1H).

### Example 23

### 6-bromo-N-[1-(4-cyanobenzyl)-5-methyl-1H-pyrazol-4-yl]-2-cyclopropylquinoline-4-carboxamide

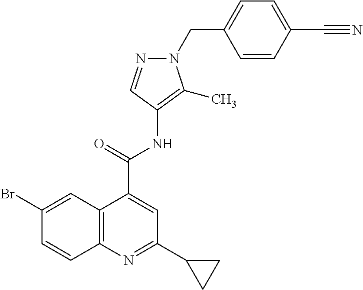 [SMILES: Cc1c(NC(=O)c2cc(C3CC3)nc3ccc(Br)cc23)cnn1Cc1ccc(C#N)cc1]

In analogy to example 1), 218 mg (1.03 mmol) of a mixture of 4-[(4-amino-5-methyl-1H-pyrazol-1-yl)methyl]benzonitrile and and 4-[(4-amino-3-methyl-1H-pyrazol-1-yl)methyl]benzonitrile (intermediate 4C) and 250 mg (0.86 mmol) commercially available 6-bromo-2-cyclopropylquinoline-4-carboxylic acid were reacted to give after purification via HPLC (method B2) 95 mg (22%) of the desired title compound together with 143 mg (33%) of the regioisomer 6-bromo-N-[1-(4-cyanobenzyl)-3-methyl-1H-pyrazol-4-yl]-2-cyclopropylquinoline-4-carboxamide.

1H NMR (400 MHz, DMSO d₆): δ (ppm)=1.10-1.20 (m, 4H), 2.22 (s, 3H), 2.35-2.42 (m, 1H), 5.46 (s, 2H), 7.30 (d, 2H), 7.70 (s, 1H), 7.82-7.90 (m, 5H), 8.27 (t, 1H), 10.26 (s, 1H).

### Example 24

### N⁴-[1-(4-cyanobenzyl)-5-methyl-1H-pyrazol-4-yl]-7-fluoroquinoline-2,4-dicarboxamide

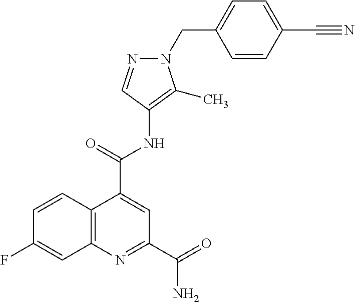 [SMILES: Cc1c(NC(=O)c2cc(C(N)=O)nc3cc(F)ccc23)cnn1Cc1ccc(C#N)cc1]

In analogy to example 1), 272 mg (1.28 mmol) of a mixture of 4-[(4-amino-5-methyl-1H-pyrazol-1-yl)methyl]benzonitrile and 4-[(4-amino-3-methyl-1H-pyrazol-1-yl)methyl]benzonitrile (intermediate 4C) and 250 mg (1.07 mmol) 2-carbamoyl-7-fluoroquinoline-4-carboxylic acid (intermediate 2A) were reacted to give after purification via HPLC (method B3) 84 mg (17%) of the desired title compound together with 74 mg (16%) of the regioisomer N⁴-[1-(4-cyanobenzyl)-3-methyl-1H-pyrazol-4-yl]-7-fluoroquinoline-2,4-dicarboxamide.

1H NMR (400 MHz, DMSO d₆): δ (ppm)=2.21 (s, 3H), 5.44 (s, 2H), 7.30 (d, 2H), 7.73 (td, 1H), 7.81-7.93 (m, 5H), 8.24 (s, 1H), 8.30-8.38 (m, 2H), 10.36 (s, 1H).

### Example 25

### 6-chloro-N⁴-[1-(4-cyanobenzyl)-5-methyl-1H-pyrazol-4-yl]-7-fluoroquinoline-2,4-dicarboxamide

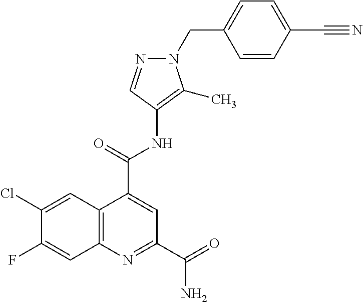 [SMILES: Cc1c(NC(=O)c2cc(C(N)=O)nc3cc(F)c(Cl)cc23)cnn1Cc1ccc(C#N)cc1]

In analogy to example 1), 237 mg (1.18 mmol) of a mixture of 4-[(4-amino-5-methyl-1H-pyrazol-1-yl)methyl]benzonitrile and 4-[(4-amino-3-methyl-1H-pyrazol-1-yl)methyl]benzonitrile (intermediate 4C) and 250 mg (0.93 mmol) 2-carbamoyl-6-chloro-7-fluoroquinoline-4-carboxylic acid (intermediate 7A) were reacted to give after purification via HPLC (method B4) 42 mg (9%) of the desired title compound together with 63 mg (13%) of the regioisomer 6-chloro-N⁴-[1-(4-cyanobenzyl)-3-methyl-1H-pyrazol-4-yl]-7-fluoroquinoline-2,4-dicarboxamide.

1H NMR (400 MHz, DMSO d₆): δ (ppm)=2.22 (s, 3H), 5.45 (s, 2H), 7.30 (d, 2H), 7.81-7.86 (m, 3H), 7.96 (s, 1H), 8.11 (d, 1H), 8.34 (s, 1H), 8.36 (s, 1H), 8.51 (d, 1H), 10.43 (s, 1H).

### Example 26

### N⁴-[1-(3,4-difluorobenzyl)-5-methyl-1H-pyrazol-4-yl]quinoline-2,4-dicarboxamide

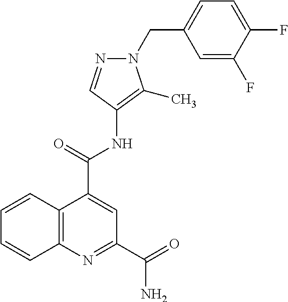 [SMILES: Cc1c(NC(=O)c2cc(C(N)=O)nc3ccccc23)cnn1Cc1ccc(F)c(F)c1]

In analogy to example 1), 250 mg (1.12 mmol) of a mixture of 1-(3,4-difluorobenzyl)-5-methyl-1H-pyrazol-4-amine and 1-(3,4-difluorobenzyl)-3-methyl-1H-pyrazol-4-amine (intermediate 3C) and 202 mg (0.93 mmol) 2-carbamoylquinoline-4-carboxylic acid (intermediate 3A) were reacted to give after purification via HPLC (method H1) 45 mg (11%) of the desired title compound together with 99 mg (24%) of the regioisomer N⁴-[1-(3,4-difluorobenzyl)-3-methyl-1H-pyrazol-4-yl]quinoline-2,4-dicarboxamide.

1H NMR (400 MHz, DMSO d₆): δ (ppm)=2.19 (s, 3H), 5.28 (s, 2H), 7.14-7.21 (m, 1H), 7.36-7.49 (m, 2H), 7.79 (ddd, 1H), 7.87-7.98 (m, 2H), 8.18-8.24 (m, 3H), 8.30 (s, 1H), 8.39 (d, 1H), 10.42 (s, 1H).

### Example 27

### N⁴-[1-(3-fluorobenzyl)-5-methyl-1H-pyrazol-4-yl]quinoline-2,4-dicarboxamide

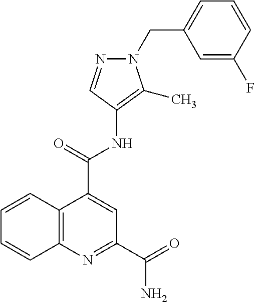 [SMILES: Cc1c(NC(=O)c2cc(C(N)=O)nc3ccccc23)cnn1Cc1cccc(F)c1]

In analogy to example 1), 375 mg (1.12 mmol) of a mixture of 1-(3-fluorobenzyl)-5-methyl-1H-pyrazol-4-amine and 1-(3-fluorobenzyl)-3-methyl-1H-pyrazol-4-amine (intermediate 2C) and 329 mg (1.52 mmol) 2-carbamoylquinoline-4-carboxylic acid (intermediate 3A) were reacted to give after purification via HPLC (method H2) 30 mg (4.7%) of the desired title compound together with 131 mg (20%) of the regioisomer N⁴-[1-(3-fluorobenzyl)-3-methyl-1H-pyrazol-4-yl]quinoline-2,4-dicarboxamide.

1H NMR (400 MHz, DMSO d₆): δ (ppm)=2.22 (s, 3H), 5.36 (s, 2H), 6.92-7.02 (m, 2H), 7.09-7.15 (m, 1H), 7.40 (td, 1H), 7.75-7.84 (m, 2H), 7.86 (br. s., 1H), 7.92 (ddd, 1H), 8.18-8.27 (m, 3H), 8.36 (s, 1H), 10.30 (s, 1H).

### Example 28

### N⁴-[1-(cyclohexylmethyl)-5-methyl-1H-pyrazol-4-yl]quinoline-2,4-dicarboxamide

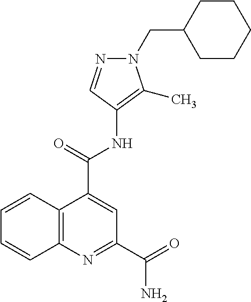 [SMILES: Cc1c(NC(=O)c2cc(C(N)=O)nc3ccccc23)cnn1CC1CCCCC1]

In analogy to example 1), 425 mg (2.20 mmol) of a mixture of 1-(cyclohexylmethyl)-5-methyl-1H-pyrazol-4-amine and 1-(cyclohexylmethyl)-3-methyl-1H-pyrazol-4-amine (intermediate 9C) and 396 mg (1.83 mmol) 2-carbamoylquinoline-4-carboxylic acid (intermediate 3A) were reacted to give after purification via HPLC (method I) 49 mg (6.4%) of the desired title compound together with 81 mg (11%) of the regioisomer N⁴-[1-(cyclohexylmethyl)-3-methyl-1H-pyrazol-4-yl]quinoline-2,4-dicarboxamide.

1H NMR (400 MHz, DMSO d₆): δ (ppm)=0.93-1.05 (m, 2H), 1.11-1.25 (m, 3H), 1.47-1.72 (m, 5H), 1.74-1.86 (m, 1H), 2.25 (s, 3H), 3.87 (d, 2H), 7.70 (s, 1H), 7.78 (ddd, 1H), 7.87 (d, 1H), 7.92 (ddd, 1H), 8.18-8.25 (m, 3H), 8.36 (d, 1H), 10.22 (s, 1H).

### Example 29

### N⁴-[5-methyl-1-(pyridin-4-ylmethyl)-1H-pyrazol-4-yl]quinoline-2,4-dicarboxamide

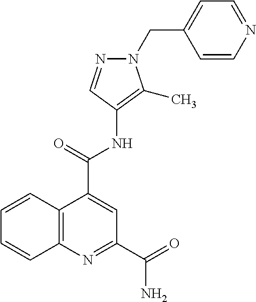 [SMILES: Cc1c(NC(=O)c2cc(C(N)=O)nc3ccccc23)cnn1Cc1ccncc1]

In analogy to example 1), 141 mg (0.758 mmol) of a mixture of 5-methyl-1-(pyridin-4-ylmethyl)-1H-pyrazol-4-amine and 3-methyl-1-(pyridin-4-ylmethyl)-1H-pyrazol-4-amine (intermediate 8C) and 135 mg (0.62 mmol) 2-carbamoylquinoline-4-carboxylic acid (intermediate 3A) were reacted to give after purification via HPLC (method J) 24 mg (8.5%) of the desired title compound together with 43 mg (16%) of the regioisomer N⁴-[3-methyl-1-(pyridin-4-ylmethyl)-1H-pyrazol-4-yl]quinoline-2,4-dicarboxamide.

1H NMR (400 MHz, DMSO d₆): δ (ppm)=2.21 (s, 3H), 5.40 (s, 2H), 7.07 (d, 2H), 7.75-7.81 (m, 1H), 7.84 (s, 1H), 7.86-7.88 (m, 1H), 7.89-7.97 (m, 1H), 8.18-8.27 (m, 3H), 8.36 (br. s., 1H), 8.51-8.56 (m, 2H), 10.34 (s, 1H). 2.22 (s, 3H), 5.40 (s, 2H), 7.07 (d, 2H), 7.76-7.81 (m, 1H), 7.84 (s, 1H), 7.85-7.88 (m, 1H), 7.89-7.97 (m, 1H), 8.18-8.27 (m, 3H), 8.36 (s, 1H), 8.51-8.56 (m, 2H), 10.34 (s, 1H).

### Example 30

### N⁴-[1-(4-cyanobenzyl)-5-ethyl-1H-pyrazol-4-yl]-7-fluoroquinoline-2,4-dicarboxamide

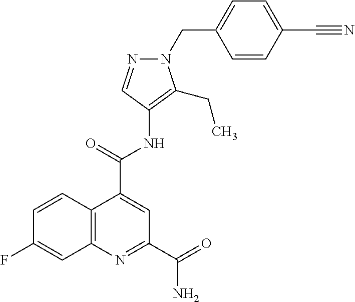 [SMILES: CCc1c(NC(=O)c2cc(C(N)=O)nc3cc(F)ccc23)cnn1Cc1ccc(C#N)cc1]

In analogy to example 1), 125 mg (0.55 mmol) of a mixture of 4-[(4-amino-5-ethyl-1H-pyrazol-1-yl)methyl]benzonitrile and 4-[(4-amino-3-ethyl-1H-pyrazol-1-yl)methyl]benzonitrile (intermediate 18C) and 155 mg (0.66 mmol) 2-carbamoyl-7-fluoroquinoline-4-carboxylic acid (intermediate 2A) were reacted to give after purification via HPLC (Chromatorex RP C-18 10 μm; 125*30 mm column, water/30-100% acetonitril) 8.8 mg (3.3%) of the desired title compound together with 58 mg (23%) of the regioisomer N⁴-[1-(4-cyanobenzyl)-3-ethyl-1H-pyrazol-4-yl]-7-fluoroquinoline-2,4-dicarboxamide.

1H NMR (400 MHz, DMSO d₆): δ (ppm)=0.99 (t, 3H), 2.71 (q, 2H), 5.48 (s, 2H), 7.31 (d, 2H), 7.77 (td, 1H), 7.84-7.87 (m, 3H), 7.92 (dd, 1H), 7.97 (s, 1H), 8.18 (s, 1H), 8.24 (s, 1H), 8.33 (dd, 1H), 8.40 (d, 1H), 10.38 (s, 1H).

### Example 31

### N⁴-[1-(4-cyanobenzyl)-5-ethyl-1H-pyrazol-4-yl]quinoline-2,4-dicarboxamide

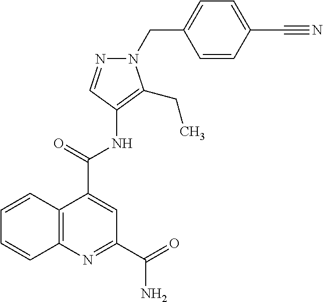 [SMILES: CCc1c(NC(=O)c2cc(C(N)=O)nc3ccccc23)cnn1Cc1ccc(C#N)cc1]

In analogy to example 1), 125 mg (0.55 mmol) of a mixture of 4-[(4-amino-5-ethyl-1H-pyrazol-1-yl)methyl]benzonitrile and 4-[(4-amino-3-ethyl-1H-pyrazol-1-yl)methyl]benzonitrile (intermediate 18C) and 143 mg (0.66 mmol) 2-carbamoylquinoline-4-carboxylic acid (intermediate 3A) were reacted to give after purification via HPLC (method K) 12.1 mg (4.4%) of the desired title compound together with 32 mg (13%) of the regioisomer N⁴-[1-(4-cyanobenzyl)-3-ethyl-1H-pyrazol-4-yl]quinoline-2,4-dicarboxamide.

1H NMR (400 MHz, DMSO d₆): δ (ppm)=1.00 (t, 3H), 2.72 (q, 2H), 5.49 (s, 2H), 7.32 (d, 2H), 7.81 (ddd, 1H), 7.84-7.88 (m, 3H), 7.90-7.97 (m, 2H), 8.20-8.25 (m, 3H), 8.37-8.45 (m, 1H), 10.34 (s, 1H).

### Example 32

### 6-chloro-N⁴-[1-(4-cyanobenzyl)-5-ethyl-1H-pyrazol-4-yl]-7-fluoroquinoline-2,4-dicarboxamide

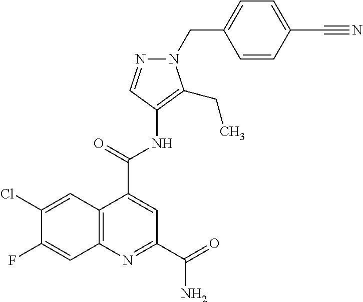 [SMILES: CCc1c(NC(=O)c2cc(C(N)=O)nc3cc(F)c(Cl)cc23)cnn1Cc1ccc(C#N)cc1]

In analogy to example 1), 125 mg (0.55 mmol) of a mixture of 4-[(4-amino-5-ethyl-1H-pyrazol-1-yl)methyl]benzonitrile and 4-[(4-amino-3-ethyl-1H-pyrazol-1-yl)methyl]benzonitrile (intermediate 18C) and 178 mg (0.66 mmol) 2-carbamoyl-6-chloro-7-fluoroquinoline-4-carboxylic acid (intermediate 7A) were reacted to give after purification via HPLC (Chromatorex RP C-18 10 μm; 125*30 mm column, water/30-100% acetonitrile) 19 mg (7.5%) of the desired title compound together with 29 mg (11%) of the regioisomer 6-chloro-N⁴-[1-(4-cyanobenzyl)-3-ethyl-1H-pyrazol-4-yl]-7-fluoroquinoline-2,4-dicarboxamide.

1H NMR (400 MHz, DMSO d₆): δ (ppm)=1.00 (t, 3H), 2.72 (q, 2H), 5.49 (s, 2H), 7.32 (d, 2H), 7.84-7.88 (m, 3H), 8.01 (s, 1H), 8.14 (d, 1H), 8.33 (s, 1H), 8.41 (s, 1H), 8.50 (d, 1H), 10.44 (s, 1H).

### Example 33

### N⁴-[1-(4-cyanobenzyl)-5-isopropyl-1H-pyrazol-4-yl]-7-fluoroquinoline-2,4-dicarboxamide

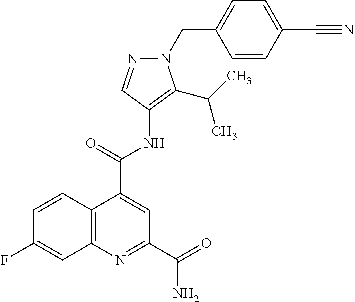 [SMILES: CC(C)c1c(NC(=O)c2cc(C(N)=O)nc3cc(F)ccc23)cnn1Cc1ccc(C#N)cc1]

In analogy to example 1), 200 mg (0.83 mmol) of a mixture of 4-[(4-amino-5-isopropyl-1H-pyrazol-1-yl)methyl]benzonitrile and 4-[(4-amino-3-isopropyl-1H-pyrazol-1-yl)methyl]benzonitrile (intermediate 19C) and 234 mg (1.0 mmol) 2-carbamoyl-7-fluoroquinoline-4-carboxylic acid (intermediate 2A) were reacted to give after purification via HPLC (Chromatorex RP C-18 10 μm; 125*30 mm column, water/30-100% acetonitrile) 15 mg (3.6%) of the desired title compound together with 135 mg (32%) of the regioisomer N⁴-[1-(4-cyanobenzyl)-3-isopropyl-1H-pyrazol-4-yl]-7-fluoroquinoline-2,4-dicarboxamide.

1H NMR (400 MHz, Acetone d₆): δ (ppm)=1.29 (d, 6H), 3.29 (dt, 1H), 5.59 (s, 2H), 7.11 (s, 1H), 7.38 (d, 2H), 7.69 (ddd, 1H), 7.78-7.83 (m, 3H), 7.85 (dd, 1H), 8.13 (s, 1H), 8.26 (s, 1H), 8.40 (s, 1H), 8.55 (dd, 1H), 9.35 (s, 1H).

### Example 34

### N⁴-{5-methyl-1-[(1-methyl-1H-pyrazol-3-yl)methyl]-1H-pyrazol-4-yl}quinoline-2,4-dicarboxamide

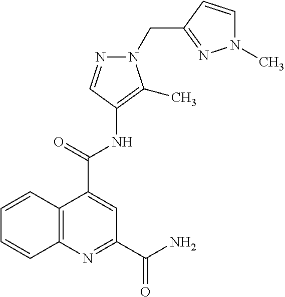 [SMILES: Cc1c(NC(=O)c2cc(C(N)=O)nc3ccccc23)cnn1Cc1ccn(C)n1]

In analogy to example 1), 211 mg (1.10 mmol) of a mixture of 5-methyl-1-[(1-methyl-1H-pyrazol-3-yl)methyl]-1H-pyrazol-4-amine and 3-methyl-1-[(1-methyl-1H-pyrazol-3-yl)methyl]-1H-pyrazol-4-amine (intermediate 12C) and 199 mg (0.92 mmol) 2-carbamoylquinoline-4-carboxylic acid (intermediate 3A) were reacted to give after purification via HPLC (method L) 6 mg (1%) of the desired title compound together with 12 mg (3%) of the regioisomer N⁴-{3-methyl-1-[(1-methyl-1H-pyrazol-3-yl)methyl]-1H-pyrazol-4-yl}quinoline-2,4-dicarboxamide.

1H NMR (400 MHz, DMSO d₆): δ (ppm)=2.29 (s, 3H), 3.80 (s, 3H), 5.20 (s, 2H), 6.06 (d, 1H), 7.61 (d, 1H), 7.70 (s, 1H), 7.79 (m, 1H), 7.87 (br.s., 1H), 7.93 (m, 1H), 8.20 (d, 1H), 8.23 (d, 1H), 8.24 (s, 1H), 8.37 (br.s., 1H), 10.25 (s, 1H).

### Example 35

### 6-bromo-N-{1-[(3-ethyl-1,2,4-oxadiazol-5-yl)methyl]-5-methyl-1H-pyrazol-4-yl}-2-(trifluoromethyl)quinoline-4-carboxamide

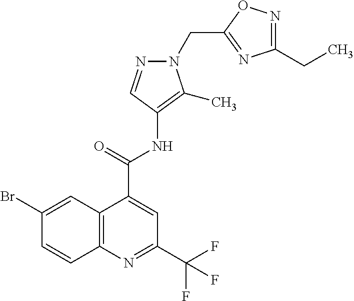 [SMILES: CCc1noc(Cn2ncc(NC(=O)c3cc(C(F)(F)F)nc4ccc(Br)cc34)c2C)n1]

In analogy to example 1), 250 mg (1.21 mmol) of a mixture of 1-[(3-ethyl-1,2,4-oxadiazol-5-yl)methyl]-5-methyl-1H-pyrazol-4-amine and 1-[(3-ethyl-1,2,4-oxadiazol-5-yl)methyl]-3-methyl-1H-pyrazol-4-amine (intermediate 14C) and 203 mg (0.60 mmol) 6-bromo-2-(trifluoromethyl)quinoline-4-carboxylic acid (intermediate 1A) were reacted to give after purification via HPLC (method 0) 62 mg (10%) of the desired title compound together with 18 mg (3%) of the regioisomer 6-bromo-N-{1-[(3-ethyl-1,2,4-oxadiazol-5-yl)methyl]-3-methyl-1H-pyrazol-4-yl}-2-(trifluoromethyl)quinoline-4-carboxamide.

1H NMR (400 MHz, DMSO d₆): δ (ppm)=1.22 (t, 3H), 2.33 (s, 3H), 2.73 (q, 2H), 5.75 (s, 2H), 7.85 (s, 1H), 8.15 (dd, 1H), 8.23 (d, 1H), 8.30 (s, 1H), 8.49 (d, 1H), 10.46 (s, 1H).

### Example 36

### N⁴-(5-methyl-1-{[5-(methylcarbamoyl)-1,2,4-oxadiazol-3-yl]methyl}-1H-pyrazol-4-yl)quinoline-2,4-dicarboxamide

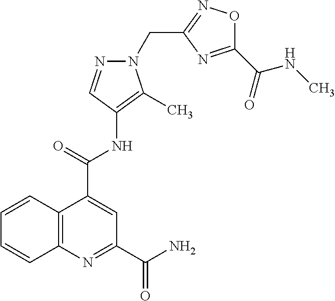 [SMILES: CNC(=O)c1nc(Cn2ncc(NC(=O)c3cc(C(N)=O)nc4ccccc34)c2C)no1]

In analogy to example 1), 140 mg (0.59 mmol) of a mixture of 3-[(4-amino-5-methyl-1H-pyrazol-1-yl)methyl]-N-methyl-1,2,4-oxadiazole-5-carboxamide and 3-[(4-amino-3-methyl-1H-pyrazol-1-yl)methyl]-N-methyl-1,2,4-oxadiazole-5-carboxamide (intermediate 15C) and 107 mg (0.49 mmol) 2-carbamoylquinoline-4-carboxylic acid (intermediate 3A) were reacted to give after purification via HPLC (method Q) 17 mg (8%) of the desired title compound together with 14 mg (7%) of the regioisomer N⁴-(3-methyl-1-{[5-(methylcarbamoyl)-1,2,4-oxadiazol-3-yl]methyl}-1H-pyrazol-4-yl)quinoline-2,4-dicarboxamide.

1H NMR (400 MHz, DMSO d₆): δ (ppm)=2.35 (s, 3H), 2.79 (d, 3H), 5.60 (s, 2H), 7.80 (m, 1H), 7.80 (s, 1H), 7.90 (br.s., 1H), 7.94 (m, 1H), 8.21 (d, 1H), 8.24 (d, 1H), 8.26 (s, 1H), 8.39 (br.s., 1H), 9.32 (br.q., 1H), 10.46 (s, 1H).

### Example 37

### N⁴-{1-[(3-ethyl-1,2,4-oxadiazol-5-yl)methyl]-5-methyl-1H-pyrazol-4-yl}quinoline-2,4-dicarboxamide

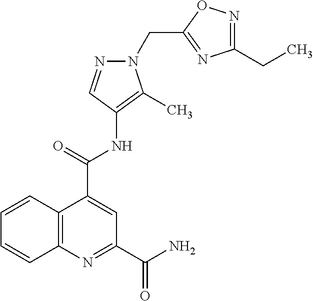 [SMILES: CCc1noc(Cn2ncc(NC(=O)c3cc(C(N)=O)nc4ccccc34)c2C)n1]

In analogy to example 1), 250 mg (1.30 mmol) of a mixture of 1-[(3-ethyl-1,2,4-oxadiazol-5-yl)methyl]-5-methyl-1H-pyrazol-4-amine and 1-[(3-ethyl-1,2,4-oxadiazol-5-yl)methyl]-3-methyl-1H-pyrazol-4-amine (intermediate 14C) and 145 mg (0.60 mmol) 2-carbamoylquinoline-4-carboxylic acid (intermediate 3A) were reacted to give after purification via HPLC (method P) 10 mg (4%) of the desired title compound together with 5 mg (2%) of the regioisomer N⁴-{1-[(3-ethyl-1,2,4-oxadiazol-5-yl)methyl]-3-methyl-1H-pyrazol-4-yl}quinoline-2,4-dicarboxamide.

1H NMR (400 MHz, DMSO d₆): δ (ppm)=1.22 (t, 3H), 2.33 (s, 3H), 2.73 (q, 2H), 5.74 (s, 2H), 7.80 (m, 1H), 7.83 (s, 1H), 7.88 (br.s., 1H), 7.94 (m, 1H), 8.21 (d, 1H), 8.26 (d, 1H), 8.28 (s, 1H), 8.38 (br.s., 1H), 10.37 (s, 1H).

### Example 38

### 6-bromo-N-(1-{[1-(ethylsulfonyl)piperidin-4-yl]methyl}-5-methyl-1H-pyrazol-4-yl)-2-(trifluoromethyl)quinoline-4-carboxamide

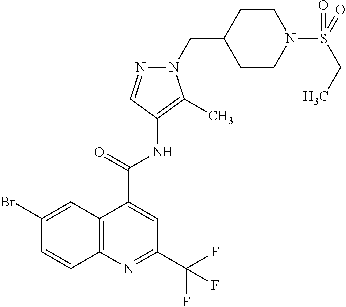 [SMILES: CCS(=O)(=O)N1CCC(Cn2ncc(NC(=O)c3cc(C(F)(F)F)nc4ccc(Br)cc34)c2C)CC1]

In analogy to example 1), 325 mg (1.13 mmol) of a mixture of 1-{[1-(ethylsulfonyl)piperidin-4-yl]methyl}-5-methyl-1H-pyrazol-4-amine and 1-{[1-(ethylsulfonyl)piperidin-4-yl]methyl}-3-methyl-1H-pyrazol-4-amine (intermediate 17C) and 159 mg (0.47 mmol) 6-bromo-2-(trifluoromethyl)quinoline-4-carboxylic acid (intermediate 1A) were reacted to give after purification via HPLC (method U) 55 mg (20%) of the desired title compound together with 70 mg (25%) of the regioisomer 6-bromo-N-(1-{[1-(ethylsulfonyl)piperidin-4-yl]methyl}-3-methyl-1H-pyrazol-4-yl)-2-(trifluoromethyl)quinoline-4-carboxamide.

1H NMR (400 MHz, DMSO d₆): δ (ppm)=1.20 (t, 3H), 1.27 (m, 2H), 1.59 (m, 2H), 1.99 (m, 1H), 2.29 (s, 3H), 2.77 (m, 2H), 3.02 (q, 2H), 3.60 (m, 2H), 3.98 (d, 2H), 7.77 (s, 1H), 8.14 (dd, 1H), 8.23 (d, 1H), 8.26 (s, 1H), 8.47 (d, 1H), 10.36 (s, 1H).

### Example 39

### N⁴-{5-methyl-1-[(2-methyl-1, 3-thiazol-4-yl)methyl]-1H-pyrazol-4-yl}quinoline-2,4-dicarboxamide

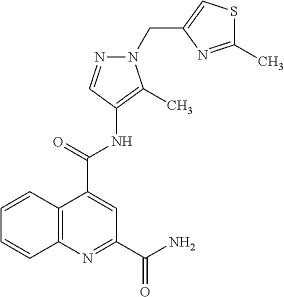 [SMILES: Cc1nc(Cn2ncc(NC(=O)c3cc(C(N)=O)nc4ccccc34)c2C)cs1]

In analogy to example 1), 225 mg (1.08 mmol) of a mixture of 5-methyl-1-[(2-methyl-1,3-thiazol-4-yl)methyl]-1H-pyrazol-4-amine and 3-methyl-1-[(2-methyl-1,3-thiazol-4-yl)methyl]-1H-pyrazol-4-amine (intermediate 16C) and 216 mg (0.90 mmol) 2-carbamoylquinoline-4-carboxylic acid (intermediate 3A) were reacted to give after purification via HPLC (method T) 20 mg (5%) of the desired title compound together with 30 mg (8%) of the regioisomer N⁴-{3-methyl-1-[(2-methyl-1,3-thiazol-4-yl)methyl]-1H-pyrazol-4-yl}quinoline-2,4-dicarboxamide.

1H NMR (400 MHz, DMSO d₆): δ (ppm)=2.33 (s, 3H), 2.63 (s, 3H), 5.32 (s, 2H), 7.23 (s, 1H), 7.74 (s, 1H), 7.79 (m, 1H), 7.88 (br.s., 1H), 7.93 (m, 1H), 8.21 (d, 1H), 8.24 (d, 1H), 8.25 (s, 1H), 8.37 (br.s., 1H), 10.28 (s, 1H).

### Example 40

### 6-bromo-N-(5-methyl-1-{[5-(methylcarbamoyl)-1,2,4-oxadiazol-3-yl]methyl}-1H-pyrazol-4-yl)-2-(trifluoromethyl)quinoline-4-carboxamide

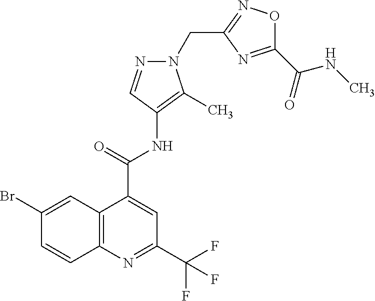 [SMILES: CNC(=O)c1nc(Cn2ncc(NC(=O)c3cc(C(F)(F)F)nc4ccc(Br)cc34)c2C)no1]

In analogy to example 1), 140 mg (0.59 mmol) of a mixture of 3-[(4-amino-5-methyl-1H-pyrazol-1-yl)methyl]-N-methyl-1,2,4-oxadiazole-5-carboxamide and 3-[(4-amino-3-methyl-1H-pyrazol-1-yl)methyl]-N-methyl-1,2,4-oxadiazole-5-carboxamide (intermediate 15C) and 166 mg (0.49 mmol) 6-bromo-2-(trifluoromethyl)quinoline-4-carboxylic acid (intermediate 1A) were reacted to give after purification via HPLC (method R) 18 mg (7%) of the desired title compound together with 14 mg (5%) of the regioisomer 6-bromo-N-(3-methyl-1-{[5-(methylcarbamoyl)-1,2,4-oxadiazol-3-yl]methyl}-1H-pyrazol-4-yl)-2-(trifluoromethyl)quinoline-4-carboxamide.

1H NMR (400 MHz, DMSO d₆): δ (ppm)=2.36 (s, 3H), 2.79 (d, 3H), 5.61 (s, 2H), 7.83 (s, 1H), 8.15 (dd, 1H), 8.23 (d, 1H), 8.28 (s, 1H), 8.47 (d, 1H), 9.32 (br.q., 1H), 10.45 (s, 1H).

### Example 41

### 6-bromo-N-{5-methyl-1-[(2-methyl-1, 3-thiazol-4-yl)methyl]-1H-pyrazol-4-yl}-2-(trifluoromethyl)quinoline-4-carboxamide

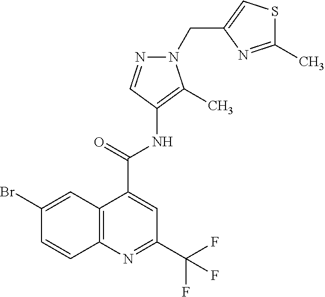 [SMILES: Cc1nc(Cn2ncc(NC(=O)c3cc(C(F)(F)F)nc4ccc(Br)cc34)c2C)cs1]

In analogy to example 1), 225 mg (1.08 mmol) of a mixture of 5-methyl-1-[(2-methyl-1,3-thiazol-4-yl)methyl]-1H-pyrazol-4-amine and 3-methyl-1-[(2-methyl-1,3-thiazol-4-yl)methyl]-1H-pyrazol-4-amine (intermediate 16C) and 288 mg (0.90 mmol) 6-bromo-2-(trifluoromethyl)quinoline-4-carboxylic acid (intermediate 1A) were reacted to give after purification via HPLC (method S) 10 mg (2%) of the desired title compound together with 40 mg (9%) of the regioisomer 6-bromo-N-{3-methyl-1-[(2-methyl-1,3-thiazol-4-yl)methyl]-1H-pyrazol-4-yl}-2-(trifluoromethyl)quinoline-4-carboxamide.

1H NMR (400 MHz, DMSO d₆): δ (ppm)=2.34 (s, 3H), 2.63 (s, 3H), 5.32 (s, 2H), 7.23 (s, 1H), 7.77 (s, 1H), 8.14 (dd, 1H), 8.22 (d, 1H), 8.26 (s, 1H), 8.47 (d, 1H), 10.37 (s, 1H).

### Example 42

### 6-bromo-N-{5-methyl-1-[(5-methyl-1,2-oxazol-3-yl)methyl]-1H-pyrazol-4-yl}-2-(trifluoromethyl)quinoline-4-carboxamide

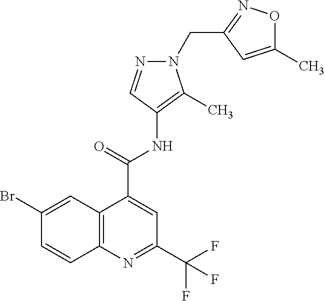 [SMILES: Cc1cc(Cn2ncc(NC(=O)c3cc(C(F)(F)F)nc4ccc(Br)cc34)c2C)no1]

In analogy to example 1), 250 mg (1.30 mmol) of a mixture of 5-methyl-1-[(5-methyl-1,2-oxazol-3-yl)methyl]-1H-pyrazol-4-amine and 3-methyl-1-[(5-methyl-1,2-oxazol-3-yl)methyl]-1H-pyrazol-4-amine (intermediate 13C) and 347 mg (1.08 mmol) 6-bromo-2-(trifluoromethyl)quinoline-4-carboxylic acid (intermediate 1A) were reacted to give after purification via HPLC (method M) 23 mg (4%) of the desired title compound together with 29 mg (5%) of the regioisomer 6-bromo-N-{3-methyl-1-[(5-methyl-1,2-oxazol-3-yl)methyl]-1H-pyrazol-4-yl}-2-(trifluoromethyl)quinoline-4-carboxamide.

1H NMR (400 MHz, DMSO d₆): δ (ppm)=2.29 (s, 3H), 2.38 (s, 3H), 5.36 (s, 2H), 6.07 (s, 1H), 7.81 (s, 1H), 8.14 (dd, 1H), 8.23 (d, 1H), 8.26 (s, 1H), 8.47 (d, 1H), 10.40 (s, 1H).

### Example 43

### N⁴-(1-{[1-(ethylsulfonyl)piperidin-4-yl]methyl}-5-methyl-1H-pyrazol-4-yl)quinoline-2,4-dicarboxamide

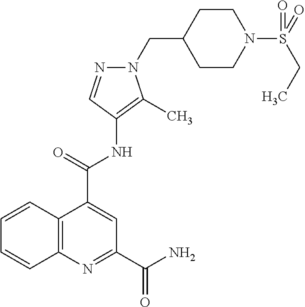 [SMILES: CCS(=O)(=O)N1CCC(Cn2ncc(NC(=O)c3cc(C(N)=O)nc4ccccc34)c2C)CC1]

In analogy to example 1), 325 mg (1.13 mmol) of a mixture of 1-{[1-(ethylsulfonyl)piperidin-4-yl]methyl}-5-methyl-1H-pyrazol-4-amine and 1-{[1-(ethylsulfonyl)piperidin-4-yl]methyl}-3-methyl-1H-pyrazol-4-amine (intermediate 17C) and 102 mg (0.47 mmol) 2-carbamoylquinoline-4-carboxylic acid (intermediate 3A) were reacted to give after purification via HPLC (method V) 26 mg (11%) of the desired title compound together with 34 mg (15%) of the regioisomer N⁴-(1-{[1-(ethylsulfonyl)piperidin-4-yl]methyl}-3-methyl-1H-pyrazol-4-yl)quinoline-2,4-dicarboxamide.

1H NMR (400 MHz, DMSO d₆): δ (ppm)=1.20 (t, 3H), 1.27 (m, 2H), 1.60 (m, 2H), 1.99 (m, 1H), 2.28 (s, 3H), 2.77 (m, 2H), 3.02 (q, 2H), 3.60 (m, 2H), 3.98 (d, 2H), 7.74 (s, 1H), 7.80 (m, 1H), 7.89 (br.s., 1H), 7.94 (m, 1H), 8.21 (d, 1H), 8.24 (d, 1H), 8.24 (s, 1H), 8.39 (br.s., 1H), 10.27 (s, 1H).

### Example 44

### 6-bromo-N-{5-methyl-1-[(1-methyl-1H-pyrazol-3-yl)methyl]-1H-pyrazol-4-yl}-2-(trifluoromethyl)quinoline-4-carboxamide

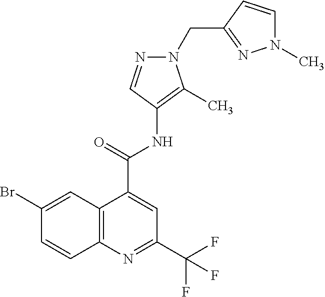 [SMILES: Cc1c(NC(=O)c2cc(C(F)(F)F)nc3ccc(Br)cc23)cnn1Cc1ccn(C)n1]

In analogy to example 1), 211 mg (1.10 mmol) of a mixture of 5-methyl-1-[(1-methyl-1H-pyrazol-3-yl)methyl]-1H-pyrazol-4-amine and 3-methyl-1-[(1-methyl-1H-pyrazol-3-yl)methyl]-1H-pyrazol-4-amine (intermediate 12C) and 294 mg (0.92 mmol) 6-bromo-2-(trifluoromethyl)quinoline-4-carboxylic acid (intermediate 1A) were reacted to give after purification via HPLC (method K) 19 mg (3%) of the desired title compound together with 41 mg (8%) of the regioisomer 6-bromo-N-{3-methyl-1-[(1-methyl-1H-pyrazol-3-yl)methyl]-1H-pyrazol-4-yl}-2-(trifluoromethyl)quinoline-4-carboxamide.

1H NMR (400 MHz, DMSO d₆): δ (ppm)=2.30 (s, 3H), 3.80 (s, 3H), 5.21 (s, 2H), 6.06 (d, 1H), 7.61 (d, 1H), 7.73 (s, 1H), 8.14 (dd, 1H), 8.22 (d, 1H), 8.26 (s, 1H), 8.46 (d, 1H), 10.36 (s, 1H).

### Example 45

### N⁴-{5-methyl-1-[(5-methyl-1,2-oxazol-3-yl)methyl]-1H-pyrazol-4-yl}quinoline-2,4-dicarboxamide

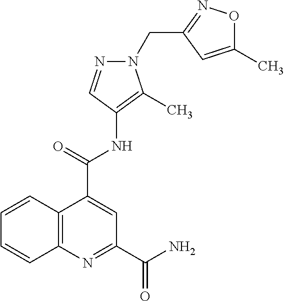 [SMILES: Cc1cc(Cn2ncc(NC(=O)c3cc(C(N)=O)nc4ccccc34)c2C)no1]

In analogy to example 1), 250 mg (1.30 mmol) of a mixture of 5-methyl-1-[(5-methyl-1,2-oxazol-3-yl)methyl]-1H-pyrazol-4-amine and 3-methyl-1-[(5-methyl-1,2-oxazol-3-yl)methyl]-1H-pyrazol-4-amine (intermediate 13C) and 234 mg (1.08 mmol) 2-carbamoylquinoline-4-carboxylic acid (intermediate 3A) were reacted to give after purification via HPLC (method N) 11 mg (2%) of the desired title compound together with 10 mg (2%) of the regioisomer N⁴-{3-methyl-1-[(5-methyl-1,2-oxazol-3-yl)methyl]-1H-pyrazol-4-yl}quinoline-2,4-dicarboxamide.

1H NMR (400 MHz, DMSO d₆): δ (ppm)=2.28 (s, 3H), 2.38 (s, 3H), 5.35 (s, 2H), 6.08 (s, 1H), 7.79 (m, 1H), 7.89 (br.s., 1H), 7.94 (m, 1H), 8.19 (d, 1H), 8.21 (d, 1H), 8.22 (s, 1H), 8.27 (s, 1H), 8.39 (br.s., 1H), 10.42 (s, 1H).

Further, the compounds of formula (I) of the present invention can be converted to any salt as described herein, by any method which is known to the person skilled in the art. Similarly, any salt of a compound of formula (I) of the present invention can be converted into the free compound, by any method which is known to the person skilled in the art.

Biological In Vitro Assays

The example testing experiments described herein serve to illustrate the present invention and the invention is not limited to the examples given.

Biological Evaluation

In order that this invention may be better understood, the following examples are set forth. These examples are for the purpose of illustration only, and are not to be construed as imiting the scope of the invention in any manner. ALL publications mentioned herein are incorporated by reference in their entirety. Demonstration of the activity of the compounds of the present invention may be accomplished through in vitro and in vivo assays that are well known in the art. For example, to demonstrate the efficacy of a pharmaceutical agent to inhibit glucose transporter GLUT1 and/or GLUT2 the following assays may be used.

Indirect Measurement of GLUT Activity by Quantification of Intracellular ATP Levels

It is well known that a combination of small-molecule inhibitors of mitochondrial electron transport chain and glucose catabolism synergistically suppress ATP production and impair cellular viability (Ulanovskaya et al., 2008, 2011; Liu, et al. 2001). We therefore used DLD1 or CHO-K1 cells in combination with an oxidative phosphorylation inhibitor to identify GLUT inhibitors. Cell lines were maintained in DMEM medium supplemented with 10% FCS and 1% Penicillin-Streptomycin solution and 2% Glutamax. The cells were treated with trypsin and seeded into 384 plates at a density of 4000 cells/well. The cells were then cultured overnight in glucose free media containing 1% FCS to reduce intracellular ATP levels. After 24 h the cells were incubated at 37° C. containing the appropriate glucose or in case of GLUT2 fructose concentration (1 mM and 30 mM respectively) with or without compounds and 1 uM Rotenone for 15 min. The CellTiter-Glo® Luminescent Cell Viability Assay from Promega was then used to measure ATP levels. Compounds able to reduce the ATP levels within 15 min of glucose application were considered to be glucose uptake inhibitors.

TABLE 1
Measured IC₅₀ values of compounds regarding
glucose induced ATP increase (GLUT1 inhibition)

| Example | IC₅₀ [nM]¹ |
|---|---|
| 1 | 9.4 |
| 2 | 124 |
| 3 | 152 |
| 4 | 12 |
| 5 | 18 |
| 6 | 91 |
| 7 | 185 |
| 8 | 12 |
| 9 | 8.9 |
| 10 | 87 |
| 11 | 19 |
| 12 | 4.0 |
| 13 | 2.2 |
| 14 | 173 |
| 15 | 755 |
| 16 | 374 |
| 17 | 10200 |
| 18 | 1250 |
| 19 | 1070 |
| 20 | 10200 |
| 21 | 3960 |
| 22 | 387 |
| 23 | 2.9 |
| 24 | 7.1 |
| 25 | 6.5 |
| 26 | 95 |
| 27 | 52 |
| 28 | 349 |
| 29 | 2150 |
| 30 | 46 |
| 31 | 201 |
| 32 | 43 |
| 33 | 940 |
| 34 | 753 |
| 35 | 1200 |
| 36 | 2220 |
| 37 | 1920 |
| 38 | 615 |
| 39 | 517 |
| 40 | 456 |
| 41 | 284 |
| 42 | 1130 |
| 43 | 396 |
| 44 | 531 |
| 45 | 222 |

¹DLD1 cells used for ATP level measurements, all IC₅₀ values were standardized to cytochalasin B IC₅₀ values;

TABLE 2
Measured IC₅₀ values of compounds regarding
fructose induced ATP increase (GLUT2 inhibition)

| | IC₅₀ |
|---|---|
| Example | [nM]¹ |
| 1 | 5990 |
| 10 | 1650 |

Biological Assay: Glucose Uptake Assay

Cells (e.g. H460 or CHO-K1) were cultured under standard conditions. 10000 cells per well were seeded in clear 96 well tissue culture isoplate plates and cultured overnight (PerkinElmer, 1450-516) under standard conditions. Culture medium was removed and cells were washed two times with 100 μL KRP buffer and then incubated for 45 minutes at 37° C. (KRP buffer: 10 mM sodium hydrogen phosphate, 130 mM sodium chloride, 5 mM potassium chloride, 1.3 mM magnesium sulfate, 1.3 mM calcium chloride (pH 7.5), 50 mM HEPES (pH 7.5), 4.7 mM potassium chloride, 1.25 mM magnesium sulfate, 1.25 mM calcium chloride) each. KRP wash buffer was removed and compound 126 (diluted in KRP buffer) was added and incubated for 30 minutes at 37° C. 200 nM radioligand (radioligand 2[1,2] 3H-Deoxy D-Glucose in KRP buffer) were added and incubated for 5 minutes at room temperature. The supernatant was removed and cells were washed with 100 μL ice-cold KRP for two times each. 25 μL of Lysis buffer (1% Triton-X, 0.5N sodium hydroxide) were added and incubated at room temperature for 5 minutes. 75 μL scintillation solution (Microscint-20, PerkinElmer) were added and the plates were shaken for 1 minute. The plates were incubated for 3 h at room temperature and the counts were determined by using a Wallace MicroBeta counter (60 seconds per well).

Biological Assay: Proliferation Assay

Cultivated tumor cells (MCF7, hormone dependent human mammary carcinoma cells, ATCC HTB22; NCI-H460, human non-small cell lung carcinoma cells, ATCC HTB-177; DU 145, hormone-independent human prostate carcinoma cells, ATCC HTB-81; HeLa-MaTu, human cervical carcinoma cells, EPO-GmbH, Berlin; HeLa-MaTu-ADR, multidrug-resistant human cervical carcinoma cells, EPO-GmbH, Berlin; HeLa human cervical tumor cells, ATCC CCL-2; B16F10 mouse melanoma cells, ATCC CRL-6475) were plated at a density of 5000 cells/well (MCF7, DU145, HeLa-MaTu-ADR), 3000 cells/well (NCI-H460, HeLa-MaTu, HeLa), or 1000 cells/well (B16F10) in a 96-well multititer plate in 200 μL of their respective growth medium supplemented 10% fetal calf serum. After 24 hours, the cells of one plate (zero-point plate) were stained with crystal violet (see below), while the medium of the other plates was replaced by fresh culture medium (200 μL), to which the test substances were added in various concentrations (0 μM, as well as in the range of 0.01-30 μM; the final concentration of the solvent dimethyl sulfoxide was 0.5%). The cells were incubated for 4 days in the presence of test substances. Cell proliferation was determined by staining the cells with crystal violet: the cells were fixed by adding 20 μL/measuring point of an 11% glutaric aldehyde solution for 15 minutes at room temperature. After three washing cycles of the fixed cells with water, the plates were dried at room temperature. The cells were stained by adding 100 μL/measuring point of a 0.1% crystal violet solution (pH 3.0). After three washing cycles of the stained cells with water, the plates were dried at room temperature. The dye was dissolved by adding 100 μL/measuring point of a 10% acetic acid solution. The extinction was determined by photometry at a wavelength of 595 nm. The change of cell number, in percent, was calculated by normalization of the measured values to the extinction values of the zero-point plate (=0%) and the extinction of the untreated (0 μm) cells (=100%). The IC₅₀ values were determined by means of a 4 parameter fit.

Determination of Metabolic Stability In Vitro

(Including Calculation of Hepatic In Vivo Blood Clearance (CL) and of Maximal Oral Bioavailability (Fₘₐₓ))

The metabolic stability of test compounds in vitro was determined by incubating them at 1 μM with a suspension liver microsomes in 100 mM phosphate buffer, pH7.4 (NaH₂PO₄×H₂O+Na₂HPO₄×2H₂O) at a protein concentration of 0.5 mg/mL and at 37° C. The reaction was activated by adding a co-factor mix containing 1.2 mg NADP, 3 IU glucose-6-phosphate dehydrogenase, 14.6 mg glucose-6-phosphate and 4.9 mg MgCl₂ in phosphate buffer, pH 7.4. Organic solvent in the incubations was imited to <0.2% dimethylsulfoxide (DMSO) and <1% methanol. During incubation, the microsomal suspensions were continuously shaken and aliquots were taken at 2, 8, 16, 30, 45 and 60 min, to which equal volumes of cold methanol were immediately added. Samples were frozen at −20° C. over night, subsequently centrifuged for 15 minutes at 3000 rpm and the supernatant was analyzed with an Agilent 1200 HPLC-system with LCMS/MS detection.

The half-life of a test compound was determined from the concentration-time plot. From the half-life the intrinsic clearances were calculated. Together with the additional parameters liver blood flow, specific liver weight and microsomal protein content the hepatic in vivo blood clearance (CL) and the maximal oral bioavailability (Fₘₐₓ) were calculated for the different species. The following parameter values were used: Liver blood flow—1.3 L/h/kg (human), 2.1 L/h/kg (dog), 4.2 L/h/kg (rat); specific liver weight—21 g/kg (human), 39 g/kg (dog), 32 g/kg (rat); microsomal protein content—40 mg/g.

With the described assay only phase-I metabolism of microsomes is reflected, e.g. typically oxidoreductive reactions by cytochrome P450 enzymes and flavin mono-oxygenases (FMO) and hydrolytic reactions by esterases (esters and amides).

### LITERATURE

Liu H, Hu Y P, Savarai N, Priebe W, Lampadis T. Hypersensitization of tumor cells to glycolytic inhibitors. Biochemistry. 2001; 40:5542-5547.
Ulanovskaya O, Janjic J, Matsumoto K, Schumacker P T, Kron S J, Kozmin S A. Synthesis enables identification of the cellular target of leucascandrolide A and neopeltolide. Nat Chem Biol. 2008; 4:418-424.
Ulanovskaya O, Jiayue Cui, Stephen J. Kron, and Sergey A. Kozmin. A pairwise chemical genetic screen identifies new inhibitors of glucose transport. Chem Biol. 2011 Feb. 25; 18(2): 222-230.

1: A compound of general formula (I): 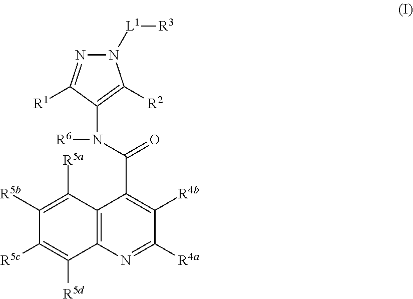
in which:
R¹ represents a hydrogen atom;
R² represents a C₁-C₃-alkyl-, halo-C₁-C₃-alkyl-, cyano-, —C(═O)O—R¹⁰ or —C(═O)N(R^{10a})R^{10b} group;
R³ represents a group selected from: phenyl-, heteroaryl-, C₅-C₆-cycloalkyl-, and 5- to 6-membered heterocycloalkyl-;
wherein said 5- to 6-membered heterocycloalkyl- group is optionally benzocondensed;
wherein said phenyl-, heteroaryl-, C₅-C₆-cycloalkyl-, and 5- to 6-membered heterocycloalkyl- group is optionally substituted, one or more times, identically or differently, with -(L²)ₚ-R⁷;
and wherein two -(L²)ₚ-R⁷ groups, if being present ortho to each other on an aryl- or heteroaryl- group optionally form a bridge selected from:
*—C₃-C₅-alkylene-*, *—O(CH₂)₂O—*, *—O(CH₂)O—*, *—O(CF₂)O—*, *—CH₂C(R^{10a})(R^{10b})O—*, *—C(═O)N(R^{10a})CH₂—*, *—N(R^{10a})C(═O)CH₂O—*, *—NHC(═O)NH—*; wherein each * represents the point of attachment to said aryl- or heteroaryl- group;
R^{4a} represents a hydrogen atom or a halogen atom or a group selected from: cyano-, hydroxy-, C₁-C₃-alkyl-, halo-C₁-C₃-alkyl-, C₁-C₃-alkoxy-, C₃-C₇-cycloalkyl-, 4- to 7-membered heterocycloalkyl-, —C(═O)N(R^{10a})R^{10b}, —N(R^{10a})R^{10b};
R^{4b} represents a hydrogen atom or a group selected from: C₁-C₃-alkoxy-, C₁-C₃-alkyl-, cyano-;
or
R^{4a} and together R^{4b} form a —C₃-C₅-alkylene- group;
R^{5a}, R^{5b}, R^{5c}, R^{5d}
independently from each other represent a hydrogen atom, a halogen atom or a group selected from:
cyano-, —NO₂, C₁-C₃-alkyl-, halo-C₁-C₃-alkyl-, C₁-C₃-alkoxy-, halo-C₁-C₃-alkoxy-, phenyl-,
heteroaryl-, —C(═O)R¹⁰, —C(═O)N(H)R¹⁰, —C(═O)N(R^{10a})R^{10b}, —C(═O)O—R¹⁰, —N(R^{10a})R^{10b}, —N(H)C(═O)R¹⁰, —N(R^{10a})C(═O)R^{10b}, —N(H)C(═O)N(R^{10a})R^{10b}, —N(R^{10a})C(═O)N(R^{10b})R^{10c}, —N(R^{10a})C(═O)C(═O)N(R^{10b})R^{10c}, —N(H)C(═O)OR¹⁰, —N(R^{10a})C(═O)OR^{10b}, —N(H)S(═O)₂R¹⁰, —N(R^{10a})S(═O)₂R^{10b}, —OR¹⁰, —O(C═O)R¹⁰, —O(C═O)N(R^{10a})R^{10b}, —O(C═O)OR¹⁰, —SR¹⁰, —S(═O)R¹⁰, —S(═O)₂R¹⁰, —S(═O)₂N(H)R¹⁰, —S(═O)₂N(R^{10a})R^{10b} or —S(═O)(=NR¹⁰a)R^{10b},
said phenyl- or heteroaryl- group being optionally substituted one or more times, identically or differently, with a group selected from:
halo-, cyano-, C₁-C₃-alkyl-, halo-C₁-C₃-alkyl-, C₁-C₃-alkoxy- group;
R⁶ represents a hydrogen atom or group selected from: C₁-C₃-alkyl-, C₁-C₃-alkoxy-(L²)-, hydroxy-C₁-C₃-alkyl-, aryl-(L²)-, heteroaryl-(L²)-;
R⁷ represents a group selected from: oxo, C₁-C₃-alkyl-, C₃-C₇-cycloalkyl-, 4- to 7-membered heterocycloalkyl-, halo-C₁-C₃-alkyl-, C₁-C₃-alkoxy-, halo-C₁-C₃-alkoxy-, —OH, —CN, halo-, —C(═O)R⁸, —C(═O)—O—R⁸, —C(═O)N(R^{8a})R^{8b}, —S(═O)₂R⁸, —S(═O)(═N)R¹¹, phenyl-, 5- to 6-membered heteroaryl-;
R⁸ represents a hydrogen atom or a C₁-C₆-alkyl-, halo-C₁-C₃-alkyl-, cyano-C₁-C₄-alkyl-, C₁-C₃-alkoxy-C₁-C₃-alkyl-, C₃-C₇-cycloalkyl-, phenyl-, 5- to 6-membered heteroaryl- or benzyl- group;
R^{8a}, R^{8b}
represent, independently from each other, a hydrogen atom, or a C₁-C₁₀-alkyl-, C₃-C₇-cycloalkyl-, (C₃-C₇-cycloalkyl)-(L³)-, C₃-C₆-alkenyl-, C₃-C₆-alkynyl-, 4- to 10-membered heterocycloalkyl-,
(4- to 10-membered heterocycloalkyl)-(L³)-, phenyl-, heteroaryl-, phenyl-(L³)-, (phenyl)-O-(L³)-, heteroaryl-(L³)-, or
(aryl)-(4- to 10-membered heterocycloalkyl)- group;
said C₁-C₁₀-alkyl-, C₃-C₇-cycloalkyl-, (C₃-C₇-cycloalkyl)-(L³)-, C₃-C₆-alkenyl-, C₃-C₆-alkynyl-, 4- to 10-membered heterocycloalkyl-, (4- to 10-membered heterocycloalkyl)-(L³)-, phenyl-, heteroaryl-, phenyl-(L³)-, (phenyl)-O-(L³)-, heteroaryl-(L³)-, and (aryl)-(4- to 10-membered heterocycloalkyl)- group being optionally substituted one or more times, identically or differently, with R⁹;
or
R^{8a} and R^{8b}, together with the nitrogen atom they are attached to,
represent a 4- to 10-membered heterocycloalkyl-group, said 4- to 10-membered heterocycloalkyl-group being optionally substituted one or more times, identically or differently, with R⁹;
R⁹ represents a halogen atom, or a oxo, C₁-C₃-alkyl-, halo-C₁-C₃-alkyl-, hydroxy-C₁-C₃-alkyl-, —CN, —C(═O)R¹⁰, —C(═O)N(H)R¹⁰, —C(═O)N(R^{10a})R^{10b}, —C(═O)O—R¹⁰, —N(R^{10a})R^{10b}, —NO₂, —N(H)C(═O)R¹⁰, —N(R^{10a})C(═O)R^{10b}, —N(H)C(═O)N(R^{10a})R^{10b}, —N(R^{10a})C(═O)N(R^{10b})R^{10c}, —N(H)C(═O)OR¹⁰, —N(R^{10a})C(═O)OR^{10b}, —N(H)S(═O)₂R¹⁰, —N(R^{10a})S(═O)₂R^{10b}, —OR¹⁰, —O(C═O)R¹⁰, —O(C═O)N(R^{10a})R^{10b}, —O(C═O)OR¹⁰, —SR¹⁰, —S(═O)R¹⁰, —S(═O)₂R¹⁰, —S(═O)₂N(H)R¹⁰, —S(═O)₂N(R^{10a})R^{10b}, —S(═O)(═NR^{10a})R^{10b} or a tetrazolyl-group;
or
two R⁹ groups present ortho to each other on a phenyl- or heteroaryl- ring form a bridge selected from: *—C₃-C₅-alkylene-*, *—O(CH₂)₂O—*, *—O(CH₂)O—*, *—O(CF₂)O—*, *—CH₂C(R^{10a})(R^{10b})O—*, *—C(═O)N(R^{10a})CH₂—*, *—N(R^{10a})C(═O)CH₂O—*, *—NHC(═O)NH—*; wherein each * represents the point of attachment to said phenyl- or heteroaryl- ring;
R¹⁰, R^{10a}, R^{10b}, R^{10c}
represent, independently from each other, a hydrogen atom or a group selected from: C₁-C₃-alkyl-, halo-C₁-C₃-alkyl-, hydroxy-C₁-C₃-alkyl-, C₁-C₃-alkoxy-C₁-C₃-alkyl-, C₃-C₇-cycloalkyl-;
R¹¹ represents a hydrogen atom or a cyano-, C₁-C₃-alkyl-, —C(═O)R¹⁰, —C(═O)N(H)R¹⁰, —C(═O)N(R^{10a})R^{10b} or —C(═O)O—R¹⁰ group;
L¹ represents a group selected from: —C₁-C₄-alkylene-, —CH₂—CH═CH—, —C(phenyl)(H)—, —CH₂—CH₂—O—;
L² represents a group selected from: —CH₂—, —CH₂—CH₂—, —CH₂—CH₂—CH₂—;
L³ represents a —C₁-C₆-alkylene- group;
p is an integer of 0 or 1;
or a tautomer, a stereoisomer, an N-oxide, a hydrate, a solvate, or a salt thereof, or a mixture of same.

2: The compound according to claim 1, wherein
R² represents a C₁-C₃-alkyl-, —C(═O)O—R¹⁰ or —C(═O)N(R^{10a})R^{10b} group.

3: The compound according to claim 1, wherein
R³ represents a group selected from: phenyl-, heteroaryl-, C₅-C₆-cycloalkyl-, and 5- to 6-membered heterocycloalkyl-;
wherein said phenyl-, heteroaryl-, C₅-C₆-cycloalkyl-, and 5- to 6-membered heterocycloalkyl- group is optionally substituted, one or two times, identically or differently, with —R⁷.

4: The compound according to claim 1, wherein
R^{4a} represents a group selected from: C₁-C₃-alkyl-, halo-C₁-C₃-alkyl-, C₁-C₃-alkoxy-, C₃-C₇-cycloalkyl-, —C(═O)N(R^{10a})R^{10b}.

5: The compound according to claim 1, wherein
R^{4b} represents a hydrogen atom.

6: The compound according to claim 1, wherein
R^{5a}, R^{5b}, R^{5c}, R^{5d}
independently from each other represent a hydrogen atom, a halogen atom or a C1-C₃-alkyl-group.

7: The compound according to claim 1, wherein
R⁶ represents a hydrogen atom.

8: The compound according to claim 1, wherein
L¹ represents —CH₂—.

9: The compound according to claim 1, wherein
R⁷ represents a group selected from: C₁-C₃-alkyl-, C₁-C₃-alkoxy-, —CN, halo-, —C(═O)N(R^{8a})R^{8b}, —S(═O)₂R⁸.

10: The compound according to claim 1, wherein
R³ represents a phenyl- group; wherein said phenyl- group is substituted, one or two times, with fluoro; or
R³ represents a phenyl- group; wherein said phenyl- group is substituted, one time, with cyano; or
R³ represents a phenyl- group; wherein said phenyl- group is substituted, one time, with methoxy; or
R³ represents a pyrazolyl- group; wherein said group is substituted with a methyl group; or
R³ represents an isoxazolyl- group; wherein said group is substituted with a methyl group; or
R³ represents a thiazolyl- group; wherein said group is substituted with a methyl group; or
R³ represents an oxadiazolyl- group; wherein said group is substituted with a group selected from ethyl-, —C(═O)N(H)CH₃; or
R³ represents a pyridyl- group; or
R³ represents a cyclohexyl- group; or
R³ represents a piperidinyl- group; wherein said group is substituted with a —S(═O)₂—CH₂—CH₃ group.

11: The compound according to claim 1, which is selected from the group consisting of:
N-[1-(4-fluorobenzyl)-5-methyl-1H-pyrazol-4-yl]-2,6-dimethylquinoline-4-carboxamide;
6,7-difluoro-N-[1-(4-fluorobenzyl)-5-methyl-1H-pyrazol-4-yl]-2-(trifluoromethyl)quinoline-4-carboxamide;
N-[1-(4-fluorobenzyl)-5-methyl-1H-pyrazol-4-yl]-2-methoxyquinoline-4-carboxamide;
6-bromo-N-[1-(4-fluorobenzyl)-5-methyl-1H-pyrazol-4-yl]-2-(trifluoromethyl)quinoline-4-carboxamide;
N⁴-[1-(4-fluorobenzyl)-5-methyl-1H-pyrazol-4-yl]quinoline-2,4-dicarboxamide;
2-cyclopropyl-6-fluoro-N-[1-(4-fluorobenzyl)-5-methyl-1H-pyrazol-4-yl]quinoline-4-carboxamide;
6,8-dichloro-N-[1-(4-fluorobenzyl)-5-methyl-1H-pyrazol-4-yl]-2-(trifluoromethyl)quinoline-4-carboxamide;
6-bromo-N-[1-(4-cyanobenzyl)-5-methyl-1H-pyrazol-4-yl]-2-(trifluoromethyl)quinoline-4-carboxamide;
N⁴-[1-(4-cyanobenzyl)-5-methyl-1H-pyrazol-4-yl]quinoline-2,4-dicarboxamide;
N-[1-(4-cyanobenzyl)-5-methyl-1H-pyrazol-4-yl]-2-methoxyquinoline-4-carboxamide;
2-methoxy-N-[1-(4-methoxybenzyl)-5-methyl-1H-pyrazol-4-yl]quinoline-4-carboxamide;
6-bromo-N-[1-(4-methoxybenzyl)-5-methyl-1H-pyrazol-4-yl]-2-(trifluoromethyl)quinoline-4-carboxamide;
N⁴-[1-(4-methoxybenzyl)-5-methyl-1H-pyrazol-4-yl]quinoline-2,4-dicarboxamide;
6-bromo-N-[1-(2-cyanobenzyl)-5-methyl-1H-pyrazol-4-yl]-2-(trifluoromethyl)quinoline-4-carboxamide;
N⁴-[1-(2-cyanobenzyl)-5-methyl-1H-pyrazol-4-yl]quinoline-2,4-dicarboxamide;
methyl 4-({[6-bromo-2-(trifluoromethyl)quinolin-4-yl]carbonyl}amino)-1-(4-cyanobenzyl)-1H-pyrazole-5-carboxylate;
4-({[6-bromo-2-(trifluoromethyl)quinolin-4-yl]carbonyl}amino)-1-(4-fluorobenzyl)-1H-pyrazole-5-carboxylic acid;
6-bromo-N-[5-carbamoyl-1-(4-fluorobenzyl)-1H-pyrazol-4-yl]-2-(trifluoromethyl)quinoline-4-carboxamide;
methyl 4-({[6-bromo-2-(trifluoromethyl)quinolin-4-yl]carbonyl}amino)-1-(4-cyanobenzyl)-1H-pyrazole-5-carboxylate;
4-({[6-bromo-2-(trifluoromethyl)quinolin-4-yl]carbonyl}amino)-1-(4-cyanobenzyl)-1H-pyrazole-5-carboxylic acid;
6-bromo-N-[1-(4-cyanobenzyl)-5-(methylcarbamoyl)-1H-pyrazol-4-yl]-2-(trifluoromethyl)quinoline-4-carboxamide;
6-bromo-N-[5-carbamoyl-1-(4-cyanobenzyl)-1H-pyrazol-4-yl]-2-(trifluoromethyl)quinoline-4-carboxamide;
6-bromo-N-[1-(4-cyanobenzyl)-5-methyl-1H-pyrazol-4-yl]-2-cyclopropylquinoline-4-carboxamide;
N⁴-[1-(4-cyanobenzyl)-5-methyl-1H-pyrazol-4-yl]-7-fluoroquinoline-2,4-dicarboxamide;
6-chloro-N⁴-[1-(4-cyanobenzyl)-5-methyl-1H-pyrazol-4-yl]-7-fluoroquinoline-2,4-dicarboxamide;
N⁴-[1-(3,4-difluorobenzyl)-5-methyl-1H-pyrazol-4-yl]quinoline-2,4-dicarboxamide;
N⁴-[1-(3-fluorobenzyl)-5-methyl-1H-pyrazol-4-yl]quinoline-2,4-dicarboxamide;
N⁴-[1-(cyclohexylmethyl)-5-methyl-1H-pyrazol-4-yl]quinoline-2,4-dicarboxamide;
N⁴-[5-methyl-1-(pyridin-4-ylmethyl)-1H-pyrazol-4-yl]quinoline-2,4-dicarboxamide;
N⁴-[1-(4-cyanobenzyl)-5-ethyl-1H-pyrazol-4-yl]-7-fluoroquinoline-2,4-dicarboxamide;
N⁴-[1-(4-cyanobenzyl)-5-ethyl-1H-pyrazol-4-yl]quinoline-2,4-dicarboxamide;
6-chloro-N⁴-[1-(4-cyanobenzyl)-5-ethyl-1H-pyrazol-4-yl]-7-fluoroquinoline-2,4-dicarboxamide;
N⁴-[1-(4-cyanobenzyl)-5-isopropyl-1H-pyrazol-4-yl]-7-fluoroquinoline-2,4-dicarboxamide;
N⁴-{5-methyl-1-[(1-methyl-1H-pyrazol-3-yl)methyl]-1H-pyrazol-4-yl}quinoline-2,4-dicarboxamide;
6-bromo-N-{1-[(3-ethyl-1,2,4-oxadiazol-5-yl)methyl]-5-methyl-1H-pyrazol-4-yl}-2-(trifluoromethyl)quinoline-4-carboxamide;
N⁴-(5-methyl-1-{[5-(methylcarbamoyl)-1,2,4-oxadiazol-3-yl]methyl}-1H-pyrazol-4-yl)quinoline-2,4-dicarboxamide;
N⁴-{1-[(3-ethyl-1,2,4-oxadiazol-5-yl)methyl]-5-methyl-1H-pyrazol-4-yl}quinoline-2,4-dicarboxamide;
6-bromo-N-(1-{[1-(ethylsulfonyl)piperidin-4-yl]methyl}-5-methyl-1H-pyrazol-4-yl)-2-(trifluoromethyl)quinoline-4-carboxamide;
N⁴-{5-methyl-1-[(2-methyl-1,3-thiazol-4-yl)methyl]-1H-pyrazol-4-yl}quinoline-2,4-dicarboxamide;
6-bromo-N-(5-methyl-1-{[5-(methylcarbamoyl)-1,2,4-oxadiazol-3-yl]methyl}-1H-pyrazol-4-yl)-2-(trifluoromethyl)quinoline-4-carboxamide;
6-bromo-N-{5-methyl-1-[(2-methyl-1,3-thiazol-4-yl)methyl]-1H-pyrazol-4-yl}-2-(trifluoromethyl)quinoline-4-carboxamide;
6-bromo-N-{5-methyl-1-[(5-methyl-1,2-oxazol-3-yl)methyl]-1H-pyrazol-4-yl}-2-(trifluoromethyl)quinoline-4-carboxamide;
N⁴-(1-{[1-(ethylsulfonyl)piperidin-4-yl]methyl}-5-methyl-1H-pyrazol-4-yl)quinoline-2,4-dicarboxamide;
6-bromo-N-{5-methyl-1-[(1-methyl-1H-pyrazol-3-yl)methyl]-1H-pyrazol-4-yl}-2-(trifluoromethyl)quinoline-4-carboxamide; and
N⁴-{5-methyl-1-[(5-methyl-1,2-oxazol-3-yl)methyl]-1H-pyrazol-4-yl}quinoline-2,4-dicarboxamide;
or a tautomer, an N-oxide, a hydrate, a solvate, or a salt thereof, or a mixture of same.

12: A method of preparing a compound of general formula (I) according to claim 1, in which method an intermediate of general formula (II) 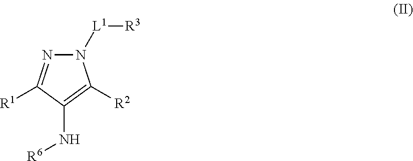
in which R¹, R², R³, R⁶ and L¹ are as defined in claim 1;
is allowed to react with a compound of general formula (III) 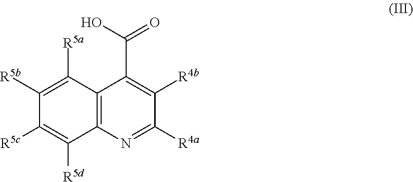
in which R^{4a}, R^{4b}, R^{5a}, R^{5b}, R^{5c}, and R^{5d} are as defined in claim 1;
thus providing a compound of general formula (I) 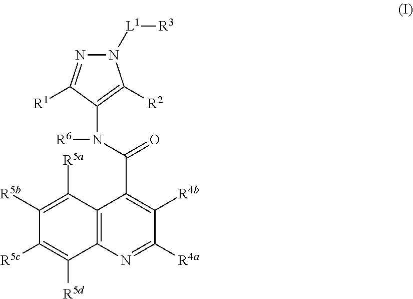
in which R¹, R², R³, R^{4a}, R^{4b}, R^{5a}, R^{5b}, R^{5b}, R^{5d}, R⁶, and L¹ are as defined in claim 1.

13: A compound of general formula (II) 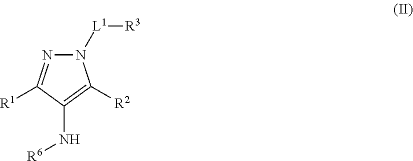
in which R¹, R², R³, R⁶ and L¹ are as defined in claim 1.

14: (canceled)

15: A method for the treatment or prophylaxis of a disease, said method comprising administering to a patient in need thereof a compound according to claim 1, or a tautomer, an N-oxide, a hydrate, a solvate, or a salt thereof, particularly a pharmaceutically acceptable salt thereof, or a mixture of same.

16: A pharmaceutical composition comprising a compound of formula (I) as defined in claim 1, or a tautomer, an N-oxide, a hydrate, a solvate, or a salt thereof, particularly a pharmaceutically acceptable salt thereof, or a mixture of same, and a pharmaceutically acceptable diluent or carrier.

17: A pharmaceutical combination comprising:
one or more compounds of formula (I) according to claim 1, or a tautomer, an N-oxide, a hydrate, a solvate, or a salt thereof, particularly a pharmaceutically acceptable salt thereof, or a mixture of same;
and
one or more agents selected from: a taxane, such as Docetaxel, Paclitaxel, or Taxol; an epothilone, such as Ixabepilone, Patupilone, or Sagopilone; Mitoxantrone; Predinisolone; Dexamethasone; Estramustin; Vinblastin; Vincristin; Doxorubicin; Adriamycin; Idarubicin; Daunorubicin; Bleomycin; Etoposide; Cyclophosphamide; Ifosfamide; Procarbazine; Melphalan; 5-Fluorouracil; Capecitabine; Fludarabine; Cytarabine; Ara-C; 2-Chloro-2′-deoxyadenosine; Thioguanine; an anti-androgen, such as Flutamide, Cyproterone acetate, or Bicalutamide; Bortezomib; a platinum derivative, such as Cisplatin, or Carboplatin; Chlorambucil; Methotrexate; and Rituximab.

18-19. (canceled)

20: The method according to claim 15, wherein the disease is a disease of uncontrolled cell growth, proliferation and/or survival, an inappropriate cellular immune response, or an inappropriate cellular inflammatory response, particularly in which the uncontrolled cell growth, proliferation and/or survival, inappropriate cellular immune response, or inappropriate cellular inflammatory response is mediated by GLUT1, more particularly in which the disease of uncontrolled cell growth, proliferation and/or survival, inappropriate cellular immune response, or inappropriate cellular inflammatory response is a haemotological tumour, a solid tumour and/or metastases thereof, e.g. leukaemias and myelodysplastic syndrome, malignant lymphomas, head and neck tumours including brain tumours and brain metastases, tumours of the thorax including non-small cell and small cell lung tumours, gastrointestinal tumours, endocrine tumours, mammary and other gynaecological tumours, urological tumours including renal, bladder and prostate tumours, skin tumours, and sarcomas, and/or metastases thereof.
